(19) United States
(12) Patent Application Publication
(10) Pub. No.: US 2026/0242873 A1
(43) Pub. Date: Aug. 20, 2026

(54) NOVEL SIGNATURES FOR LUNG CANCER DETECTION

(71) Applicant: Hummingbird Diagnostics GmbH, Heidelberg (DE)

(72) Inventors: Rastislav Horos, Heidelberg (DE); Tobias Sikosek, Heidelberg (DE); Bruno Steinkraus, Hanstedt (DE)

(21) Appl. No.: 18/876,767

(22) PCT Filed: May 26, 2023
(86) PCT No.: PCT/EP2023/064236

§ 371 Date: Dec. 19, 2024

(30) Foreign Application Priority Data
Jun. 28, 2022 (EP) 22181710.9

(51) Int. Cl. C12Q 1/6886

(52) U.S. Cl. CPC C12Q 1/6886; C12Q 2600/158

(57) ABSTRACT

The present invention relates to a method for diagnosing lung cancer in a patient. In addition, the present invention relates to a method for monitoring lung cancer in a patient. Moreover, the present invention relates to a kit for carrying out these methods.

The present invention relates to a method for diagnosing lung cancer in a patient. In addition, the present invention relates to a method for monitoring lung cancer in a patient. Moreover, the present invention relates to a kit for carrying out these methods.

### BACKGROUND OF THE INVENTION

Cancer is the uncontrolled growth of abnormal cells anywhere in a body. The abnormal cells are termed cancer cells, malignant cells, or tumor cells. Cancer cells can proliferate uncontrollably and form a mass of cancer cells.

The most common types of cancer in males are lung cancer, prostate cancer, colorectal cancer, and stomach cancer. In females, the most common types are breast cancer, colorectal cancer, lung cancer, and cervical cancer.

Lung cancer or bronchogenic carcinoma refers to tumors originating in the lung parenchyma or within the bronchi. It is one of the leading causes of cancer-related deaths in Europe and in the United States. Since 1987, lung cancer has been responsible for more deaths in women than breast cancer. It is estimated that there are 225,000 new cases of lung cancer in the United States annually, and approximately 160,000 die because of lung cancer. It is interesting to note that lung cancer was a relatively rare disease at the beginning of the 20th century. Its dramatic rise in later decades is attributable primarily to the increase in smoking among both males and females.

Lung cancer is usually diagnosed via diverse imaging techniques such as radiographic examination and/or computer tomography (CT), but more cost effective, easier to use and faster techniques are clearly needed.

Molecular studies have identified several diagnostic markers of lung cancer. However, their diagnostic value for lung cancer has been low so far. Thus, more sensitive biomarkers are needed. In addition, there are currently no standard blood or laboratory tests that have been proven to help in diagnosing lung cancer.

The present inventors identified new RNA molecules which are significantly deregulated in lung cancer patients compared to healthy controls. It has turned out that signatures of these new RNA molecules are of high diagnostic relevance for lung cancer determination. These new RNA molecules can, thus, be used as biomarkers for lung cancer diagnosis and as biomarkers for lung cancer monitoring.

### SUMMARY OF THE INVENTION

In a first aspect, the present invention relates to a method of diagnosing lung cancer in a patient comprising the step of:
(i) determining the level of at least one RNA molecule in a blood sample from a patient, wherein the at least one RNA molecule comprises a nucleotide sequence according to SEQ ID NO: 1, is a fragment thereof, or has a sequence having at least 80% sequence identity thereto,
(ii) determining the levels of at least two RNA molecules in a blood sample from a patient, wherein the at least two RNA molecules comprise a first RNA molecule and a second RNA molecule, wherein the first RNA molecule comprises a nucleotide sequence according to SEQ ID NO: 1, is a fragment thereof, or has a sequence having at least 80% sequence identity thereto and the second RNA molecule comprises a nucleotide sequence according to SEQ ID NO: 2, is a fragment thereof, or has a sequence having at least 80% sequence identity thereto, or
(iii) determining the levels of at least two RNA molecules in a blood sample from a patient, wherein the at least two RNA molecules comprise a first RNA molecule and a second RNA molecule,
   wherein the first RNA molecule comprises a nucleotide sequence according to SEQ ID NO: 2, is a fragment thereof, or has a sequence having at least 80% sequence identity thereto and the second RNA molecule comprises a nucleotide sequence according to SEQ ID NO: 3, is a fragment thereof, or has a sequence having at least 80% sequence identity thereto.

In a second aspect, the present invention relates to a method of monitoring a patient suspected of having lung cancer or a patient suffering from lung cancer comprising the step of:
(i) determining the level of at least one RNA molecule in a blood sample from a patient, wherein the at least one RNA molecule comprises a nucleotide sequence according to SEQ ID NO: 1, is a fragment thereof, or has a sequence having at least 80% sequence identity thereto,
(ii) determining the levels of at least two RNA molecules in a blood sample from a patient, wherein the at least two RNA molecules comprise a first RNA molecule and a second RNA molecule,
   wherein the first RNA molecule comprises a nucleotide sequence according to SEQ ID NO: 1, is a fragment thereof, or has a sequence having at least 80% sequence identity thereto and the second RNA molecule comprises a nucleotide sequence according to SEQ ID NO: 2, is a fragment thereof, or has a sequence having at least 80% sequence identity thereto, or
(iii) determining the levels of at least two RNA molecules in a blood sample from a patient, wherein the at least two RNA molecules comprise a first RNA molecule and a second RNA molecule,
   wherein the first RNA molecule comprises a nucleotide sequence according to SEQ ID NO: 2, is a fragment thereof, or has a sequence having at least 80% sequence identity thereto and the second RNA molecule comprises a nucleotide sequence according to SEQ ID NO: 3, is a fragment thereof, or has a sequence having at least 80% sequence identity thereto.

In a third aspect, the present invention relates to the use of
(i) at least one RNA molecule for diagnosing lung cancer in a patient, or for monitoring a patient suspected of having lung cancer or a patient suffering from lung cancer,
   wherein the at least one RNA molecule comprises a nucleotide sequence according to SEQ ID NO: 1, is a fragment thereof, or has a sequence having at least 80% sequence identity thereto,
(ii) at least two RNA molecules for diagnosing lung cancer in a patient, or for monitoring a patient suspected of having lung cancer or a patient suffering from lung cancer, wherein the at least two RNA molecules comprise a first RNA molecule and a second RNA molecule,
   wherein the first RNA molecule comprises a nucleotide sequence according to SEQ ID NO: 1, is a fragment thereof, or has a sequence having at least 80% sequence identity thereto and the second RNA molecule comprises a nucleotide sequence according to SEQ ID NO: 2, is a fragment thereof, or has a sequence having at least 80% sequence identity thereto, or
(iii) at least two RNA molecules for diagnosing lung cancer in a patient, or for monitoring a patient suspected of having lung cancer or a patient suffering from lung cancer, wherein the at least two RNA molecules comprise a first RNA molecule and a second RNA molecule,
   wherein the first RNA molecule comprises a nucleotide sequence according to SEQ ID NO: 2, is a fragment thereof, or has a sequence having at least 80% sequence identity thereto and the second RNA molecule comprises a nucleotide sequence according to SEQ ID NO: 3, is a fragment thereof, or has a sequence having at least 80% sequence identity thereto.

In a fourth aspect, the present invention relates to the use of a kit for diagnosing lung cancer in a patient, or for monitoring a patient suspected of having lung cancer or a patient suffering from lung cancer, said kit comprises
(i) means for determining the level of at least one RNA molecule in a blood sample from a patient,
   wherein the at least one RNA molecule comprises a nucleotide sequence according to SEQ ID NO: 1, is a fragment thereof, or has a sequence having at least 80% sequence identity thereto,
(ii) means for determining the levels of at least two RNA molecules in a blood sample from a patient, wherein the at least two RNA molecules comprise a first RNA molecule and a second RNA molecule,
   wherein the first RNA molecule comprises a nucleotide sequence according to SEQ ID NO: 1, is a fragment thereof, or has a sequence having at least 80% sequence identity thereto and the second RNA molecule comprises a nucleotide sequence according to SEQ ID NO: 2, is a fragment thereof, or has a sequence having at least 80% sequence identity thereto, or
(iii) means for determining the levels of at least two RNA molecules in a blood sample from a patient, wherein the at least two RNA molecules comprise a first RNA molecule and a second RNA molecule,
   wherein the first RNA molecule comprises a nucleotide sequence according to SEQ ID NO: 2, is a fragment thereof, or has a sequence having at least 80% sequence identity thereto and the second RNA molecule comprises a nucleotide sequence according to SEQ ID NO: 3, is a fragment thereof, or has a sequence having at least 80% sequence identity thereto.

This summary of the invention does not necessarily describe all features of the present invention. Other embodiments will become apparent from a review of the ensuing detailed description.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention. In the event of a conflict between the definitions or teachings of such incorporated references and definitions or teachings recited in the present specification, the text of the present specification takes precedence.

The term “comprise” or variations such as “comprises” or “comprising” according to the present invention means the inclusion of a stated integer or group of integers but not the exclusion of any other integer or group of integers. The term “consisting essentially of” according to the present invention means the inclusion of a stated integer or group of integers, while excluding modifications or other integers which would materially affect or alter the stated integer. The term “consisting of” or variations such as “consists of” according to the present invention means the inclusion of a stated integer or group of integers and the exclusion of any other integer or group of integers.

The terms “a” and “an” and “the” and similar reference used in the context of describing the invention (especially in the context of the claims) are to be construed to cover both the singular and the plural, unless otherwise indicated herein or clearly contradicted by context.

As used herein, the term “about” indicates a certain variation from the quantitative value it precedes. In particular, the term “about” allows a ±5% variation from the quantitative value it precedes, unless otherwise indicated or inferred. The use of the term “about” also includes the specific quantitative value itself, unless explicitly stated otherwise. For example, the expression “about 80° C.” allows a variation of +4° C., thus referring to range from 76° C. to 84° C.

The term “sequence identity”, as used herein, refers to a measurement which allows to indicate the similarity of nucleotide and amino acid sequences. The percentage of sequence identity can be determined via sequence alignments. Such alignments can be carried out with several art-known algorithms, preferably with the mathematical algorithm of Karlin and Altschul (Karlin & Altschul (1993) Proc. Natl. Acad. Sci. USA 90:5873-5877), with hmmalign (HMMER package) or with the CLUSTAL algorithm (Thompson, J. D., Higgins, D. G. & Gibson, T. J. (1994) Nucleic Acids Res. 22, 4673-80) or the CLUSTALW2 algorithm (Larkin M A, Blackshields G, Brown N P, Chenna R, McGettigan P A, McWilliam H, Valentin F, Wallace I M, Wilm A, Lopez R, Thompson J D, Gibson T J, Higgins D G. (2007). Clustal W and Clustal X version 2.0. Bioinformatics, 23, 2947-2948).

The grade of sequence identity (sequence matching) may be calculated using e.g. BLAST, BLAT or BlastZ (or BlastX). A similar algorithm is incorporated into the BLASTN and BLASTP programs of Altschul et al. (1990) J. Mol. Biol. 215:403-410. BLAST protein searches are performed with the BLASTP program available e.g. on the web site:
http://blast.ncbi.nlm.nih.gov/Blast.cgi?PROGRAM=blastp&BLAST PROGRAMS=blastp&PA GE TYPE=BlastSearch&SHOW_DEFAULTS=on&LINK LOC=blasthome

Preferred algorithm parameters used are the default parameters as they are set on the indicated web site:
Expect threshold=10, word size=3, max matches in a query range=0, matrix=BLOSUM62, gap costs=Existence: 11 Extension: 1, compositional adjustments=conditional compositional score matrix adjustment together with the database of non-redundant protein sequences (nr).

To obtain gapped alignments for comparative purposes, Gapped BLAST is utilized as described in Altschul et al. (1997) Nucleic Acids Res. 25:3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields.

When percentages of sequence identity are referred to in the present application, these percentages can be calculated in relation to the full length of the indicated reference sequence, if not specifically indicated otherwise. For example, the statement “a nucleotide sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to SEQ ID NO: XYZ means that the sequence identity percentage is calculated in relation to the total length of SEQ ID NO: XYZ. Herein specifically described are variants of nucleotide sequences according to SEQ ID NO: 1 to SEQ ID NO: 71. Said variants have at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequences according to SEQ ID NO: 1 to SEQ ID NO: 71. When percentages of sequence identity between two sequences are calculated, these percentages can also be calculated in relation to the full length of the longer of the two sequences, if not specifically indicated otherwise. For example, the statement “a nucleotide sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to SEQ ID NO: XYZ” means that the sequence identity percentage is calculated in relation to the longer sequence of the two sequences.

The term “RNA molecule”, as used herein, refers to a polymeric molecule essential in various biological roles in coding, decoding, regulation, and expression of genes. Ribonucleic acids (RNA) and deoxyribonucleic acids (DNA) are nucleic acids. Along with lipids, proteins, and carbohydrates, nucleic acids constitute one of the four major macromolecules essential for all known forms of life. Like DNA, RNA is assembled as a chain of nucleotides, but unlike DNA, RNA is found in nature as a single strand folded onto itself, rather than a paired double strand. Cellular organisms use messenger (mRNA) to convey genetic information (using the nitrogenous bases of guanine, uracil, adenine, and cytosine, denoted by the letters G, U, A, and C) that directs synthesis of specific proteins. Some RNA molecules play an active role within cells by catalysing biological reactions, controlling gene expression or sensing and communicating responses to cellular signals. One of these active processes is protein synthesis, a universal function in which RNA molecules direct the synthesis of proteins on ribosomes. This process uses transfer RNA ((RNA) molecules to deliver amino acids to the ribosome, where ribosomal RNA (rRNA) then links amino acids together to form coded proteins.

The term “small RNA molecule”, as described herein, refers to a polymeric RNA molecule that is usually less than 200 ribonucleotides, preferably less than 50 ribonucleotides, in length. Specifically, a small RNA molecule has a length of between 10 and <200 ribonucleotides. More specifically, a small RNA molecule has a length of between 10 and <50 ribonucleotides. The RNA molecules having a nucleotide sequence according to SEQ ID NO: 1 to SEQ ID NO: 79 are small RNA molecules.

The term “non-coding small RNA molecule”, as used herein, refers an RNA molecule that is not translated into a protein. In particular, a non-coding small RNA molecule can be defined by length. A non-coding small RNA molecule is usually less than 200 ribonucleotides, preferably less than 50 ribonucleotides, in length. Specifically, a small non-coding RNA molecule has a length of between 10 and <200 ribonucleotides. More specifically, a small RNA molecule has a length of between 10 and <50 ribonucleotides. Abundant and functionally important types of non-coding RNAs include transfer RNAs (tRNAs), ribosomal RNAs (rRNAs), microRNAs (miRNAs), small interfering RNAs (siRNAs), or piwi-interacting RNAs (piRNAs). The RNA molecules having a nucleotide sequence according to SEQ ID NO: 1 to SEQ ID NO: 79 are non-coding small RNA molecules. The RNA molecule comprising a nucleotide sequence according to SEQ ID NO: 2 is, for example, a 28S ribosomal RNA (rRNA) fragment which is significantly upregulated in lung cancer patients compared to healthy controls. Non-coding small RNA molecules include miRNAs and isomiRs.

The term “miRNA” (the designation “microRNA” is also possible), as used herein, refers to a single-stranded RNA molecule. The miRNA may be a molecule of between 10 to 50 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, not including optionally labels and/or elongated sequences (e.g. biotin stretches).

The miRNAs regulate gene expression and are encoded by genes from whose DNA they are transcribed but miRNAs are not translated into protein (i.e. miRNAs are non-coding RNAs). The genes encoding miRNAs are longer than the processed mature miRNA molecules. The miRNA is initially transcribed as a longer precursor molecule (>1000 nucleotides long) called a primary miRNA transcript (pri-miRNA). Pri-miRNAs have hairpin structures that are processed by the Drosha enzyme (as part of the microprocessor complex). After Drosha processing, the pri-miRNAs are only 60-100 nucleotides long, and are called precursor miRNAs (pre-miRNAs). At this point, the pre-miRNA is exported to the cytoplasm, where it encounters the Dicer enzyme. Dicer cuts the miRNA in two, resulting in duplexed miRNA strands. Traditionally, only one of these miRNA arms was considered important in gene regulation: the arm that is destined to be loaded into the RNA-induced silencing complex (RISC), and occurs at a higher concentration in the cell. This is often called the “guide” strand and is designated as miR. The other arm is called the “minor miRNA” or “passenger miRNA”, and is often designated as miR*. It was thought that passenger miRNAs were completely degraded, but deep sequencing studies have found that some minor miRNAs persist and in fact have a functional role in gene regulation. Due to these developments, the naming convention has shifted. Instead of the miR/miR* name scheme, a miR-5p/miR-3p nomenclature has been adopted. By the new system, the 5′ arm of the miRNA is always designated miR-5p and the 3′ arm is miR-3p. The present nomenclature is as follows: The prefix “miR” is followed by a dash and a number, the latter often indicating order of naming. For example, hsa-miR-16 was named and likely discovered prior to hsa-miR-342. A capitalized “miR-” refers to the mature forms of the miRNA (e.g. hsa-miR-16-5p and hsa-miR-16-3p), while the uncapitalized “mir-” refers to the pre-miRNA and the pri-miRNA (e.g. hsa-mir-16), and “MIR” refers to the gene that encodes them. However, as this is a recent change, literature will often refer to the original miR/miR* names. After processing, the duplexed miRNA strands are loaded onto an Argonaute (AGO) protein to form a precursor to the RISC. The complex causes the duplex to unwind, and the passenger RNA strand is discarded, leaving behind a mature RISC carrying the mature, single stranded miRNA. The miRNA remains part of the RISC as it silences the expression of its target genes. While this is the canonical pathway for miRNA biogenesis, a variety of others have been discovered. These include Drosha-independent pathways (such as the mirtron pathway, snoRNA-derived pathway, and shRNA-derived pathway) and Dicer-independent pathways (such as one that relies on AGO for cleavage, and another which is dependent on tRNaseZ).

The term “miRBase”, as used herein, refers to a well-established repository of validated miRNAs. The miRBase (www.mirbase.org) is a searchable database of published miRNA sequences and annotation. Each entry in the miRBase Sequence database represents a predicted hairpin portion of a miRNA transcript (termed mir in the database), with information on the location and sequence of the mature miRNA sequence (termed miR). Both hairpin and mature sequences are available for searching and browsing, and entries can also be retrieved by name, keyword, references and annotation. All sequence and annotation data are also available for download. In October 2018, miRbase version 22.1 was released. This is the current version [Please check].

The term “isomiR” (or “miRNA isoform”), as used herein, refers to a miRNA that varies slightly in sequence, which results from variations in the cleavage site during miRNA biogenesis. In particular, imprecise cleavage of Drosha and Dicer or the turnover of miRNAs can result in miRNAs that are heterogeneous in length and/or sequence. IsomiRs (miRNA isoforms) can be divided into three main categories: 3′ isomiRs (trimmed or addition of one or more nucleotides at the 3′ position), 5′isomiRs (trimmed or addition of one or more nucleotides at the 5′ position), and polymorphic isomiRs (some nucleotides within the sequence are different from the wild type mature miRNA sequence). It could be envisioned that the increased expression of miRNA variants, or individual isomiRs, lead to the loss or weakening of the function of the corresponding wild-type mature miRNA or result in the regulation of a different transcriptome. Recent studies suggest that isomiRs probably play vital roles in a variety of cancers, tissues, and cell types. The detection of miRNAs as well as isomiRs is, thus, absolutely required to accurately reflect the underlying biological situation and to make the right diagnostic and treatment decisions.

RNAs may be isolated from samples using any of a variety of procedures known in the art, for example, the Applied Biosystems ABI Prism® 6100 Nucleic Acid PrepStation (Life Technologies, Foster City, CA) and the ABI Prism® 6700 Automated Nucleic Acid Workstation (Life Technologies, Foster City, CA), Ambion® mirVana™ RNA isolation kit (Life Technologies, Austin, TX), PAXgene Blood RNA Kit (Qiagen, Hilden, Germany) and the like.

The term “total RNA”, as used herein, relates to the isolated RNA (comprising the small (non-coding) RNA, in particular the RNA molecules as described herein) present in a blood sample. Preferably, the total RNA contains a small (non-coding) RNA fraction or a small (no-coding) RNA enriched fraction. For example, the total RNA (comprising the small (non-coding) RNA fraction or the small (non-coding) RNA enriched fraction) is obtained by lysis (e.g. Trizol) of the blood cells in the blood cell preparation, followed by RNA purification e.g. by phenol/chloroform extraction and/or separation-based techniques (e.g. glass fiber filter column, silica-membrane column). Examples of kits for RNA isolation and purification include the miRNeasy Kits (Qiagen), PAXgene Blood miRNA Kit (Qiagen), mirVana PARIS Kit (Life Technologies), PARIS Kit (Life Technologies), Tempus Spin RNA Isolation Kit (Life Technologies).

The term “lung cancer”, as used herein, refers to a disease which consists of uncontrolled cell growth in tissues of the lung. This growth may lead to metastasis, which is the invasion of adjacent tissue and infiltration beyond the lungs. The vast majority of primary lung cancers are carcinomas of the lung, derived from epithelial cells. Lung cancer is the most common cause of cancer-related death in men and women. The most common symptoms are shortness of breath, coughing (including coughing up blood), and weight loss.

Lung cancer can be categorized as lung cancer of stage I, II, III, or IV. In particular, lung cancer can be staged as follows:
Stage I means that the cancer is small. It hasn't spread to the lymph nodes or other distant organs. Stage I belongs to the early lung cancer stages. Stage I can be divided into IA and IB.
Stage IA means the cancer is 3 cm or smaller.
Stage IB means the cancer is between 3 cm and 4 cm. It might also be growing into structures such as: the main airway of the lung (main bronchus) or the membrane covering the lung (visceral pleura).
Stage II still belongs to the early lung cancer stages. Stage II can be divided into stage IIA and IIB. Part of the affected lung might have collapsed.
Stage IIA means that the cancer is between 4 cm and 5 cm in size but there are no cancer cells in any lymph nodes.
Stage IIB means that the cancer is up to 5 cm in size and there are cancer cells in the lymph nodes close to the affected lung. Alternatively, it is between 5 cm and 7 cm but there are no cancer cells in any lymph nodes. Alternatively, the cancer is not in any lymph nodes but has spread into one or more of the following areas: the chest wall (ribs, muscle or skin), the nerve close to the lung (the phrenic nerve), or the layers that cover the heart (mediastinal pleura and parietal pericardium). Alternatively, the cancer is less than 7 cm but there is more than one tumor in the same lobe of the lung.
Stage III can be divided into stage IIIA, IIIB and IIIC. It is sometimes called locally advanced lung cancer.

In stage IIIA, the cancer is up to 5 cm in size and has spread to the lymph nodes in the center of the chest on the same side as the tumor. Alternatively, the cancer it is between 5 cm and 7 cm and there is more than one tumor in the same lobe of the lung. Alternatively, the cancer has spread into one or more of the following areas just outside the lung: the chest wall (ribs, muscle or skin), the nerve close to the lung (the phrenic nerve), the layers that cover the heart (mediastinal pleura and parietal pericardium), or lymph nodes in the lung or close to the lung. Alternatively, the cancer is larger than 7 cm. It hasn't spread into lymph nodes but has spread into one or more of the following areas: the muscle under the lung (diaphragm), the center area of the chest (mediastinum), the heart, a main blood vessel, the wind pipe (trachea), the nerve that goes to the voice box (larynx), the food pipe (oesophagus), a spinal bone, or the area where the wind pipe divides (the carina). Alternatively, the cancer is in more than one lobe of the same lung and there might also be cancer cells in lymph nodes close to the affected lung.

Stage IIIB can also mean different things. The cancer is less than 5 cm and has spread into lymph nodes in one of these places: the opposite side of the chest from the affected lung, the neck, or above the collarbone. Alternatively, the cancer is between 5 cm to 7 cm and has spread into lymph nodes in the center of the chest. Alternatively, the cancer is any size, has spread into lymph nodes in the center of the chest, and has spread into one or more of the following areas: the chest wall, the muscle under the lung (diaphragm), or the layers that cover the heart (mediastinal pleura and parietal pericardium). Alternatively, the cancer has spread into the lymph nodes in the center of the chest. The lung tumor is more than 7 cm or it has spread into a major structure in your chest such as: the heart, the wind pipe (trachea), the food pipe (oesophagus), or a main blood vessel.

Stage IIIC means the cancer is between 5c m and 7 cm in size or has spread into one or more of the following: the nerve close to the lung (phrenic nerve) or the covering of the heart (parietal pericardium) and it has spread into lymph nodes: in the center of the chest on the opposite side from the affected lung or at the top of the lung on the same side or opposite side or above the collar bone. Alternatively, there is more than one tumor in a different lobe of the same lung. Alternatively, stage IIIC can mean the cancer is bigger than 7 cm or it has spread into one of the following: the muscle under the lung (the diaphragm), the center of the chest (mediastinum), the heart, a major blood vessel, the wind pipe (trachea), the nerve going to the voice box (the recurrent laryngeal nerve), the food pipe (oesophagus), a spinal bone, or the area where the windpipe divides (the carina) and it has spread into lymph nodes: in the center of the chest on the opposite side from the affected lung or at the top of the lung on the same side or opposite side or above the collar bone. Alternatively, there are tumors in more than one lobe of the lung.

Stage IV means that the lung cancer has spread. It can be designated as advanced lung cancer. It is divided into stage IVA and IVB.

Stage IVA can mean any of the following: there is cancer in both lungs, the cancer is in the covering of the lung (the pleura) or the covering of the heart (pericardium), or there is fluid around the lungs or the heart that contains cancer cells. Alternatively, it can mean that there is a single area of cancer that has spread outside the chest to a lymph node or to an organ such as the liver or bone.

Stage IVB means that the cancer has spread to several areas in one or more organs.

In one embodiment, the lung cancer is non-small-cell lung carcinoma (NSCLC) or small-cell lung carcinoma (SCLS). In one preferred embodiment, the lung cancer is non-small-cell lung carcinoma (NSCLC). In one more preferred embodiment, the NSCLC is lung adenocarcinoma (LUAD) or lung squamous cell carcinoma (LUSC).

The term “small-cell lung carcinoma (SCLC)”, as used herein, refers to a type of highly malignant cancer that arises within the lungs. Small-cell carcinoma of the lungs usually presents in the central airways and infiltrates the submucosa leading to narrowing of bronchial airways. Common symptoms include cough, dyspnea, weight loss, and debility. Compared to non-small cell carcinoma, small cell carcinoma has a shorter doubling time, higher growth fraction, and earlier development of metastases. SCLC accounts for about 15% of all lung cancers.

Small-cell lung carcinoma can be divided into two clinicopathological stages, termed limited stage (LS) and extensive stage (ES). The stage is generally determined by the presence or absence of metastases, whether or not the tumor appears limited to the thorax, and whether or not the entire tumor burden within the chest can feasibly be encompassed within a single radiotherapy portal. In general, if the tumor is confined to one lung and the lymph nodes close to that lung, the cancer is said to be LS. If cancer has spread beyond that, it is said to be ES.

The term “non-small-cell lung carcinoma (NSCLC)”, as used herein, refers to any type of epithelial lung cancer other than small-cell lung carcinoma (SCLC). NSCLC accounts for about 85% of all lung cancers. As a class, NSCLCs are relatively insensitive to chemotherapy, compared to small-cell lung carcinomas. When possible, they are primarily treated by surgical resection with curative intent, although chemotherapy has been used increasingly both preoperatively (neoadjuvant chemotherapy) and postoperatively (adjuvant chemotherapy). In one embodiment, NSCLC is squamous-cell carcinoma, large-cell carcinoma, or adenocarcinoma.

The term “diagnosing lung cancer”, as used herein, means determining whether a patient shows signs of or suffers from lung cancer.

The term “monitoring a patient suspected of having lung cancer”, as used herein, means determining whether a patient suspected of having lung cancer has developed lung cancer over time.

The term “monitoring a patient having lung cancer”, as used herein, means determining the development of lung cancer in a patient over time, e.g. whether lung cancer worsens in the patient, does not worsen/is stable in the patient, or improves in the patient over time.

The term “(therapeutic) treatment/therapy”, as used herein, relates to any treatment/therapy which improves the health status and/or prolongs (increases) the lifespan of a patient. Said treatment/therapy may eliminate the disease in a patient, arrest, inhibit, or slow the development of a disease in a patient, decrease the frequency or severity of symptoms in a patient, and/or decrease the recurrence in a patient who currently has or who previously has had a disease.

As used herein, the term “lung cancer therapy” refers to any protocol, method, and/or agent that can be used in the prevention, management, treatment, and/or amelioration of lung cancer. In particular, the term “lung cancer therapy”, as used herein, means accomplishing one or more of the following: (i) tumor growth inhibition and/or tumor cell death, (ii) reduction of tumor marker(s), (iii) reduction of tumor lesions and metastases, (iv) reduction of tumor burden as evidenced by imaging studies (e.g. computer tomography (CT), magnetic resonance imaging (MRI), positron emission tomography (PET), etc.), and (v) reduction of tumor burden as evidenced by clinical appraisal or self-report by the patient.

Specifically, lung cancer therapy includes, but is not limited to, drug therapy, supportive therapy, and/or other therapy useful in the prevention, management, treatment, and/or amelioration of lung cancer known to one of skill in the art, such as medical personnel. More specifically, lung cancer therapy includes, but is not limited to, surgery, chemotherapy, targeted therapy, immunotherapy, oncolytic viral therapy, vaccination therapy, radiotherapy, laser therapy, hyperthermia therapy, and administration of a drug, or is a combination thereof, e.g. a combination of chemotherapy and immunotherapy.

The term “patient”, as used herein, refers to any subject for whom it is desired to know whether she or he suffers from lung cancer. In particular, the term “patient”, as used herein, refers to a subject suspected to be affected by lung cancer. The patient may be diagnosed to be affected by lung cancer, i.e. diseased, or may be diagnosed to be not affected by lung cancer, i.e. healthy. The patient may further be a subject who is monitored to determine whether lung cancer developed/further developed in the subject over time or not. In particular, the patient may be monitored in form of a longitudinal monitoring. Thus, the course of lung cancer (particularly after or during treatment) may be observed in the patient over time. The patient may also be retested for lung cancer and may be diagnosed as having developed lung cancer over time or not.

It should be noted that a patient that is diagnosed as being healthy, i.e. not suffering from lung cancer, may possibly suffer from another disease or condition not tested/known.

The patient may be any mammal, including both a human and another mammal, e.g. an animal such as a rabbit, mouse, rat, or monkey. Human individuals are particularly preferred.

The term “(control) subject”, as used herein, refers to a subject known to be not affected by lung cancer (negative control), i.e. healthy. The term “(control) subject”, as used herein, also refers to a subject known to be affected by lung cancer (positive control), i.e. diseased.

It should be noted that a (control) subject which is known to be healthy, i.e. not suffering from cancer, may possibly suffer from another disease or condition not tested/known.

The (control) subject may be any mammal, including both a human and another mammal, e.g. an animal such as a rabbit, mouse, rat, or monkey. Human healthy individuals are particularly preferred.

The term “blood sample”, as used herein, encompasses whole blood or a blood fraction. Preferably, the blood fraction is selected from the group consisting of a blood cell fraction, plasma, and serum. More preferably, the blood cell fraction encompasses/comprises erythrocytes, leukocytes, and/or thrombocytes. Even more preferably, the blood cell fraction is a fraction of leukocytes or a mixture of erythrocytes, leukocytes, and thrombocytes.

It is preferred that the blood sample has a volume of between 0.01 and 20 ml, more preferably of between 0.1 and 10 ml, even more preferably of between 0.5 and 8 ml and most preferably of between 1 and 5 ml.

Said blood sample may be provided by removing blood from a patient or (control) subject, but may also be provided by using a previously isolated sample. For example, a blood sample may be taken from a patient or (control) subject by conventional blood collection techniques.

The blood sample may further be obtained from a patient or (control) subject prior to the initiation of a therapeutic treatment, during the therapeutic treatment, and/or after the therapeutic treatment. If the blood sample is obtained from at least one (control) subject, e.g. from at least 1, 2, 3, 4, 5, 6, 7, 8, 9, 10, 20, 30, 40, 50, 60, 70, 80, 90, 100, 150, 200, 250, 300, 400, 500, or 1,000 (control) subject(s), it is designated as “reference blood sample”. Preferably, the reference blood sample is from the same source than the blood sample of the patient to be tested, e.g. both are whole blood samples or blood cell fractions. It is further preferred that both are from the same species, e.g. from a human. It is also (alternatively or additionally) preferred that the measurements of the reference blood sample of the (control) subject and the blood sample of the patient to be tested are identical, e.g. both have an identical volume. It is particularly preferred that the reference blood sample and the blood sample are from (control) subjects/patients of the same sex and similar age.

The whole blood sample may be collected by means of a blood collection tube. It is, for example, collected in a PAXgene Blood RNA tube, in a Tempus Blood RNA tube, in an EDTA-tube, in a Na-citrate tube, Heparin-tube, or in an ACD-tube (Acid citrate dextrose). Alternatively, the whole blood sample may be collected in a blood collection tube containing cell-free nucleic acid stabilizing chemical agents, such as glutaraldehyde, formaldehyde, or similar (e.g. Streck cfRNA BCT tube, Streck cfDNA BCT tube), and others, or cellular crowding agents, such as polyethyleneglycol (PEG) (e.g. Norgen cfDNA/cfRNA preservation tube), and others.

The blood sample, in particular whole blood sample, as used herein, may also be collected by means of a bloodspot technique, e.g. using a Mitra Microsampling Device. This technique requires smaller sample volumes, typically 45-60 μl for humans or less. For example, the whole blood may be extracted from the patient via a finger prick with a needle or lancet. Thus, the whole blood sample may have the form of a blood drop. Said blood drop is then placed on an absorbent probe, e.g. a hydrophilic polymeric material such as cellulose, which is capable of absorbing the whole blood. Once sampling is complete, the blood spot is dried in air before transferring or mailing to labs for processing. Because the blood is dried, it is not considered hazardous. Thus, no special precautions need be taken in handling or shipping. Once at the analysis site, the desired components, e.g. the RNA molecule as described herein, are extracted from the dried blood spots into a supernatant which is then further analyzed. In this way, the level of the RNA molecule is determined. This technique is suitable for monitoring patients having lung cancer at home (on a home care/home sampling basis) or for screening purposes.

In the methods described herein, the level of the RNA molecule is determined or the levels of the RNA molecules are determined in a blood sample of a patient. The term “level”, as used herein, refers to an amount (measured for example in grams, mole, or ion counts) or concentration (e.g. absolute or relative concentration, e.g. reads per million (RPM) or NGS counts) of the RNA molecule. The term “level”, as used herein, also comprises scaled, normalized, or scaled and normalized amounts or values (e.g. RPM). In particular, the level of the RNA molecule is determined by sequencing, preferably next generation sequencing (e.g. ABI SOLID, Illumina Genome Analyzer, Roche 454 GS FL, BGISEQ), nucleic acid hybridization (e.g. microarray or beads), nucleic acid amplification (e.g. PCR, RT-PCR, qRT-PCR, or high-throughput RT-PCR), polymerase extension, mass spectrometry, flow cytometry (e.g. LUMINEX), or any combination thereof. Specifically, the level of the RNA molecule is the expression level of said RNA molecule.

Those of skill in the art will appreciate that, in many embodiments described herein, the determined RNA molecule level is compared with an appropriate RNA molecule “reference level”. Specifically, the level of the RNA molecule is compared to a reference level of said RNA molecule. More specifically, the reference level of an RNA molecule is determined in a blood sample of (control) subjects. Even more specifically, the reference level is determined empirically by measuring a number of reference blood samples from subjects known to suffer from lung cancer or by measuring a number of reference blood samples from subjects known to not suffer from lung cancer. Typically, as would be understood by those skilled in the art, the reference level is determined under conditions comparable to those utilized to determine or analyze the RNA molecule level in a blood sample of a patient.

In the methods described herein, the level of the at least one RNA molecule is compared to a reference level of said at least one RNA molecule. Said reference level is the level determined by measuring a reference blood sample. For example, if the level of the RNA molecule having a nucleotide sequence according to SEQ ID NO: 1 is determined in a blood sample of a patient, this level is compared to a reference level of the RNA molecule having a nucleotide sequence according to SEQ ID NO: 1 determined in a reference blood sample.

Alternatively, in the methods described herein, the levels of the at least two RNA molecules are compared to reference levels of said at least two RNA molecules. Said reference levels are the levels determined by measuring a reference blood sample. For example, if the level of the RNA molecule having a nucleotide sequence according to SEQ ID NO: 1 and the level of the RNA molecule having a nucleotide sequence according to SEQ ID NO: 2 are determined in a blood sample of a patient, both levels are compared to the respective reference levels, i.e. the level of the RNA molecule having a nucleotide sequence according to SEQ ID NO: 1 is compared to the reference level of the RNA molecule having a nucleotide sequence according to SEQ ID NO: 1 and the level of the RNA molecule having a nucleotide sequence according to SEQ ID NO: 2 is compared to the reference level of the RNA molecule having a nucleotide sequence according to SEQ ID NO: 2 determined in a reference blood sample.

The reference level may also be a cut-off or threshold level. Typically, a cut-off or threshold level can be determined experimentally, empirically, or theoretically. A cut-off or threshold level can also be arbitrarily selected based upon the existing experimental and/or clinical conditions, as would be recognized by a person of ordinary skilled in the art. The cut-off or threshold level must be determined in order to obtain the optimal sensitivity and specificity according to the function of the test and the benefit/risk balance (clinical consequences of false positive and false negative). The cut-off or threshold level can also be a cut-off or threshold score, or can also be converted into a cut-off or threshold score.

Typically, the optimal sensitivity and specificity (and so the cut-off or threshold level/score) can be determined using a Receiver Operating Characteristic (ROC) curve based on experimental data. For example, after determining RNA molecule levels in a reference group, one can use algorithmic analysis for the statistic treatment of the measured RNA molecule levels in samples to be tested and, thus, obtain a classification standard having significance for sample classification. The ROC curve is mainly used for clinical biomarker tests. The ROC curve is a comprehensive indicator that reflects the continuous variables of true positive rate (sensitivity) and false positive rate (1-specificity). It reveals the relationship between sensitivity and specificity with the image composition method. A series of different cut-off or threshold levels/scores are set as continuous variables to calculate a series of sensitivity and specificity values. Then sensitivity is used as the vertical coordinate and specificity is used as the horizontal coordinate to draw a curve.

An important measure is the area under the ROC curve value (AUC value). If this area is equal to 1.0 then the test is 100% accurate (has an accuracy of 100%) because both the sensitivity and specificity are 1.0 so there are no false positives and no false negatives. A test that cannot discriminate has an AUC value of 0.5. AUC values are typically between 0.5 and 1.0, but also AUC values below 0.5 can—according to information theory—be as good, if the result is interpreted inversely. Therefore, according to the present invention, an AUC-value close to 1 (e.g. 0.95) represents the same good measure for a clinical test as an AUC-value close to 0 (e.g. 0.05). In other words, when the AUC value is >0.5, the diagnosed result gets better and better as the AUC value approaches 1. Alternatively, when the AUC value is <0.5, the diagnosed result gets better and better as the AUC value approaches 0.

The term “classifier”, as used herein, refers to a predictive model which allows to distinguish between or characterize samples by classifying a given sample into a predetermined class based on certain characteristics of said sample. For example, a classifier is capable of predicting with a relatively high sensitivity and specificity if a patient of an unknown diagnosis belongs to one of two given classes; i.e. a class of diseased subjects or a class of healthy subjects. The output may be given as a probability of belonging to either class of between 0-1. Specifically, a classifier allows to distinguish patients suffering from lung cancer from patients not suffering from lung cancer, i.e. being healthy with respect to lung cancer.

The term “point-of-care testing (POCT)”, as used herein, refers to a medical diagnostic testing at or near the point of care that is the time and place of individual care. This contrasts with the historical pattern in which testing was wholly or mostly confined to the medical laboratory, which entailed sending off specimens away from the point of care and then waiting hours or days to learn the results, during which time care must continue without the desired information. Point-of-care tests are simple medical tests that can be performed at the bedside. The driving notion behind POCT is to bring the test conveniently and immediately to the individual to be tested. This increases the likelihood that the individual, physician, and care team will receive the results quicker, which allows for immediate clinical management decisions to be made. POCT is often accomplished through the use of transportable, portable, and handheld instruments and test kits. Small bench analyzers or fixed equipment can also be used when a handheld device is not available—the goal is to collect the specimen and obtain the results in a very short period of time at or near the location of the individual so that the treatment plan can be adjusted as necessary before the individual leaves the hospital.

In the context of the present invention, the term “kit of parts (in short: kit)” is understood to be any combination of at least some of the components identified herein, which are combined, coexisting spatially, to a functional unit, and which can contain further components.

### EMBODIMENTS OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

The present inventors identified new RNA molecules which are significantly deregulated in lung cancer patients compared to healthy controls. It has turned out that signatures of these new RNA molecules are of high diagnostic relevance for lung cancer determination. These new RNA molecules can, thus, be used as biomarkers for lung cancer diagnosis and as biomarkers for lung cancer monitoring.

Thus, in a first aspect, the present invention relates to a (an) (in vitro) method of diagnosing/detecting/determining lung cancer in a patient comprising the step of:
(i) determining the level of at least one RNA molecule in a blood sample from a patient, wherein the at least one RNA molecule comprises/has a nucleotide sequence according to SEQ ID NO: 1, is a fragment thereof, or has a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto,
(ii) determining the levels of at least two RNA molecules in a blood sample from a patient, wherein the at least two RNA molecules comprise a first RNA molecule and a second RNA molecule,
   wherein the first RNA molecule comprises/has a nucleotide sequence according to SEQ ID NO: 1, is a fragment thereof, or has a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and the second RNA molecule comprises/has a nucleotide sequence according to SEQ ID NO: 2, is a fragment thereof, or has a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto, or
(iii) determining the levels of at least two RNA molecules in a blood sample from a patient, wherein the at least two RNA molecules comprise a first RNA molecule and a second RNA molecule,
   wherein the first RNA molecule comprises a nucleotide sequence according to SEQ ID NO: 2, is a fragment thereof, or has a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and the second RNA molecule comprises a nucleotide sequence according to SEQ ID NO: 3, is a fragment thereof, or has a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Preferably,
the first RNA molecule comprises a nucleotide sequence according to SEQ ID NO: 1 and the second RNA molecule comprises a nucleotide sequence according to SEQ ID NO: 2, or
the first RNA molecule comprises a nucleotide sequence according to SEQ ID NO: 2 and the second RNA molecule comprises a nucleotide sequence according to SEQ ID NO: 3.

Especially, the at least two RNA molecules are comprised in a set. In other words, the at least two RNA molecules represent a signature. The signature can encompass, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 RNA molecules, specifically 2, 5, 6, 10, 18, 38, or 54 RNA molecules (RNA molecule signature).

In embodiment (i),
the level of at least one further RNA molecule selected from the group consisting of an RNA molecule comprising/having a nucleotide sequence according to SEQ ID NO: 2 to SEQ ID NO: 71, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto is determined, or
the level of at least one further RNA molecule selected from the group consisting of an RNA molecule comprising/having a nucleotide sequence according to SEQ ID NO: 2 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto is determined.

Thus, the level of the RNA molecule comprising a nucleotide sequence according to SEQ ID NO: 1, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and the level of at least one further RNA molecule selected from the group consisting of an RNA molecule comprising a nucleotide sequence according to SEQ ID NO: 2 to SEQ ID NO: 71, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are determined in the blood sample from the patient, or
the level of at least one further RNA molecule selected from the group consisting of an RNA molecule comprising a nucleotide sequence according to SEQ ID NO: 2 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are determined in the blood sample from the patient.

Specifically, the level of the RNA molecule comprising
(i) a nucleotide sequence according to SEQ ID NO: 1 to SEQ ID NO: 71 or SEQ ID NO: 1 to SEQ ID NO: 79,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iii) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii) is determined in the blood sample.

In embodiment (ii),
the level of at least one further RNA molecule selected from the group consisting of an RNA molecule comprising/having a nucleotide sequence according to SEQ ID NO: 3 to SEQ ID NO: 71, a fragment thereof, and a sequence having at least 80% sequence identity thereto is determined, or
the level of at least one further RNA molecule selected from the group consisting of an RNA molecule comprising a nucleotide sequence according to SEQ ID NO: 3 to SEQ ID NO: 79, a fragment thereof, and a sequence having at least 80% sequence identity thereto is determined. Thus, the level of the RNA molecule comprising a nucleotide sequence according to SEQ ID NO: 1, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto as well as the level of the RNA molecule comprising a nucleotide sequence according to SEQ ID NO: 2, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and
the level of at least one further RNA molecule selected from the group consisting of an RNA molecule comprising a nucleotide sequence according to SEQ ID NO: 3 to SEQ ID NO: 71, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are determined in the blood sample from the patient, or
the level of at least one further RNA molecule selected from the group consisting of an RNA molecule comprising a nucleotide sequence according to SEQ ID NO: 3 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are determined in the blood sample from the patient.

Specifically, the level of the RNA molecule comprising
(i) a nucleotide sequence according to SEQ ID NO: 1 to SEQ ID NO: 71 or SEQ ID NO: 1 to SEQ ID NO: 79,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iii) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii) is determined in the blood sample.

Preferably,
(i) the level of at least one further RNA molecule selected from the group consisting of an RNA molecule comprising a nucleotide sequence according to SEQ ID NO: 19, SEQ ID NO: 35, SEQ ID NO: 39, SEQ ID NO: 48, a fragment thereof, and a sequence having at least 80% sequence identity thereto is determined,
(ii) the level of at least one further RNA molecule selected from the group consisting of an RNA molecule comprising a nucleotide sequence according to SEQ ID NO: 23, SEQ ID NO: 39, SEQ ID NO: 48, a fragment thereof, and a sequence having at least 80% sequence identity thereto is determined,
(iii) the level of at least one further RNA molecule selected from the group consisting of an RNA molecule comprising a nucleotide sequence according to SEQ ID NO: 6, SEQ ID NO: 7, SEQ ID NO: 9, a fragment thereof, and a sequence having at least 80% sequence identity thereto is determined,
(iv) the level of at least one further RNA molecule selected from the group consisting of an RNA molecule comprising a nucleotide sequence according to SEQ ID NO: 6, SEQ ID NO: 7, SEQ ID NO: 11, SEQ ID NO: 18, a fragment thereof, and a sequence having at least 80% sequence identity thereto is determined,
(v) the level of at least one further RNA molecule selected from the group consisting of an RNA molecule comprising a nucleotide sequence according to SEQ ID NO: 19, SEQ ID NO: 35, SEQ ID NO: 38, SEQ ID NO: 39, SEQ ID NO: 40, SEQ ID NO: 43, SEQ ID NO: 48, SEQ ID NO: 49, a fragment thereof, and a sequence having at least 80% sequence identity thereto is determined,
(vi) the level of at least one further RNA molecule selected from the group consisting of an RNA molecule comprising a nucleotide sequence according to SEQ ID NO: 17, SEQ ID NO: 23, SEQ ID NO: 35, SEQ ID NO: 39, SEQ ID NO: 45, SEQ ID NO: 48, SEQ ID NO: 52, SEQ ID NO: 53, a fragment thereof, and a sequence having at least 80% sequence identity thereto is determined,
(vii) the level of at least one further RNA molecule selected from the group consisting of an RNA molecule comprising a nucleotide sequence according to SEQ ID NO: 6, SEQ ID NO: 7, SEQ ID NO: 9, SEQ ID NO: 19, SEQ ID NO: 25, SEQ ID NO: 29, SEQ ID NO: 30, SEQ ID NO: 38, a fragment thereof, and a sequence having at least 80% sequence identity thereto is determined,
(viii) the level of at least one further RNA molecule selected from the group consisting of an RNA molecule comprising a nucleotide sequence according to SEQ ID NO: 3, SEQ ID NO: 6, SEQ ID NO: 7, SEQ ID NO: 11, SEQ ID NO: 12, SEQ ID NO: 14, SEQ ID NO: 18, SEQ ID NO: 28, a fragment thereof, and a sequence having at least 80% sequence identity thereto is determined,
(ix) the level of at least one further RNA molecule selected from the group consisting of an RNA molecule comprising a nucleotide sequence according to SEQ ID NO: 3 to SEQ ID NO: 54, a fragment thereof, and a sequence having at least 80% sequence identity thereto is determined, or
(x) the level of at least one further RNA molecule selected from the group consisting of an RNA molecule comprising a nucleotide sequence according to SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, a fragment thereof, and a sequence having at least 80% sequence identity thereto is determined.

More preferably, the levels of the RNA molecules comprising a nucleotide sequence according to
(i) SEQ ID NO: 1, SEQ ID NO: 2, SEQ ID NO: 19, SEQ ID NO: 35, SEQ ID NO: 39, and SEQ ID NO: 48,
(ii) SEQ ID NO: 1, SEQ ID NO: 2, SEQ ID NO: 23, SEQ ID NO: 39, and SEQ ID NO: 48,
(iii) SEQ ID NO: 1, SEQ ID NO: 2, SEQ ID NO: 6, SEQ ID NO: 7, and SEQ ID NO: 9,
(iv) SEQ ID NO: 1, SEQ ID NO: 2, SEQ ID NO: 6, SEQ ID NO: 7, SEQ ID NO: 11, and SEQ ID NO: 18,
(v) SEQ ID NO: 1, SEQ ID NO: 2, SEQ ID NO: 19, SEQ ID NO: 35, SEQ ID NO: 38, SEQ ID NO: 39, SEQ ID NO: 40, SEQ ID NO: 43, SEQ ID NO: 48, and SEQ ID NO: 49,
(vi) SEQ ID NO: 1, SEQ ID NO: 2, SEQ ID NO: 17, SEQ ID NO: 23, SEQ ID NO: 35, SEQ ID NO: 39, SEQ ID NO: 45, SEQ ID NO: 48, SEQ ID NO: 52, and SEQ ID NO: 53,
(vii) SEQ ID NO: 1, SEQ ID NO: 2, SEQ ID NO: 6, SEQ ID NO: 7, SEQ ID NO: 9, SEQ ID NO: 19, SEQ ID NO: 25, SEQ ID NO: 29, SEQ ID NO: 30, and SEQ ID NO: 38,
(viii) SEQ ID NO: 1, SEQ ID NO: 2, SEQ ID NO: 3, SEQ ID NO: 6, SEQ ID NO: 7, SEQ ID NO: 11, SEQ ID NO: 12, SEQ ID NO: 14, SEQ ID NO: 18, and SEQ ID NO: 28,
(ix) SEQ ID NO: 1 to SEQ ID NO: 54, or
(x) SEQ ID NO: 1, SEQ ID NO: 2, SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, and SEQ ID NO: 71
are determined.

In embodiment (iii), the level of at least one further RNA molecule selected from the group consisting of an RNA molecule comprising/having a nucleotide sequence according to SEQ ID NO: 1, SEQ ID NO: 4 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto is determined.

Thus, the level of the RNA molecule comprising a nucleotide sequence according to SEQ ID NO: 2, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto as well as the level of the RNA molecule comprising a nucleotide sequence according to SEQ ID NO: 3, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and the level of at least one further RNA molecule selected from the group consisting of an RNA molecule comprising a nucleotide sequence according to SEQ ID NO: 1, SEQ ID NO: 4 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are determined in the blood sample from the patient.

Specifically, the level of the RNA molecule comprising
(i) a nucleotide sequence according to SEQ ID NO: 1 to SEQ ID NO: 79,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iii) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii) is determined in the blood sample.

Preferably,
(i) the level of at least one further RNA molecule selected from the group consisting of an RNA molecule comprising a nucleotide sequence according to SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72 to SEQ ID NO: 79, a fragment thereof, and a sequence having at least 80% sequence identity thereto is determined, or
(ii) the level of at least one further RNA molecule selected from the group consisting of an RNA molecule comprising a nucleotide sequence according to SEQ ID NO: 7, SEQ ID NO: 17, SEQ ID NO: 18, SEQ ID NO: 75, a fragment thereof, and a sequence having at least 80% sequence identity thereto is determined.

More preferably, the levels of the RNA molecules comprising a nucleotide sequence according to
(i) SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, and SEQ ID NO: 79, or
(ii) SEQ ID NO: 2, SEQ ID NO: 3, SEQ ID NO: 7, SEQ ID NO: 17, SEQ ID NO: 18, and SEQ ID NO: 75 are determined.

The above (6) signature encompassing RNA molecules comprising a nucleotide sequence according to SEQ ID NO: 2, SEQ ID NO: 3, SEQ ID NO: 7, SEQ ID NO: 17, SEQ ID NO: 18, and SEQ ID NO: 75 was obtained in blood samples sampled/collected with a Mitra Microsampling Device. The RNA molecules were isolated from dry blood spots (DBS) and subsequently used for molecular biological analysis, specifically for determining the levels of these RNA molecules.

In preferred embodiment (i), the level of the at least one RNA molecule is compared to a reference. Particularly, the level of the at least one RNA molecule is compared to a reference level of said RNA molecule.

The above comparison allows to diagnose lung cancer in a patient, in particular in a patient suspected of suffering from lung cancer. The patient may be diagnosed as suffering from lung cancer, i.e. being diseased, or as not suffering from lung cancer, i.e. being healthy.

The reference, such as the reference level, may be any reference, such as reference level, which allows to determine whether the patient suffers from lung cancer or not. The reference level may be obtained from (control) subjects (i.e. subjects different from the patient to be tested such as healthy subjects and/or subjects known to have lung cancer) or from the same patient. In the latter case, the patient may be retested for lung cancer, e.g. in the form of a longitudinal monitoring. It may be determined that the patient is now affected by lung cancer or still not affected by lung cancer.

More particularly, the reference level is the level of the at least one RNA molecule determined empirically by measuring a number of reference blood samples from subjects known to be healthy (not suffering from lung cancer), and/or from subjects known to suffer from lung cancer.

For example,
the reference level is determined from at least 2, at least 10, at least 50, at least 100, at least 200, at least 300, at least 400, at least 500, at least 1000, at least 1500, at least 2000 at least 5000 reference samples from healthy subjects, and/or
the reference level is determined from at least 2, at least 10, at least 50, at least 100, at least 200, at least 300, at least 400, at least 500, at least 1000, at least 1500, at least 2000 at least 5000 reference samples from subjects known to suffer from lung cancer.

Specifically, the reference level is an average reference level. It is determined by measuring reference levels of control subjects (e.g. healthy subjects or subjects having lung cancer/known to have lung cancer) and calculating the “average” value (e.g. mean, median or modal value) thereof. It is preferred that the reference blood sample is from the same source (e.g. blood cells, serum, or plasma) than the blood sample isolated from the patient to be tested. It is further preferred that the reference level is obtained from control subjects (e.g. healthy subjects or subjects having lung cancer/known to have lung cancer) of the same gender (e.g. female or male) and/or of a similar age/phase of life (e.g. adults or elderly) than the patient to be tested. Especially, the reference level represents an average value of the RNA molecule in a healthy population and/or the reference level represents an average value of the RNA molecule in a population of subjects having lung cancer/known to have lung cancer.

Preferably, the reference level is the level of the at least one RNA molecule determined empirically by measuring a number of reference blood samples from subjects known to be healthy (not suffering from lung cancer), wherein
the level of the RNA molecule selected from the group consisting of SEQ ID NO: 1, SEQ ID NO: 2, SEQ ID NO: 3, SEQ ID NO: 6, SEQ ID NO: 7, SEQ ID NO: 8, SEQ ID NO: 9, SEQ ID NO: 11, SEQ ID NO: 12, SEQ ID NO: 15, SEQ ID NO: 18, SEQ ID NO: 19, SEQ ID NO: 20, SEQ ID NO: 23, SEQ ID NO: 24, SEQ ID NO: 33, SEQ ID NO: 34, SEQ ID NO: 36, SEQ ID NO: 38, SEQ ID NO: 41, SEQ ID NO: 43, SEQ ID NO: 44, SEQ ID NO: 45, SEQ ID NO: 46, SEQ ID NO: 49, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 62, SEQ ID NO: 69, SEQ ID NO: 72, SEQ ID NO: 73, SEQ ID NO: 77, and/or SEQ ID NO: 78 above the reference level indicates that the patient suffers from lung cancer, and/or
the level of the RNA molecule selected from the group consisting of SEQ ID NO: 4, SEQ ID NO: 10, SEQ ID NO: 14, SEQ ID NO: 16, SEQ ID NO: 17, SEQ ID NO: 21, SEQ ID NO: 22, SEQ ID NO: 25, SEQ ID NO: 26, SEQ ID NO: 28, SEQ ID NO: 29, SEQ ID NO: 30, SEQ ID NO: 31, SEQ ID NO: 32, SEQ ID NO: 35, SEQ ID NO: 39, SEQ ID NO: 40, SEQ ID NO: 42, SEQ ID NO: 47, SEQ ID NO: 48, SEQ ID NO: 52, SEQ ID NO: 61, SEQ ID NO: 63, SEQ ID NO: 64, SEQ ID NO: 65, SEQ ID NO: 66, SEQ ID NO: 67, SEQ ID NO: 68, SEQ ID NO: 70, SEQ ID NO: 71, SEQ ID NO: 74, SEQ ID NO: 75, SEQ ID NO: 76, and/or SEQ ID NO: 79 below the reference level indicates that the patient suffers from lung cancer.

Alternatively or additionally, the reference level is the level of the at least one RNA molecule determined empirically by measuring a number of reference blood samples from subjects known to suffer from lung cancer, wherein
the level of the RNA molecule selected from the group consisting of SEQ ID NO: 1, SEQ ID NO: 2, SEQ ID NO: 3, SEQ ID NO: 6, SEQ ID NO: 7, SEQ ID NO: 8, SEQ ID NO: 9, SEQ ID NO: 11, SEQ ID NO: 12, SEQ ID NO: 15, SEQ ID NO: 18, SEQ ID NO: 19, SEQ ID NO: 20, SEQ ID NO: 23, SEQ ID NO: 24, SEQ ID NO: 33, SEQ ID NO: 34, SEQ ID NO: 36, SEQ ID NO: 38, SEQ ID NO: 41, SEQ ID NO: 43, SEQ ID NO: 44, SEQ ID NO: 45, SEQ ID NO: 46, SEQ ID NO: 49, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 62, SEQ ID NO: 69, SEQ ID NO: 72, SEQ ID NO: 73, SEQ ID NO: 77, and/or SEQ ID NO: 78 below the reference level indicates that the patient is healthy (does not suffer from lung cancer), and/or
the level of the RNA molecule selected from the group consisting of SEQ ID NO: 4, SEQ ID NO: 10, SEQ ID NO: 14, SEQ ID NO: 16, SEQ ID NO: 17, SEQ ID NO: 21, SEQ ID NO: 22, SEQ ID NO: 25, SEQ ID NO: 26, SEQ ID NO: 28, SEQ ID NO: 29, SEQ ID NO: 30, SEQ ID NO: 31, SEQ ID NO: 32, SEQ ID NO: 35, SEQ ID NO: 39, SEQ ID NO: 40, SEQ ID NO: 42, SEQ ID NO: 47, SEQ ID NO: 48, SEQ ID NO: 52, SEQ ID NO: 61, SEQ ID NO: 63, SEQ ID NO: 64, SEQ ID NO: 65, SEQ ID NO: 66, SEQ ID NO: 67, SEQ ID NO: 68, SEQ ID NO: 70, SEQ ID NO: 71, SEQ ID NO: 74, SEQ ID NO: 75, SEQ ID NO: 76, and/or SEQ ID NO: 79 above the reference level indicates that the patient is healthy (does not suffer from lung cancer).

Specifically, the level of the RNA molecule is at least 0.6-fold or 0.7-fold, more specifically at least 0.8-fold or 0.9-fold, even more specifically at least 1.2-fold or 1.5-fold, and still even more specifically at least 2.0-fold or 3.0-fold above/below the reference level. For example, the level of the RNA molecule is at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold above/below the reference level.

In preferred embodiment (ii), the levels of the at least two RNA molecules are compared to references. Particularly, the levels of the at least two RNA molecules are compared to reference levels of said RNA molecules.

The above comparison allows to diagnose lung cancer in a patient, in particular in a patient suspected of suffering from lung cancer. The patient may be diagnosed as suffering from lung cancer, i.e. being diseased, or as not suffering from lung cancer, i.e. being healthy.

The references, such as the reference levels, may be references, such as reference levels, which allow to determine whether the patient suffers from lung cancer or not. The reference levels may be obtained from (control) subjects (i.e. subjects different from the patient to be tested such as healthy subjects and/or subjects known to have lung cancer) or from the same patient. In the latter case, the patient may be retested for lung cancer, e.g. in the form of a longitudinal monitoring. It may be determined that the patient is now affected by lung cancer or still not affected by lung cancer.

More particularly, the reference levels are the levels of the at least two RNA molecules determined empirically by measuring a number of reference blood samples
from subjects known to be healthy (not suffering from lung cancer), and/or
from subjects known to suffer from lung cancer.

For example,
the reference levels are determined from at least 2, at least 10, at least 50, at least 100, at least 200, at least 300, at least 400, at least 500, at least 1000, at least 1500, at least 2000 at least 5000 reference samples from healthy subjects, and/or
the reference levels are determined from at least 2, at least 10, at least 50, at least 100, at least 200, at least 300, at least 400, at least 500, at least 1000, at least 1500, at least 2000 at least 5000 reference samples from subjects known to suffer from lung cancer.

Specifically, the reference levels are average reference levels. They are determined by measuring reference levels of control subjects (e.g. healthy subjects or subjects having lung cancer/known to have lung cancer) and calculating “average” values (e.g. mean, median or modal value) thereof. Especially, the reference levels represent average values of the RNA molecules in a healthy population and/or the reference levels represent average values of the RNA molecules in a population of subjects having lung cancer/known to have lung cancer. In this respect, it should be noted that for each RNA molecule, a level is determined which is then compared to the respective reference level of the same RNA molecule.

Preferably, the reference levels are the levels of the at least two RNA molecules determined empirically by measuring a number of reference blood samples from subjects known to be healthy (not suffering from lung cancer), wherein
the level of the RNA molecule selected from the group consisting of SEQ ID NO: 1, SEQ ID NO: 2, SEQ ID NO: 3, SEQ ID NO: 6, SEQ ID NO: 7, SEQ ID NO: 8, SEQ ID NO: 9, SEQ ID NO: 11, SEQ ID NO: 12, SEQ ID NO: 15, SEQ ID NO: 18, SEQ ID NO: 19, SEQ ID NO: 20, SEQ ID NO: 23, SEQ ID NO: 24, SEQ ID NO: 33, SEQ ID NO: 34, SEQ ID NO: 36, SEQ ID NO: 38, SEQ ID NO: 41, SEQ ID NO: 43, SEQ ID NO: 44, SEQ ID NO: 45, SEQ ID NO: 46, SEQ ID NO: 49, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 62, SEQ ID NO: 69, SEQ ID NO: 72, SEQ ID NO: 73, SEQ ID NO: 77, and/or SEQ ID NO: 78 above the reference level indicates that the patient suffers from lung cancer, and/or
the level of the RNA molecule selected from the group consisting of SEQ ID NO: 4, SEQ ID NO: 10, SEQ ID NO: 14, SEQ ID NO: 16, SEQ ID NO: 17, SEQ ID NO: 21, SEQ ID NO: 22, SEQ ID NO: 25, SEQ ID NO: 26, SEQ ID NO: 28, SEQ ID NO: 29, SEQ ID NO: 30, SEQ ID NO: 31, SEQ ID NO: 32, SEQ ID NO: 35, SEQ ID NO: 39, SEQ ID NO: 40, SEQ ID NO: 42, SEQ ID NO: 47, SEQ ID NO: 48, SEQ ID NO: 52, SEQ ID NO: 61, SEQ ID NO: 63, SEQ ID NO: 64, SEQ ID NO: 65, SEQ ID NO: 66, SEQ ID NO: 67, SEQ ID NO: 68, SEQ ID NO: 70, SEQ ID NO: 71, SEQ ID NO: 74, SEQ ID NO: 75, SEQ ID NO: 76, and/or SEQ ID NO: 79 below the reference level indicates that the patient suffers from lung cancer.

Alternatively or additionally, the reference levels are the levels of the at least two RNA molecules determined empirically by measuring a number of reference blood samples from subjects known to suffer from lung cancer, wherein
the level of the RNA molecule selected from the group consisting of SEQ ID NO: 1, SEQ ID NO: 2, SEQ ID NO: 3, SEQ ID NO: 6, SEQ ID NO: 7, SEQ ID NO: 8, SEQ ID NO: 9, SEQ ID NO: 11, SEQ ID NO: 12, SEQ ID NO: 15, SEQ ID NO: 18, SEQ ID NO: 19, SEQ ID NO: 20, SEQ ID NO: 23, SEQ ID NO: 24, SEQ ID NO: 33, SEQ ID NO: 34, SEQ ID NO: 36, SEQ ID NO: 38, SEQ ID NO: 41, SEQ ID NO: 43, SEQ ID NO: 44, SEQ ID NO: 45, SEQ ID NO: 46, SEQ ID NO: 49, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 62, SEQ ID NO: 69, SEQ ID NO: 72, SEQ ID NO: 73, SEQ ID NO: 77, and/or SEQ ID NO: 78 below the reference level indicates that the patient is healthy (does not suffer from lung cancer), and/or
the level of the RNA molecule selected from the group consisting of SEQ ID NO: 4, SEQ ID NO: 10, SEQ ID NO: 14, SEQ ID NO: 16, SEQ ID NO: 17, SEQ ID NO: 21, SEQ ID NO: 22, SEQ ID NO: 25, SEQ ID NO: 26, SEQ ID NO: 28, SEQ ID NO: 29, SEQ ID NO: 30, SEQ ID NO: 31, SEQ ID NO: 32, SEQ ID NO: 35, SEQ ID NO: 39, SEQ ID NO: 40, SEQ ID NO: 42, SEQ ID NO: 47, SEQ ID NO: 48, SEQ ID NO: 52, SEQ ID NO: 61, SEQ ID NO: 63, SEQ ID NO: 64, SEQ ID NO: 65, SEQ ID NO: 66, SEQ ID NO: 67, SEQ ID NO: 68, SEQ ID NO: 70, SEQ ID NO: 71, SEQ ID NO: 74, SEQ ID NO: 75, SEQ ID NO: 76, and/or SEQ ID NO: 79 above the reference level indicates that the patient is healthy (does not suffer from lung cancer).

Specifically, the level of the RNA molecule is at least 0.6-fold or 0.7-fold, more specifically at least 0.8-fold or 0.9-fold, even more specifically at least 1.2-fold or 1.5-fold, and still even more specifically at least 2.0-fold or 3.0-fold above/below the reference level. For example, the level of the RNA molecule is at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold above/below the reference level.

In preferred embodiment (iii), the levels of the at least two RNA molecules are compared to references. Particularly, the levels of the at least two RNA molecules are compared to reference levels of said RNA molecules.

The above comparison allows to diagnose lung cancer in a patient, in particular in a patient suspected of suffering from lung cancer. The patient may be diagnosed as suffering from lung cancer, i.e. being diseased, or as not suffering from lung cancer, i.e. being healthy.

The references, such as the reference levels, may be references, such as reference levels, which allow to determine whether the patient suffers from lung cancer or not. The reference levels may be obtained from (control) subjects (i.e. subjects different from the patient to be tested such as healthy subjects and/or subjects known to have lung cancer) or from the same patient. In the latter case, the patient may be retested for lung cancer, e.g. in the form of a longitudinal monitoring. It may be determined that the patient is now affected by lung cancer or still not affected by lung cancer.

More particularly, the reference levels are the levels of the at least two RNA molecules determined empirically by measuring a number of reference blood samples from subjects known to be healthy (not suffering from lung cancer), and/or from subjects known to suffer from lung cancer.

For example,
the reference levels are determined from at least 2, at least 10, at least 50, at least 100, at least 200, at least 300, at least 400, at least 500, at least 1000, at least 1500, at least 2000 at least 5000 reference samples from healthy subjects, and/or
the reference levels are determined from at least 2, at least 10, at least 50, at least 100, at least 200, at least 300, at least 400, at least 500, at least 1000, at least 1500, at least 2000 at least 5000 reference samples from subjects known to suffer from lung cancer.

Specifically, the reference levels are average reference levels. They are determined by measuring reference levels of control subjects (e.g. healthy subjects or subjects having lung cancer/known to have lung cancer) and calculating “average” values (e.g. mean, median or modal value) thereof. Especially, the reference levels represent average values of the RNA molecules in a healthy population and/or the reference levels represent average values of the RNA molecules in a population of subjects having lung cancer/known to have lung cancer. In this respect, it should be noted that for each RNA molecule, a level is determined which is then compared to the respective reference level of the same RNA molecule.

Preferably, the reference levels are the levels of the at least two RNA molecules determined empirically by measuring a number of reference blood samples from subjects known to be healthy (not suffering from lung cancer), wherein
the level of the RNA molecule selected from the group consisting of SEQ ID NO: 1, SEQ ID NO: 2, SEQ ID NO: 3, SEQ ID NO: 6, SEQ ID NO: 7, SEQ ID NO: 8, SEQ ID NO: 9, SEQ ID NO: 11, SEQ ID NO: 12, SEQ ID NO: 15, SEQ ID NO: 18, SEQ ID NO: 19, SEQ ID NO: 20, SEQ ID NO: 23, SEQ ID NO: 24, SEQ ID NO: 33, SEQ ID NO: 34, SEQ ID NO: 36, SEQ ID NO: 38, SEQ ID NO: 41, SEQ ID NO: 43, SEQ ID NO: 44, SEQ ID NO: 45, SEQ ID NO: 46, SEQ ID NO: 49, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 62, SEQ ID NO: 69, SEQ ID NO: 72, SEQ ID NO: 73, SEQ ID NO: 77, and/or SEQ ID NO: 78 above the reference level indicates that the patient suffers from lung cancer, and/or
the level of the RNA molecule selected from the group consisting of SEQ ID NO: 4, SEQ ID NO: 10, SEQ ID NO: 14, SEQ ID NO: 16, SEQ ID NO: 17, SEQ ID NO: 21, SEQ ID NO: 22, SEQ ID NO: 25, SEQ ID NO: 26, SEQ ID NO: 28, SEQ ID NO: 29, SEQ ID NO: 30, SEQ ID NO: 31, SEQ ID NO: 32, SEQ ID NO: 35, SEQ ID NO: 39, SEQ ID NO: 40, SEQ ID NO: 42, SEQ ID NO: 47, SEQ ID NO: 48, SEQ ID NO: 52, SEQ ID NO: 61, SEQ ID NO: 63, SEQ ID NO: 64, SEQ ID NO: 65, SEQ ID NO: 66, SEQ ID NO: 67, SEQ ID NO: 68, SEQ ID NO: 70, SEQ ID NO: 71, SEQ ID NO: 74, SEQ ID NO: 75, SEQ ID NO: 76, and/or SEQ ID NO: 79 below the reference level indicates that the patient suffers from lung cancer.

Alternatively or additionally, the reference levels are the levels of the at least two RNA molecules determined empirically by measuring a number of reference blood samples from subjects known to suffer from lung cancer, wherein
the level of the RNA molecule selected from the group consisting of SEQ ID NO: 1, SEQ ID NO: 2, SEQ ID NO: 3, SEQ ID NO: 6, SEQ ID NO: 7, SEQ ID NO: 8, SEQ ID NO: 9, SEQ ID NO: 11, SEQ ID NO: 12, SEQ ID NO: 15, SEQ ID NO: 18, SEQ ID NO: 19, SEQ ID NO: 20, SEQ ID NO: 23, SEQ ID NO: 24, SEQ ID NO: 33, SEQ ID NO: 34, SEQ ID NO: 36, SEQ ID NO: 38, SEQ ID NO: 41, SEQ ID NO: 43, SEQ ID NO: 44, SEQ ID NO: 45, SEQ ID NO: 46, SEQ ID NO: 49, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 62, SEQ ID NO: 69, SEQ ID NO: 72, SEQ ID NO: 73, SEQ ID NO: 77, and/or SEQ ID NO: 78 below the reference level indicates that the patient is healthy (does not suffer from lung cancer), and/or
the level of the RNA molecule selected from the group consisting of SEQ ID NO: 4, SEQ ID NO: 10, SEQ ID NO: 14, SEQ ID NO: 16, SEQ ID NO: 17, SEQ ID NO: 21, SEQ ID NO: 22, SEQ ID NO: 25, SEQ ID NO: 26, SEQ ID NO: 28, SEQ ID NO: 29, SEQ ID NO: 30, SEQ ID NO: 31, SEQ ID NO: 32, SEQ ID NO: 35, SEQ ID NO: 39, SEQ ID NO: 40, SEQ ID NO: 42, SEQ ID NO: 47, SEQ ID NO: 48, SEQ ID NO: 52, SEQ ID NO: 61, SEQ ID NO: 63, SEQ ID NO: 64, SEQ ID NO: 65, SEQ ID NO: 66, SEQ ID NO: 67, SEQ ID NO: 68, SEQ ID NO: 70, SEQ ID NO: 71, SEQ ID NO: 74, SEQ ID NO: 75, SEQ ID NO: 76, and/or SEQ ID NO: 79 above the reference level indicates that the patient is healthy (does not suffer from lung cancer).

Specifically, the level of the RNA molecule is at least 0.6-fold or 0.7-fold, more specifically at least 0.8-fold or 0.9-fold, even more specifically at least 1.2-fold or 1.5-fold, and still even more specifically at least 2.0-fold or 3.0-fold above/below the reference level. For example, the level of the RNA molecule is at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold above/below the reference level.

In alternative or additional embodiment (i), the level of the at least one RNA molecule is used in a mathematical computation to obtain a numerical value (z). In particular, the level of the at least one RNA molecule is used in a mathematical computation to obtain a numerical value (z) by multiplying each level with a corresponding factor (weight), summing all resulting products, and adding a constant (bias). The result of this computation (x) is used as input to a logistic function 1/(1+exp(−x)), resulting in z. The mathematical computation is called logistic regression, where weights and bias are optimized based on the data (RNA levels) to predict the outcome (cancer or healthy).

Preferably, the numerical value (z) is compared to a (an empirically determined) cut-off score (y). The cut-off score (y) allows to classify a patient as a patient suffering from lung cancer or as a patient not suffering from lung cancer (being healthy).

With respect to embodiment (i):
More preferably, the numerical value (z) is calculated for SEQ ID NO: 1 as follows:
z=f(1.00*x1+−5.24), f(x)=1/(1+exp(−x)))), where x1 is the standardized log 2-RPM expression of SEQ ID NO 1, RPM stands for reads per million, the cut-off score (y) is 0.5, and
wherein a numerical value (z) of
>0.5 indicates that the patient suffers from lung cancer, or
<0.5 indicates that the patient is healthy (does not suffer from lung cancer).

In alternative or additional embodiments (ii) and/or (iii), the levels of the at least two RNA molecules are used in a mathematical computation to obtain a numerical value (z). In particular, the levels of the at least two RNA molecules are used in a mathematical computation to obtain a numerical value (z) by multiplying each level with a corresponding factor (weight), summing all resulting products, and adding a constant (bias). The result of this computation (x) is used as input to a logistic function 1/(1+exp(−x)), resulting in z. The mathematical computation is called logistic regression, where weights and bias are optimized based on the data (RNA levels) to predict the outcome (cancer or healthy).

Specifically, the numerical value is obtained by summarizing the weighted levels of the at least two RNA molecules, e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 RNA molecules (such as 71 or 79 RNA molecules). In this regard, the method further comprises the step of determining a numerical value by summarizing the weighted levels of the at least two RNA molecules, e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 RNA molecules (such as 71 or 79 RNA molecules).

With the at least two RNA molecules,
the RNA molecules having a nucleotide sequence according to SEQ ID NO: 1 and SEQ ID NO: 2 and optionally at least one RNA molecule having a nucleotide sequence according to SEQ ID NO: 3 to SEQ ID NO: 71, or
the RNA molecules having a nucleotide sequence according to SEQ ID NO: 1 and SEQ ID NO: 2 and optionally at least one RNA molecule having a nucleotide sequence according to SEQ ID NO: 3 to SEQ ID NO: 79 are meant.

Alternatively, with the at least two RNA molecules, the RNA molecules having a nucleotide sequence according to SEQ ID NO: 2 and SEQ ID NO: 3 and optionally at least one RNA molecule having a nucleotide sequence according to SEQ ID NO: 1, SEQ ID NO: 4 to SEQ ID NO: 79 are meant.

The at least two RNA molecules can also be designated as RNA molecule signature.

Preferably, the numerical value is compared to a (an empirically determined) cut-off score (y). The cut-off score (y) allows to classify a patient as a patient suffering from lung cancer or as a patient not suffering from lung cancer (being healthy).

With respect to embodiment (ii):
More preferably, the numerical value (z) is calculated for the signature: SEQ ID NO: 1 and SEQ ID NO: 2 as follows:

$$z = f ( 0.9 {5}^{*} x 1 + 0.71 * x 2 + - 5.71 ) , f ( x ) = 1 / ( 1 + exp ( - x ) ) ) ) ,$$

wherein x1 and x2 is the standardized log 2-RPM expression of SEQ ID NO: 1 and SEQ ID NO: 2, RPM stands for reads per million, the cut-off score (y) is 0.5, and
wherein a numerical value (z) of
>0.5 indicates that the patient suffers from lung cancer, or
<0.5 indicates that the patient is healthy (does not suffer from lung cancer).

Even more preferably, the numerical value (z) is calculated for the signature: SEQ ID NO: 1, SEQ ID NO: 2, SEQ ID NO: 19, SEQ ID NO: 35, SEQ ID NO: 39, and SEQ ID NO: 48 as follows:

$$z = f ( 0.94 * x 1 + 0.86 * x 2 + - 0.07 * x 19 + - 0.33 * x 35 + - 0.15 * x 39 + - 0.33 * x 48 + - 3.86 ) ,$$

$$f ( x ) = 1 / ( 1 + exp ( - x ) ) ) ) ,$$

wherein x1, x2, x19, x35, x39, and x48 is the standardized log 2-RPM expression of SEQ ID NO: 1, SEQ ID NO: 2, SEQ ID NO: 19, SEQ ID NO: 35, SEQ ID NO: 39, and SEQ ID NO: 48, respectively, RPM stands for reads per million, the cut-off score (y) is 0.5, and
wherein a numerical value (z) of
>0.5 indicates that the patient suffers from lung cancer, or
<0.5 indicates that the patient is healthy (does not suffer from lung cancer).

Alternatively, the numerical value (z) is calculated for the signature: SEQ ID NO: 1, SEQ ID NO: 2, SEQ ID NO: 23, SEQ ID NO: 39, and SEQ ID NO: 48 as follows:

$$y = f ( 0.44 * x 1 + 0.83 * x 2 + 0.45 * x 23 + - 0.07 * x 39 + - 0.25 * x 48 + - 4.34 ) ,$$

$$f ( x ) = 1 / ( 1 + exp ( - x ) ) ) ) ,$$

wherein x1, x2, x23, x39, and x48 is the standardized log 2-RPM expression of SEQ ID NO: 1, SEQ ID NO: 2, SEQ ID NO: 23, SEQ ID NO: 39, and SEQ ID NO: 48, respectively, RPM stands for reads per million, the cut-off score (y) is 0.5, and
wherein a numerical value (z) of
>0.5 indicates that the patient suffers from lung cancer, or
<0.5 indicates that the patient is healthy (does not suffer from lung cancer).

Alternatively, the numerical value (z) is calculated for the signature: SEQ ID NO: 1, SEQ ID NO:
2, SEQ ID NO: 6, SEQ ID NO: 7, and SEQ ID NO: 9 as follows:

$$z = f ( 0.84 * x 1 + 0.6 * x 2 + 0.85 * x 6 + 0.23 * x 7 + 0.3 * x 9 + - 6.44 ) ,$$

$$f ( x ) = 1 / ( 1 + exp ( - x ) ) ) ) ,$$

wherein x1, x2, x6, x7, and x9 is the standardized log 2-RPM expression of SEQ ID NO: 1, SEQ ID NO: 2, SEQ ID NO: 6, SEQ ID NO: 7, SEQ ID NO: 9, respectively, RPM stands for reads per million, the cut-off score (y) is 0.5, and

wherein a numerical value (z) of
>0.5 indicates that the patient suffers from lung cancer, or
<0.5 indicates that the patient is healthy (does not suffer from lung cancer).

Alternatively, the numerical value (z) is calculated for the signature: SEQ ID NO: 1, SEQ ID NO: 2, SEQ ID NO: 6, SEQ ID NO: 7, and SEQ ID NO: 11, and SEQ ID NO: 18 as follows:

$$y = f ( 0.19 * x 1 + 0.62 * x 2 + 0.87 * x 6 + 0.32 * x 7 + 0.4 * x 11 + 0.65 * x 18 + - 6.12 ) ,$$

$$f ( x ) = 1 / ( 1 + exp ( - x ) ) ) ) ,$$

wherein x1, x2, x6, x7, x11, and x18 is the standardized log 2-RPM expression of SEQ ID NO: 1, SEQ ID NO: 2, SEQ ID NO: 6, SEQ ID NO: 7, SEQ ID NO: 11, and SEQ ID NO: 18, respectively, RPM stands for reads per million, the cut-off score (y) is 0.5, and
wherein a numerical value (z) of
>0.5 indicates that the patient suffers from lung cancer, or
<0.5 indicates that the patient is healthy (does not suffer from lung cancer).

Still even more preferably, the numerical value (z) is calculated for the signature: SEQ ID NO: 1, SEQ ID NO: 2, SEQ ID NO: 19, SEQ ID NO: 35, SEQ ID NO: 38, SEQ ID NO: 39, SEQ ID NO: 40, SEQ ID NO: 43, SEQ ID NO: 48, and SEQ ID NO: 49 as follows:

$$z = f ( 0.93 * x 1 + 0.83 * x 2 + - 0.09 * x 19 + - 0.33 * x 35 + - 0.11 * x 38 + - 0.13 * x 39 + - 0.12 * x 40 + 0.17 * x 43 + - 0.29 * x 48 + 0.1 * x 49 + - 4.41 ) ,$$

$$f ( x ) = 1 / ( 1 + exp ( - x ) ) ) ) ,$$

wherein x1, x2, x19, x35, x38, x39, x40, x43, x48, and x49 is the standardized log 2-RPM expression of SEQ ID NO: 1, SEQ ID NO: 2, SEQ ID NO: 19, SEQ ID NO: 35, SEQ ID NO: 38, SEQ ID NO: 39, SEQ ID NO: 40, SEQ ID NO: 43, SEQ ID NO: 48, and SEQ ID NO: 49, respectively, RPM stands for reads per million, the cut-off score (y) is 0.5, and
wherein a numerical value (z) of
>0.5 indicates that the patient suffers from lung cancer, or
<0.5 indicates that the patient is healthy (does not suffer from lung cancer).

Alternatively, the numerical value (z) is calculated for the signature: SEQ ID NO: 1, SEQ ID NO: 2, SEQ ID NO: 17, SEQ ID NO: 23, SEQ ID NO: 35, SEQ ID NO: 39, SEQ ID NO: 45, SEQ ID NO: 48, SEQ ID NO: 52, and SEQ ID NO: 53 as follows:

$$z = f ( 0.52 * x 1 + 0.93 * x 2 + - 0.22 * x 17 + 0.43 * x 23 + - 0.22 * x 35 + - 0.08 * x 39 + - 0.06 * x 45 + - 0.22 * x 48 + - 0.32 * x 52 + 0.02 * x 53 + - 1.32 ) ,$$

$$f ( x ) = 1 / ( 1 + exp ( - x ) ) ) ) ,$$

wherein x1, x2, x17, x23, x35, x39, x45, x48, x52, and x53 is the standardized log 2-RPM expression of SEQ ID NO: 1, SEQ ID NO: 2, SEQ ID NO: 17, SEQ ID NO: 23, SEQ ID NO: 35, SEQ ID NO: 39, SEQ ID NO: 45, SEQ ID NO: 48, SEQ ID NO: 52, and SEQ ID NO: 53, respectively, RPM stands for reads per million, the cut-off score (y) is 0.5, and
wherein a numerical value (z) of
>0.5 indicates that the patient suffers from lung cancer, or
<0.5 indicates that the patient is healthy (does not suffer from lung cancer).

Alternatively, the numerical value (z) is calculated for the signature: SEQ ID NO: 1, SEQ ID NO: 2, SEQ ID NO: 6, SEQ ID NO: 7, SEQ ID NO: 9, SEQ ID NO: 19, SEQ ID NO: 25, SEQ ID NO: 29, SEQ ID NO: 30, and SEQ ID NO: 38 as follows:

$$z = f ( 0.83 * x 1 + 0.77 * x 2 + 0.69 * x 6 + 0.31 * x 7 + 0.34 * x 9 + - 0.03 * x 19 + - 0.58 * x 25 + - 0.33 * x 29 + - 0.28 * x 30 + - 0.19 * x 38 + 3.17 ) ,$$

$$f ( x ) = 1 / ( 1 + exp ( - x ) ) ) ) ,$$

wherein x1, x2, x6, x7, x9, x19, x25, x29, x30, and x38 is the standardized log 2-RPM expression of SEQ ID NO: 1, SEQ ID NO: 2, SEQ ID NO: 6, SEQ ID NO: 7, SEQ ID NO: 9, SEQ ID NO: 19, SEQ ID NO: 25, SEQ ID NO: 29, SEQ ID NO: 30, and SEQ ID NO: 38, respectively, RPM stands for reads per million, the cut-off score (y) is 0.5, and
wherein a numerical value (z) of
>0.5 indicates that the patient suffers from lung cancer, or
<0.5 indicates that the patient is healthy (does not suffer from lung cancer).

Alternatively, the numerical value (z) is calculated for the signature: SEQ ID NO: 1, SEQ ID NO: 2, SEQ ID NO: 3, SEQ ID NO: 6, SEQ ID NO: 7, SEQ ID NO: 11, SEQ ID NO: 12, SEQ ID NO: 14, SEQ ID NO: 18, and SEQ ID NO: 28 as follows:

$$z = f ( 0.24 * x 1 + 0.74 * x 2 + 0.19 * x 3 + 0.68 * x 6 + 0.36 * x 7 + 0.26 * x 11 + 0.26 * x 12 + - 0 \text{.42} * x 14 + 0.66 * x 18 + - 0.94 * x 28 + 4.76 ) ,$$

$$f ( x ) = 1 / ( 1 + exp ( - x ) ) ) ) ,$$

wherein x1, x2, x3, x6, x7, x11, x12, x14, x18, and x28 is the standardized log 2-RPM expression of SEQ ID NO: 1, SEQ ID NO: 2, SEQ ID NO: 3, SEQ ID NO: 6, SEQ ID NO: 7, SEQ ID NO: 11, SEQ ID NO: 12, SEQ ID NO: 14, SEQ ID NO: 18, and SEQ ID NO: 28, respectively, RPM stands for reads per million, the cut-off score (y) is 0.5, and
wherein a numerical value (z) of
>0.5 indicates that the patient suffers from lung cancer, or
<0.5 indicates that the patient is healthy (does not suffer from lung cancer).

Most preferably, the numerical value (z) is calculated for the (54) signature: SEQ ID NO: 1 to SEQ ID NO: 54 as follows: z=f(0.45*x1+0.83*x2+0.74*x3+0.50*x4+−0.48*x5+0.74*x6+0.48*x7+0.55*x8+0.64*x9+−0.72*x10+0.39*x11+0.25*x12+0.35*x13+−0.34*x14+−0.43*x15+−0.40*x16+−0.34*x17+0.14*x18+−0.32*x19+−0.52*x20+−0.28*x21+−0.32*x22+−0.28*x23+0.20*x24+−0.33*x25+−0.24*x26+0.26*x27+0.44*x28+−0.11*x29+−0.12*x30+−0.10*x31+0.25*x32+−0.26*x33+0.16*x34+−0.15*x35+−0.24*x36+0.10*x37+−0.11*x38+−0.08*x39+0.08*x40+0.05*x41+−0.10*x42+0.06*x43+0.06*x44+0.12*x45+−0.06*x46+0.10*x47+−0.02*x48+0.01*x49+−0.04*x50+0.02*x51+0.01*x52+0.04*x53+0.05*x54+0.21), f(x)=1/(1+exp(−x)))), wherein x1 to x54 is the standardized log 2-RPM expression of SEQ ID NO: 1 to SEQ ID NO: 54, respectively, RPM stands for reads per million, the cut-off score (y) is 0.5, and
wherein a numerical value (z) of
>0.5 indicates that the patient suffers from lung cancer, or
<0.5 indicates that the patient is healthy (does not suffer from lung cancer).

Alternatively, the numerical value (z) is calculated for the (38) signature: SEQ ID NO: 1, SEQ ID NO: 2, SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, and SEQ ID NO: 71 as follows:

$$z = f ( 0.05 * x 1 + 0.83 * x 2 + 0.27 * x 3 + 0.89 * x 6 + 0.35 * x 7 + 0.29 * x 8 + - 0.56 * x 10 + 0.14 * x 11 + 0.13 * x 12 + - 0.16 * x 14 + - 0.21 * x 16 + - 0.29 * x 17 + 0.37 * x 18 + - 0.21 * x 21 + - 0.26 * x 22 + - 0.15 * x 24 + - 0.26 * x 26 + - 0.74 * x 28 + - 0.17 * x 29 + - 0.17 * x 31 + 0.17 * x 34 + 0.16 * x 55 + 0.18 * x 56 + 0.19 * x 57 + 0.08 * x 58 + 0.27 * x 59 + 0.39 * x 60 + 0.5 * x 61 + 0.24 * x 62 + - 0.1 * x 63 + - 0.19 * x 64 + - 0.12 * x 65 + - 0.54 * x 66 + - 0.07 * x 67 + - 0.2 * x 68 + - 0.25 * x 69 + 0.81 * x 70 + - 0.19 * x 71 + 0.33 ) ,$$

$$f ( x ) = 1 / ( 1 + exp ( - x ) ) ) ) ,$$

wherein x1, x2, x3, x6, x7, x8, x10, x11, x12, x14, x16, x17, x18, x21, x22, x24, x26, x28, x29, x31, x34, x55 to x71 is the standardized log 2-RPM expression of SEQ ID NO: 1, SEQ ID NO: 2, SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, and SEQ ID NO: 71, respectively, RPM stands for reads per million, the cut-off score (y) is 0.5, and
wherein a numerical value (z) of
>0.5 indicates that the patient suffers from lung cancer, or
<0.5 indicates that the patient is healthy (does not suffer from lung cancer).

With respect to embodiment (iii):
More preferably, the numerical value (z) is calculated for the (2) signature: SEQ ID NO: 2 and SEQ ID NO: 3 as follows:

$$z = f ( 0.800 * x 2 + 1.342 * x 3 + - 8.733 ) ,$$

$$f ( x ) = 1 / ( 1 + exp ( - x ) ) ) ) ,$$

wherein x2, x3 is the log 2-RPM expression of SEQ ID NO: 2, SEQ ID NO: 3, respectively, RPM stands for reads per million, the cut-off score (y) is 0.5, and
wherein a numerical value (z) of
>0.5 indicates that the patient suffers from lung cancer, or
<0.5 indicates that the patient is healthy (does not suffer from lung cancer).

Even more preferably, the numerical value (z) is calculated for the (18) signature: SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, and SEQ ID NO: 79 as follows:

$$z = f ( 0.792 * x 2 + 0.54 * x 3 + 0.521 * x 7 + 0.26 * x 11 + - 0.284 * x 14 + - 0.306 * x 17 + 0.399 * x 18 + - 0.429 * x 21 + - 0.693 * x 28 + 0.295 * x 56 + 0.452 * x 72 + 0.38 * x 73 + - 0.3 * x 74 + - 0.297 * x 75 + - 0.219 * x 76 + 0.157 * x 77 + 0.0658 * x 78 + - 0.0421 * x 79 + 5.203 ) ,$$

$$f ( x ) = 1 / ( 1 + exp ( - x ) ) ) ) ,$$

wherein x2, x3, x7, x11, x14, x17, x18, x21, x28, x56, x72, x73, x74, x75, x76, x77, x78, x79 is the log 2-RPM expression of SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, respectively, RPM stands for reads per million, the cut-off score (y) is 0.5, and
wherein a numerical value (z) of
>0.5 indicates that the patient suffers from lung cancer, or
<0.5 indicates that the patient is healthy (does not suffer from lung cancer).

Even more preferably, the numerical value (z) is calculated for the (6) signature (Mitra samples):
SEQ ID NO: 2, SEQ ID NO: 3, SEQ ID NO: 7, SEQ ID NO: 17, SEQ ID NO: 18, and SEQ ID NO: 75 as follows:

$$z = f ( 19.621 * x 2 + 0.638 * x 3 + 1.171 * x 7 + - 0.492 * x 17 + - 2.4 * x 18 + 1.049 * x 75 + 3.185 ) ,$$

$$f ( x ) = 1 / ( 1 + exp ( - x ) ) ) ) ,$$

wherein x2, x3, x7, x17, x18, x75 is the log 2-RPM expression of SEQ ID NO: 2, SEQ ID NO: 3, SEQ ID NO: 7, SEQ ID NO: 17, SEQ ID NO: 18, SEQ ID NO: 75, respectively, RPM stands for reads per million, the cut-off score (y) is 0.5, and
wherein a numerical value (z) of
>0.5 indicates that the patient suffers from lung cancer, or
<0.5 indicates that the patient is healthy (does not suffer from lung cancer).

In a second aspect, the present invention relates to a (an in vitro) method of monitoring a patient suspected of having lung cancer or a patient suffering from lung cancer comprising the step of:
(i) determining the level of at least one RNA molecule in a blood sample from a patient, wherein the at least one RNA molecule comprises/has a nucleotide sequence according to SEQ ID NO: 1, is a fragment thereof, or has a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto,
(ii) determining the levels of at least two RNA molecules in a blood sample from a patient, wherein the at least two RNA molecules comprise a first RNA molecule and a second RNA molecule,
   wherein the first RNA molecule comprises/has a nucleotide sequence according to SEQ ID NO: 1, is a fragment thereof, or has a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and the second RNA molecule comprises/has a nucleotide sequence according to SEQ ID NO: 2, is a fragment thereof, or has a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto, or
(iii) determining the levels of at least two RNA molecules in a blood sample from a patient, wherein the at least two RNA molecules comprise a first RNA molecule and a second RNA molecule,
   wherein the first RNA molecule comprises/has a nucleotide sequence according to SEQ ID NO: 2, is a fragment thereof, or has a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and the second RNA molecule comprises/has a nucleotide sequence according to SEQ ID NO: 3, is a fragment thereof, or has a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Preferably,
the first RNA molecule comprises a nucleotide sequence according to SEQ ID NO: 1 and the second RNA molecule comprises a nucleotide sequence according to SEQ ID NO: 2, or the first RNA molecule comprises a nucleotide sequence according to SEQ ID NO: 2 and the second RNA molecule comprises a nucleotide sequence according to SEQ ID NO: 3.

Especially, the at least two RNA molecules are comprised in a set. In other words, the at least two RNA molecules represent a signature. The signature can encompass, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 RNA molecules, specifically 2, 5, 6, 10, 18, 38, or 54 RNA molecules (RNA molecule signature).

In embodiment (i),
the level of at least one further RNA molecule selected from the group consisting of an RNA molecule comprising/having a nucleotide sequence according to SEQ ID NO: 2 to SEQ ID NO: 71, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto is determined, or
the level of at least one further RNA molecule selected from the group consisting of an RNA molecule comprising/having a nucleotide sequence according to SEQ ID NO: 2 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto is determined.

Thus, the level of the RNA molecule comprising a nucleotide sequence according to SEQ ID NO: 1, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and
the level of at least one further RNA molecule selected from the group consisting of an RNA molecule comprising a nucleotide sequence according to SEQ ID NO: 2 to SEQ ID NO: 71, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are determined in the blood sample from the patient, or
the level of at least one further RNA molecule selected from the group consisting of an RNA molecule comprising a nucleotide sequence according to SEQ ID NO: 2 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are determined in the blood sample from the patient.

Specifically, the level of the RNA molecule comprising
(i) a nucleotide sequence according to SEQ ID NO: 1 to SEQ ID NO: 71 or SEQ ID NO: 1 to SEQ ID NO: 79,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iii) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii)
is determined in the blood sample.

In embodiment (ii),
the level of at least one further RNA molecule selected from the group consisting of an RNA molecule comprising/having a nucleotide sequence according to SEQ ID NO: 3 to SEQ ID NO: 71, a fragment thereof, and a sequence having at least 80% sequence identity thereto is determined, or
the level of at least one further RNA molecule selected from the group consisting of an RNA molecule comprising/having a nucleotide sequence according to SEQ ID NO: 3 to SEQ ID NO: 79, a fragment thereof, and a sequence having at least 80% sequence identity thereto is determined. Thus, the level of the RNA molecule comprising a nucleotide sequence according to SEQ ID NO: 1, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto as well as the level of the RNA molecule comprising a nucleotide sequence according to SEQ ID NO: 2, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and
the level of at least one further RNA molecule selected from the group consisting of an RNA molecule comprising a nucleotide sequence according to SEQ ID NO: 3 to SEQ ID NO: 71, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are determined in the blood sample from the patient, or
the level of at least one further RNA molecule selected from the group consisting of an RNA molecule comprising a nucleotide sequence according to SEQ ID NO: 3 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are determined in the blood sample from the patient.

Specifically, the level of the RNA molecule comprising
(i) a nucleotide sequence according to SEQ ID NO: 1 to SEQ ID NO: 71 or SEQ ID NO: 1 to SEQ ID NO: 79,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iii) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii)
is determined in the blood sample.

Preferably,
(i) the level of at least one further RNA molecule selected from the group consisting of an RNA molecule comprising a nucleotide sequence according to SEQ ID NO: 19, SEQ ID NO: 35, SEQ ID NO: 39, SEQ ID NO: 48, a fragment thereof, and a sequence having at least 80% sequence identity thereto is determined,
(ii) the level of at least one further RNA molecule selected from the group consisting of an RNA molecule comprising a nucleotide sequence according to SEQ ID NO: 23, SEQ ID NO: 39, SEQ ID NO: 48, a fragment thereof, and a sequence having at least 80% sequence identity thereto is determined,
(iii) the level of at least one further RNA molecule selected from the group consisting of an RNA molecule comprising a nucleotide sequence according to SEQ ID NO: 6, SEQ ID NO: 7, SEQ ID NO: 9, a fragment thereof, and a sequence having at least 80% sequence identity thereto is determined,
(iv) the level of at least one further RNA molecule selected from the group consisting of an RNA molecule comprising a nucleotide sequence according to SEQ ID NO: 6, SEQ ID NO: 7, SEQ ID NO: 11, SEQ ID NO: 18, a fragment thereof, and a sequence having at least 80% sequence identity thereto is determined,
(v) the level of at least one further RNA molecule selected from the group consisting of an RNA molecule comprising a nucleotide sequence according to SEQ ID NO: 19, SEQ ID NO: 35, SEQ ID NO: 38, SEQ ID NO: 39, SEQ ID NO: 40, SEQ ID NO: 43, SEQ ID NO: 48, SEQ ID NO: 49, a fragment thereof, and a sequence having at least 80% sequence identity thereto is determined,
(vi) the level of at least one further RNA molecule selected from the group consisting of an RNA molecule comprising a nucleotide sequence according to SEQ ID NO: 17, SEQ ID NO: 23, SEQ ID NO: 35, SEQ ID NO: 39, SEQ ID NO: 45, SEQ ID NO: 48, SEQ ID NO: 52, SEQ ID NO: 53, a fragment thereof, and a sequence having at least 80% sequence identity thereto is determined,
(vii) the level of at least one further RNA molecule selected from the group consisting of an RNA molecule comprising a nucleotide sequence according to SEQ ID NO: 6, SEQ ID NO: 7, SEQ ID NO: 9, SEQ ID NO: 19, SEQ ID NO: 25, SEQ ID NO: 29, SEQ ID NO: 30, SEQ ID NO: 38, a fragment thereof, and a sequence having at least 80% sequence identity thereto is determined,
(viii) the level of at least one further RNA molecule selected from the group consisting of an RNA molecule comprising a nucleotide sequence according to SEQ ID NO: 3, SEQ ID NO: 6, SEQ ID NO: 7, SEQ ID NO: 11, SEQ ID NO: 12, SEQ ID NO: 14, SEQ ID NO: 18, SEQ ID NO: 28, a fragment thereof, and a sequence having at least 80% sequence identity thereto is determined,
(ix) the level of at least one further RNA molecule selected from the group consisting of an RNA molecule comprising a nucleotide sequence according to SEQ ID NO: 3 to SEQ ID NO: 54, a fragment thereof, and a sequence having at least 80% sequence identity thereto is determined, or
(x) the level of at least one further RNA molecule selected from the group consisting of an RNA molecule comprising a nucleotide sequence according to SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, a fragment thereof, and a sequence having at least 80% sequence identity thereto is determined.

More preferably, the levels of the RNA molecules comprising a nucleotide sequence according to
(i) SEQ ID NO: 1, SEQ ID NO: 2, SEQ ID NO: 19, SEQ ID NO: 35, SEQ ID NO: 39, and SEQ ID NO: 48,
(ii) SEQ ID NO: 1, SEQ ID NO: 2, SEQ ID NO: 23, SEQ ID NO: 39, and SEQ ID NO: 48,
(iii) SEQ ID NO: 1, SEQ ID NO: 2, SEQ ID NO: 6, SEQ ID NO: 7, and SEQ ID NO: 9,
(iv) SEQ ID NO: 1, SEQ ID NO: 2, SEQ ID NO: 6, SEQ ID NO: 7, SEQ ID NO: 11, and SEQ ID NO: 18,
(v) SEQ ID NO: 1, SEQ ID NO: 2, SEQ ID NO: 19, SEQ ID NO: 35, SEQ ID NO: 38, SEQ ID NO: 39, SEQ ID NO: 40, SEQ ID NO: 43, SEQ ID NO: 48, and SEQ ID NO: 49,
(vi) SEQ ID NO: 1, SEQ ID NO: 2, SEQ ID NO: 17, SEQ ID NO: 23, SEQ ID NO: 35, SEQ ID NO: 39, SEQ ID NO: 45, SEQ ID NO: 48, SEQ ID NO: 52, and SEQ ID NO: 53,
(vii) SEQ ID NO: 1, SEQ ID NO: 2, SEQ ID NO: 6, SEQ ID NO: 7, SEQ ID NO: 9, SEQ ID NO: 19, SEQ ID NO: 25, SEQ ID NO: 29, SEQ ID NO: 30, and SEQ ID NO: 38,
(viii) SEQ ID NO: 1, SEQ ID NO: 2, SEQ ID NO: 3, SEQ ID NO: 6, SEQ ID NO: 7, SEQ ID NO: 11, SEQ ID NO: 12, SEQ ID NO: 14, SEQ ID NO: 18, and SEQ ID NO: 28,
(ix) SEQ ID NO: 1 to SEQ ID NO: 54, or
(x) SEQ ID NO: 1, SEQ ID NO: 2, SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, and SEQ ID NO: 71
are determined.

In embodiment (iii), the level of at least one further RNA molecule selected from the group consisting of an RNA molecule comprising/having a nucleotide sequence according to SEQ ID NO: 1, SEQ ID NO: 4 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto is determined.

Thus, the level of the RNA molecule comprising a nucleotide sequence according to SEQ ID NO: 2, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto as well as the level of the RNA molecule comprising a nucleotide sequence according to SEQ ID NO: 3, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and the level of at least one further RNA molecule selected from the group consisting of an RNA molecule comprising a nucleotide sequence according to SEQ ID NO: 1, SEQ ID NO: 4 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are determined in the blood sample from the patient.

Specifically, the level of the RNA molecule comprising
(i) a nucleotide sequence according to SEQ ID NO: 1 to SEQ ID NO: 79,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iii) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii) is determined in the blood sample.

Preferably,
(i) the level of at least one further RNA molecule selected from the group consisting of an RNA molecule comprising a nucleotide sequence according to SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72 to SEQ ID NO: 79, a fragment thereof, and a sequence having at least 80% sequence identity thereto is determined, or
(ii) the level of at least one further RNA molecule selected from the group consisting of an RNA molecule comprising a nucleotide sequence according to SEQ ID NO: 7, SEQ ID NO: 17, SEQ ID NO: 18, SEQ ID NO: 75, a fragment thereof, and a sequence having at least 80% sequence identity thereto is determined.

More preferably, the levels of the RNA molecules comprising a nucleotide sequence according to
(i) SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, and SEQ ID NO: 79, or
(ii) SEQ ID NO: 2, SEQ ID NO: 3, SEQ ID NO: 7, SEQ ID NO: 17, SEQ ID NO: 18, and SEQ ID NO: 75 are determined.

In preferred embodiment (i), the level of the at least one RNA molecule is compared to a reference. Particularly, the level of the at least one RNA molecule is compared to a reference level of said RNA molecule.

The above comparison allows to monitor/determine the course of lung cancer in a patient suffering from lung cancer. It may be determined that the lung cancer worsens in the patient, that the lung cancer does not worsen/is stable in the patient, or that the lung cancer improves in the patient. The above comparison also allows to monitor/determine whether lung cancer has developed in the patient.

The reference, such as the reference level, may be any reference, such as reference level, which allows to monitor or detect lung cancer in a patient. It may be obtained from (control) subjects (i.e. subjects different from the patient to be tested such as subjects having lung cancer/known to have lung cancer or healthy subjects).

More particularly, the reference level is the level of the at least one RNA molecule determined empirically by measuring a number of reference blood samples from subjects known to be healthy (not suffering from lung cancer), and/or from subjects known to suffer from lung cancer.

For example,
the reference level is determined from at least 2, at least 10, at least 50, at least 100, at least 200, at least 300, at least 400, at least 500, at least 1000, at least 1500, at least 2000 at least 5000 reference samples from healthy subjects, and/or
the reference level is determined from at least 2, at least 10, at least 50, at least 100, at least 200, at least 300, at least 400, at least 500, at least 1000, at least 1500, at least 2000 at least 5000 reference samples from subjects known to suffer from lung cancer.

Specifically, the reference level is an average reference level. It is determined by measuring reference levels of control subjects (e.g. healthy subjects or subjects having lung cancer/known to have lung cancer) and calculating the “average” value (e.g. mean, median or modal value) thereof. It is preferred that the reference blood sample is from the same source (e.g. blood cells, serum, or plasma) than the blood sample isolated from the patient to be tested. It is further preferred that the reference level is obtained from control subjects (e.g. healthy subjects or subjects having lung cancer/known to have lung cancer) of the same gender (e.g. female or male) and/or of a similar age/phase of life (e.g. adults or elderly) than the patient to be tested. Especially, the reference level represents an average value of the RNA molecule in a healthy population and/or the reference level represents an average value of the RNA molecule in a population of subjects having lung cancer/known to have lung cancer.

In preferred embodiments (ii) and/or (iii), the levels of the at least two RNA molecules are compared to references. Particularly, the levels of the at least two RNA molecules are compared to reference levels of said RNA molecules.

The above comparison allows to diagnose lung cancer in a patient, in particular in a patient suspected of suffering from lung cancer. The patient may be diagnosed as suffering from lung cancer, i.e. being diseased, or as not suffering from lung cancer, i.e. being healthy.

The references, such as the reference levels, may be references, such as reference levels, which allow to determine whether the patient suffers from lung cancer or not. The reference levels may be obtained from (control) subjects (i.e. subjects different from the patient to be tested such as healthy subjects and/or subjects known to have lung cancer) or from the same patient. In the latter case, the patient may be retested for lung cancer, e.g. in the form of a longitudinal monitoring. It may be determined that the patient is now affected by lung cancer or still not affected by lung cancer.

More particularly, the reference levels are the levels of the at least two RNA molecules determined empirically by measuring a number of reference blood samples from subjects known to be healthy (not suffering from lung cancer), and/or from subjects known to suffer from lung cancer.

For example,
the reference levels are determined from at least 2, at least 10, at least 50, at least 100, at least 200, at least 300, at least 400, at least 500, at least 1000, at least 1500, at least 2000 at least 5000 reference samples from healthy subjects, and/or
the reference levels are determined from at least 2, at least 10, at least 50, at least 100, at least 200, at least 300, at least 400, at least 500, at least 1000, at least 1500, at least 2000 at least 5000 reference samples from subjects known to suffer from lung cancer.

Specifically, the reference levels are average reference levels. They are determined by measuring reference levels of control subjects (e.g. healthy subjects or subjects having lung cancer/known to have lung cancer) and calculating “average” values (e.g. mean, median or modal value) thereof. Especially, the reference levels represent average values of the RNA molecules in a healthy population and/or the reference levels represent average values of the RNA molecules in a population of subjects having lung cancer/known to have lung cancer. In this respect, it should be noted that for each RNA molecule, a level is determined which is then compared to the respective reference level of the same RNA molecule.

As to the diagnosis/detection/determination of lung cancer in a patient and the respective reference levels (of (control) subjects known to not suffer from lung cancer (i.e. being healthy) and/or (control) subjects known to suffer from lung cancer), it is referred to the first aspect of the present invention.

As already mentioned above, the reference level may be any level which allows to monitor or detect lung cancer in a patient. It may be obtained from (control) subjects (i.e. subjects different from the patient to be tested such as subjects known to have lung cancer or healthy subjects). It may also be obtained from the same individual. Alternatively, the reference levels may be levels which allow to monitor or detect lung cancer in a patient. They may be obtained from (control) subjects (i.e. subjects different from the patient to be tested such as subjects known to have lung cancer or healthy subjects). They may also be obtained from the same individual.

Accordingly, in alternative or additional embodiment (i), said monitoring comprises determining the level of the at least one RNA molecule in a blood sample obtained from a patient at a first point in time and in at least one further blood sample obtained from the (same) patient at a later point in time and comparing said levels determined at the different time points.

This proceeding allows to monitor lung cancer in a patient (being healthy or suffering from lung cancer) over an extended period of time, such as months or years, e.g. 1, 2, 3, 4, 5, 6, 7, 8, 9, 10, or 11 month(s), 1, 2, 3, 4, or 5 year(s).

The time period between the first point in time and the later point(s) in time preferably amounts to at least 1 day, at least 2 days, at least 3 days, at least 4 days, at least 5 days, at least 6 days, at least 7 days (1 week), at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months (1 year), at least 24 months (2 years), at least 3 years, at least 4 years, at least 5 years, at least 6 years, at least 7 years, at least 8 years, at least 9 years, or at least 10 years. For example, the individual may be routinely checked, e.g. once or twice a year. The patient may be (re) tested at 2, 3, 4, 5, 6 7, 8, 9, or 10 time points (first point in time and further point(s) in time).

In alternative or additional embodiments (ii) and/or (iii), said monitoring comprises determining the levels of the at least two RNA molecules in a blood sample obtained from a patient at a first point in time and in at least one further blood sample obtained from the (same) patient at a later point in time and comparing said levels determined at the different time points.

In this respect, it should be noted that for each RNA molecule, a level is determined at a first point in time and at a later point in time which are then further compared with each other.

This proceeding allows to monitor lung cancer in a patient (being healthy or suffering from lung cancer) over an extended period of time, such as months or years, e.g. 1, 2, 3, 4, 5, 6, 7, 8, 9, 10, or 11 month(s), 1, 2, 3, 4, or 5 year(s).

The time period between the first point in time and the later point(s) in time preferably amounts to at least 1 day, at least 2 days, at least 3 days, at least 4 days, at least 5 days, at least 6 days, at least 7 days (1 week), at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months (1 year), at least 24 months (2 years), at least 3 years, at least 4 years, at least 5 years, at least 6 years, at least 7 years, at least 8 years, at least 9 years, or at least 10 years. For example, the individual may be routinely checked, e.g. once or twice a year. The patient may be (re) tested at 2, 3, 4, 5, 6 7, 8, 9, or 10 time points (first point in time and further point(s) in time).

In preferred embodiments (i), (ii), and/or (iii), the level of the RNA molecule selected from the group consisting of SEQ ID NO: 1, SEQ ID NO: 2, SEQ ID NO: 3, SEQ ID NO: 6, SEQ ID NO: 7, SEQ ID NO: 8, SEQ ID NO: 9, SEQ ID NO: 11, SEQ ID NO: 12, SEQ ID NO: 15, SEQ ID NO: 18, SEQ ID NO: 19, SEQ ID NO: 20, SEQ ID NO: 23, SEQ ID NO: 24, SEQ ID NO: 33, SEQ ID NO: 34, SEQ ID NO: 36, SEQ ID NO: 38, SEQ ID NO: 41, SEQ ID NO: 43, SEQ ID NO: 44, SEQ ID NO: 45, SEQ ID NO: 46, SEQ ID NO: 49, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 62, SEQ ID NO: 69, SEQ ID NO: 72, SEQ ID NO: 73, SEQ ID NO: 77, and/or SEQ ID NO: 78 which
(i) increases over time indicates that the patient has developed lung cancer or that lung cancer is worsening in the patient,
(ii) does not change over time indicates that the patient is stable or that lung cancer is not progressing in the patient, or
(iii) decreases over time indicates that lung cancer is improving in the patient.

Specifically,
(i) the patient was healthy at the first point in time and the level which increases over time indicates that the patient has developed lung cancer,
(ii) the patient had lung cancer at the first point in time and the level which increases over time indicates that lung cancer is worsening in the patient,
(iii) the patient had lung cancer at the first point in time and the level which does not change over time indicates that lung cancer is not progressing in the patient, or
(iv) the patient had lung cancer at the first point in time and the level which decreases over time indicates that lung cancer is improving in the patient.

More specifically, the patient receives, has received, or had received a therapeutic treatment of lung cancer.

Even more specifically, the therapeutic treatment of lung cancer is selected from the group consisting of surgery, chemotherapy, targeted therapy, immunotherapy, oncolytic viral therapy, vaccination therapy, radiotherapy, laser therapy, hyperthermia therapy, and administration of a drug, or is a combination thereof.

In preferred embodiments (i), (ii), and/or (iii), the level of the RNA molecule selected from the group consisting of SEQ ID NO: 4, SEQ ID NO: 10, SEQ ID NO: 14, SEQ ID NO: 16, SEQ ID NO: 17, SEQ ID NO: 21, SEQ ID NO: 22, SEQ ID NO: 25, SEQ ID NO: 26, SEQ ID NO: 28, SEQ ID NO: 29, SEQ ID NO: 30, SEQ ID NO: 31, SEQ ID NO: 32, SEQ ID NO: 35, SEQ ID NO: 39, SEQ ID NO: 40, SEQ ID NO: 42, SEQ ID NO: 47, SEQ ID NO: 48, SEQ ID NO: 52, SEQ ID NO: 61, SEQ ID NO: 63, SEQ ID NO: 64, SEQ ID NO: 65, SEQ ID NO: 66, SEQ ID NO: 67, SEQ ID NO: 68, SEQ ID NO: 70, SEQ ID NO: 71, SEQ ID NO: 74, SEQ ID NO: 75, SEQ ID NO: 76, and/or SEQ ID NO: 79 which
(i) decreases over time indicates that the patient has developed lung cancer or that lung cancer is worsening in the patient,
(ii) does not change over time indicates that the patient is stable or that lung cancer is not progressing in the patient, or
(iii) increases over time indicates that lung cancer is improving in the patient.

Specifically,
(i) the patient was healthy at the first point in time and the level which decreases over time indicates that the patient has developed lung cancer,
(ii) the patient had lung cancer at the first point in time and the level which decreases over time indicates that lung cancer is worsening in the patient,
(iii) the patient had lung cancer at the first point in time and the level which does not change over time indicates that lung cancer is not progressing in the patient, or
(iv) the patient had lung cancer at the first point in time and the level which increases over time indicates that lung cancer is improving in the patient.

More specifically, the patient receives, has received, or had received a therapeutic treatment of lung cancer.

Even more specifically, the therapeutic treatment of lung cancer is selected from the group consisting of surgery, chemotherapy, targeted therapy, immunotherapy, oncolytic viral therapy, vaccination therapy, radiotherapy, laser therapy, hyperthermia therapy, and administration of a drug, or is a combination thereof.

In alternative or additional preferred embodiments (i), (ii), and/or (iii), the level of the at least one RNA molecule is specifically used in a mathematical computation to obtain a numerical value at the different points in time (i.e. first point in time and later point(s) in time) which are then compared with each other, or the levels of the at least two RNA molecules are specifically used in a mathematical computation to obtain a numerical value at the different points in time (i.e. first point in time and later point(s) in time) which are then compared with each other.

The mathematical computation is carried out as described above.

Specifically, the numerical value is obtained by summarizing the weighted levels of the at least two RNA molecules, e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 RNA molecules (such as 71 or 79 RNA molecules).

With the at least two RNA molecules, the RNA molecules having a nucleotide sequence according to SEQ ID NO: 1 and SEQ ID NO: 2 and optionally at least one RNA molecule having a nucleotide sequence according to SEQ ID NO: 3 to SEQ ID NO: 71, or the RNA molecules having a nucleotide sequence according to SEQ ID NO: 1 and SEQ ID NO: 2 and optionally at least one RNA molecule having a nucleotide sequence according to SEQ ID NO:

3 to SEQ ID NO: 79 are meant.

Alternatively, with the at least two RNA molecules, the RNA molecules having a nucleotide sequence according to SEQ ID NO: 2 and SEQ ID NO: 3 and optionally at least one RNA molecule having a nucleotide sequence according to SEQ ID NO: 1, SEQ ID NO: 4 to SEQ ID NO: 79 are meant.

The at least two RNA molecules can also be designated as RNA molecule signature.

Preferably, the numerical value is compared to an empirically determined cut-off score.

With respect to embodiment (i):
More preferably, the numerical value (z) is calculated for SEQ ID NO: 1 as follows:

$$z = f ( 1. * x 1 + - 5.24 ) ,$$

$$f ( x ) = 1 / ( 1 + exp ( - x ) ) ) ) ,$$

wherein x1 is the standardized log 2-RPM expression of SEQ ID NO 1, RPM stands for reads per million, the cut-off score (y) is 0.5, and
wherein the numerical value (z) is >0.5 and is further increasing over time which indicates that lung cancer worsens in the patient, or
wherein the numerical value (z) is >0.5 and is not further increasing over time which indicates that lung cancer is stable in the patient.

With respect to embodiment (ii):
More preferably, the numerical value (z) is calculated for the signature: SEQ ID NO: 1 and SEQ ID NO: 2 as follows:

$$z = f ( 0.95 * x 1 + 0.71 * x 2 + - 5.71 ) ,$$

$$f ( x ) = 1 / ( 1 + exp ( - x ) ) ) ) ,$$

wherein x1 and x2 is the standardized log 2-RPM expression of SEQ ID NO: 1 and SEQ ID NO: 2, RPM stands for reads per million, the cut-off score (y) is 0.5, and
wherein the numerical value (z) is >0.5 and is further increasing over time which indicates that lung cancer worsens in the patient, or
wherein the numerical value (z) is >0.5 and is not further increasing over time which indicates that lung cancer is stable in the patient.

Even more preferably, the numerical value (z) is calculated for the signature: SEQ ID NO: 1, SEQ ID NO: 2, SEQ ID NO: 19, SEQ ID NO: 35, SEQ ID NO: 39, and SEQ ID NO: 48 as follows:

$$z = f ( 0.94 * x 1 + 0.86 * x 2 + - 0.07 * x 19 + - 0.33 * x 35 + - 0.15 * x 39 + - 0.33 * x 48 + - 3.86 ) ,$$

$$f ( x ) = 1 / ( 1 + exp ( - x ) ) ) ) ,$$

wherein x1, x2, x19, x35, x39, and x48 is the standardized log 2-RPM expression of SEQ ID NO: 1, SEQ ID NO: 2, SEQ ID NO: 19, SEQ ID NO: 35, SEQ ID NO: 39, and SEQ ID NO: 48, respectively, RPM stands for reads per million, the cut-off score (y) is 0.5, and
wherein the numerical value (z) is >0.5 and is further increasing over time which indicates that lung cancer worsens in the patient, or
wherein the numerical value (z) is >0.5 and is not further increasing over time which indicates that lung cancer is stable in the patient.

Alternatively, the numerical value (z) is calculated for the signature: SEQ ID NO: 1, SEQ ID NO: 2, SEQ ID NO: 23, SEQ ID NO: 39, and SEQ ID NO: 48 as follows:

$$y = f ( 0.44 * x 1 + 0.83 * x 2 + 0.45 * x 23 + - 0.07 * x 39 + - 0.25 * x 48 + - 4.34 ) ,$$

$$f ( x ) = 1 / ( 1 + exp ( - x ) ) ) ) ,$$

wherein x1, x2, x23, x39, and x48 is the standardized log 2-RPM expression of SEQ ID NO: 1, SEQ ID NO: 2, SEQ ID NO: 23, SEQ ID NO: 39, and SEQ ID NO: 48, respectively, RPM stands for reads per million, the cut-off score (y) is 0.5, and
wherein the numerical value (z) is >0.5 and is further increasing over time which indicates that lung cancer worsens in the patient, or
wherein the numerical value (z) is >0.5 and is not further increasing over time which indicates that lung cancer is stable in the patient.

Alternatively, the numerical value (z) is calculated for the signature: SEQ ID NO: 1, SEQ ID NO: 2, SEQ ID NO: 6, SEQ ID NO: 7, and SEQ ID NO: 9 as follows:

$$z = f ( 0.84 * x 1 + 0.6 * x 2 + 0.85 * x 6 + 0.23 * x 7 + 0.3 * x 9 + - 6.44 ) ,$$

$$f ( x ) = 1 / ( 1 + exp ( - x ) ) ) ) ,$$

wherein x1, x2, x6, x7, and x9 is the standardized log 2-RPM expression of SEQ ID NO: 1, SEQ ID NO: 2, SEQ ID NO: 6, SEQ ID NO: 7, SEQ ID NO: 9, respectively, RPM stands for reads per million, the cut-off score (y) is 0.5, and
wherein the numerical value (z) is >0.5 and is further increasing over time which indicates that lung cancer worsens in the patient, or
wherein the numerical value (z) is >0.5 and is not further increasing over time which indicates that lung cancer is stable in the patient.

Alternatively, the numerical value (z) is calculated for the signature: SEQ ID NO: 1, SEQ ID NO: 2, SEQ ID NO: 6, SEQ ID NO: 7, and SEQ ID NO: 11, and SEQ ID NO: 18 as follows:

$$y = f ( 0.19 * x 1 + 0.62 * x 2 + 0.87 * x 6 + 0.32 * x 7 + 0.4 * x 11 + 0.65 * x 18 + - 6.12 ) ,$$

$$f ( x ) = 1 / ( 1 + exp ( - x ) ) ) ) ,$$

wherein x1, x2, x6, x7, x11, and x18 is the standardized log 2-RPM expression of SEQ ID NO: 1, SEQ ID NO: 2, SEQ ID NO: 6, SEQ ID NO: 7, SEQ ID NO: 11, and SEQ ID NO: 18, respectively, RPM stands for reads per million, the cut-off score (y) is 0.5, and
wherein the numerical value (z) is >0.5 and is further increasing over time which indicates that lung cancer worsens in the patient, or
wherein the numerical value (z) is >0.5 and is not further increasing over time which indicates that lung cancer is stable in the patient.

Still even more preferably, the numerical value (z) is calculated for the signature: SEQ ID NO: 1, SEQ ID NO: 2, SEQ ID NO: 19, SEQ ID NO: 35, SEQ ID NO: 38, SEQ ID NO: 39, SEQ ID NO: 40, SEQ ID NO: 43, SEQ ID NO: 48, and SEQ ID NO: 49 as follows:

$$z = f ( 0.93 * x 1 + 0.83 * x 2 + - 0.09 * x 19 + - 0.33 * x 35 + - 0.11 * x 38 + - 0.13 * x 39 + - 0.12 * x 40 + 0.17 * x 43 + - 0.29 * x 48 + 0.10 * x 49 + - 4.41 ) ,$$

$$f ( x ) = 1 / ( 1 + exp ( - x ) ) ) ) ,$$

wherein x1, x2, x19, x35, x38, x39, x40, x43, x48, and x49 is the standardized log 2-RPM expression of SEQ ID NO: 1, SEQ ID NO: 2, SEQ ID NO: 19, SEQ ID NO: 35, SEQ ID NO: 38, SEQ ID NO: 39, SEQ ID NO: 40, SEQ ID NO: 43, SEQ ID NO: 48, and SEQ ID NO: 49, respectively, RPM stands for reads per million, the cut-off score (y) is 0.5, and
wherein the numerical value (z) is >0.5 and is further increasing over time which indicates that lung cancer worsens in the patient, or
wherein the numerical value (z) is >0.5 and is not further increasing over time which indicates that lung cancer is stable in the patient.

Alternatively, the numerical value (z) is calculated for the signature: SEQ ID NO: 1, SEQ ID NO: 2, SEQ ID NO: 17, SEQ ID NO: 23, SEQ ID NO: 35, SEQ ID NO: 39, SEQ ID NO: 45, SEQ ID NO: 48, SEQ ID NO: 52, and SEQ ID NO: 53 as follows:

$$z = f ( 0.52 * x 1 + 0.93 * x 2 + - 0.22 * x 17 + 0.43 * x 23 + - 0.22 * x 35 + - 0.08 * x 39 + - 0.06 * x 45 + - 0.22 * x 48 + - 0.32 * x 52 + 0.02 * x 53 + - 1.32 ) ,$$

$$f ( x ) = 1 / ( 1 + exp ( - x ) ) ) ) ,$$

wherein x1, x2, x17, x23, x35, x39, x45, x48, x52, and x53 is the standardized log 2-RPM expression of SEQ ID NO: 1, SEQ ID NO: 2, SEQ ID NO: 17, SEQ ID NO: 23, SEQ ID NO: 35, SEQ ID NO: 39, SEQ ID NO: 45, SEQ ID NO: 48, SEQ ID NO: 52, and SEQ ID NO: 53, respectively, RPM stands for reads per million, the cut-off score (y) is 0.5, and
wherein the numerical value (z) is >0.5 and is further increasing over time which indicates that lung cancer worsens in the patient, or
wherein the numerical value (z) is >0.5 and is not further increasing over time which indicates that lung cancer is stable in the patient.

Alternatively, the numerical value (z) is calculated for the signature: SEQ ID NO: 1, SEQ ID NO: 2, SEQ ID NO: 6, SEQ ID NO: 7, SEQ ID NO: 9, SEQ ID NO: 19, SEQ ID NO: 25, SEQ ID NO: 29, SEQ ID NO: 30, and SEQ ID NO: 38 as follows:

$$z = f ( 0.83 * x 1 + 0.77 * x 2 + 0.69 * x 6 + 0.31 * x7 + 0.34 * x 9 + - 0.03 * x 19 + - 0.58 * x 25 + - 0.33 * x 29 + - 0.28 * x 30 + - 0.19 * x 38 + 3.17 ) ,$$

$$f ( x ) = 1 / ( 1 + exp ( - x ) ) ) ) ,$$

wherein x1, x2, x6, x7, x9, x19, x25, x29, x30, and x38 is the standardized log 2-RPM expression of SEQ ID NO: 1, SEQ ID NO: 2, SEQ ID NO: 6, SEQ ID NO: 7, SEQ ID NO: 9, SEQ ID NO: 19, SEQ ID NO: 25, SEQ ID NO: 29, SEQ ID NO: 30, and SEQ ID NO: 38, respectively, RPM stands for reads per million, the cut-off score (y) is 0.5, and
wherein the numerical value (z) is >0.5 and is further increasing over time which indicates that lung cancer worsens in the patient, or
wherein the numerical value (z) is >0.5 and is not further increasing over time which indicates that lung cancer is stable in the patient.

Alternatively, the numerical value (z) is calculated for the signature: SEQ ID NO: 1, SEQ ID NO: 2, SEQ ID NO: 3, SEQ ID NO: 6, SEQ ID NO: 7, SEQ ID NO: 11, SEQ ID NO: 12, SEQ ID NO: 14, SEQ ID NO: 18, and SEQ ID NO: 28 as follows:

$$z = f ( 0.24 * x 1 + 0.74 * x 2 + 0.19 * x 3 + 0.68 * x 6 + 0.36 * x 7 + 0.26 * x 11 + 0.26 * x 12 + - 0 \text{.42} * x 14 + 0.66 * x 18 + - 0.94 * x 28 + 4.76 ) ,$$

$$f ( x ) = 1 / ( 1 + exp ( - x ) ) ) ) ,$$

wherein x1, x2, x3, x6, x7, x11, x12, x14, x18, and x28 is the standardized log 2-RPM expression of SEQ ID NO: 1, SEQ ID NO: 2, SEQ ID NO: 3, SEQ ID NO: 6, SEQ ID NO: 7, SEQ ID NO: 11, SEQ ID NO: 12, SEQ ID NO: 14, SEQ ID NO: 18, and SEQ ID NO: 28, respectively, RPM stands for reads per million, the cut-off score (y) is 0.5, and
wherein the numerical value (z) is >0.5 and is further increasing over time which indicates that lung cancer worsens in the patient, or
wherein the numerical value (z) is >0.5 and is not further increasing over time which indicates that lung cancer is stable in the patient.

Most preferably, the numerical value (z) is calculated for the (54) signature: SEQ ID NO: 1 to SEQ ID NO: 54 as follows: z=f(0.45*x1+0.83*x2+0.74*x3+0.50*x4+−0.48*x5+0.74*x6+0.48*x7+0.55*x8+0.64*x9+−0.72*x10+0.39*x11+0.25*x12+0.35*x13+−0.34*x14+−0.43*x15+−0.40*x16+−0.34*x17+0.14*x18+−0.32*x19+−0.52*x20+−0.28*x21+−0.32*x22+−0.28*x23+0.20*x24+−0.33*x25+−0.24*x26+0.26*x27+0.44*x28+−0.11*x29+−0.12*x30+−0.10*x31+0.25*x32+−0.26*x33+0.16*x34+−0.15*x35+−0.24*x36+0.10*x37+−0.11*x38+−0.08*x39+0.08*x40+0.05*x41+−0.10*x42+0.06*x43+0.06*x44+0.12*x45+−0.06*x46+0.10*x47+−0.02*x48+0.01*x49+−0.04*x50+0.02*x51+0.01*x52+0.04*x53+0.05*x54+0.21), f(x)=1/(1+exp(−x)))),
wherein x1 to x54 is the standardized log 2-RPM expression of SEQ ID NO: 1 to SEQ ID NO: 54, respectively, RPM stands for reads per million, the cut-off score (y) is 0.5, and
wherein the numerical value (z) is >0.5 and is further increasing over time which indicates that lung cancer worsens in the patient, or
wherein the numerical value (z) is >0.5 and is not further increasing over time which indicates that lung cancer is stable in the patient.

Alternatively, the numerical value (z) is calculated for the (38) signature: SEQ ID NO: 1, SEQ ID NO: 2, SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, and SEQ ID NO: 71 as follows:

$$z = f ( 0.05 * x 1 + 0.83 * x 2 + 0.27 * x 3 + 0.89 * x 6 + 0.35 * x 7 + 0.29 * x 8 + - 0.56 * x 10 + 0.14 * x 11 + 0.13 * x 12 + - 0.16 * x 14 + - 0.21 * x 16 + - 0.29 * x 17 + 0.37 * x 18 + - 0.21 * x 21 + - 0.26 * x 22 + - 0.15 * x 24 + - 0.26 * x 26 + - 0.74 * x 28 + - 0.17 * x 29 + - 0.17 * x 31 + 0.17 * x 34 + 0.16 * x 55 + 0.18 * x 56 + 0.19 * x 57 + 0.08 * x 58 + 0.27 * x 59 + 0.39 * x 60 + 0.5 * x 61 + 0.24 * x 62 + - 0.1 * x 63 + - 0.19 * x 64 + - 0.12 * x 65 + - 0.54 * x 66 + - 0.07 * x 67 + - 0.2 * x 68 + - 0.25 * x 69 + 0.81 * x 70 + - 0.19 * x 71 + 0.33 ) ,$$

$$f ( x ) = 1 / ( 1 + exp ( - x ) ) ) ) ,$$

wherein x1, x2, x3, x6, x7, x8, x10, x11, x12, x14, x16, x17, x18, x21, x22, x24, x26, x28, x29, x31, x34, x55 to x71 is the standardized log 2-RPM expression of SEQ ID NO: 1, SEQ ID NO: 2, SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, and SEQ ID NO: 71, respectively, RPM stands for reads per million, the cut-off score (y) is 0.5, and
wherein the numerical value (z) is >0.5 and is further increasing over time which indicates that lung cancer worsens in the patient, or
wherein the numerical value (z) is >0.5 and is not further increasing over time which indicates that lung cancer is stable in the patient.

With respect to embodiment (iii):
More preferably, the numerical value (z) is calculated for the (2) signature: SEQ ID NO: 2 and SEQ ID NO: 3 as follows:

$$z = f ( 0.800 * x 2 + 1.342 * x 3 + - 8.733 ) , f ( x ) = 1 \underline{/} ( 1 + exp ( - x ) ) ) ) ,$$

wherein x2, x3 is the log 2-RPM expression of SEQ ID NO: 2, SEQ ID NO: 3, respectively, RPM stands for reads per million, the cut-off score (y) is 0.5, and
wherein the numerical value (z) is >0.5 and is further increasing over time which indicates that lung cancer worsens in the patient, or
wherein the numerical value (z) is >0.5 and is not further increasing over time which indicates that lung cancer is stable in the patient.

Even more preferably, the numerical value (z) is calculated for the (18) signature: SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, and SEQ ID NO: 79 as follows:

$$z = f ( 0.792 * x 2 + 0.54 * x 3 + 0.521 * x 7 + 0.26 * x 11 + - 0.284 * x 14 + - 0.306 * x 17 + 0.399 * x 18 + - 0.429 * x 21 + - 0.693 * x 28 + 0.295 * x 56 + 0.452 * x 72 + 0.38 * x 73 + - 0.3 * x 74 + - 0.297 * x 75 + - 0.219 * x 76 + 0.157 * x 77 + 0 \text{.0658} * x 78 + - 0.0421 * x 79 + 5.203 ) ,$$

$$f ( x ) = 1 \underline{/} ( 1 + exp ( - x ) ) ) ) ,$$

wherein x2, x3, x7, x11, x14, x17, x18, x21, x28, x56, x72, x73, x74, x75, x76, x77, x78, x79 is the log 2-RPM expression of SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, respectively, RPM stands for reads per million, the cut-off score (y) is 0.5, and
wherein the numerical value (z) is >0.5 and is further increasing over time which indicates that lung cancer worsens in the patient, or
wherein the numerical value (z) is >0.5 and is not further increasing over time which indicates that lung cancer is stable in the patient.

Even more preferably, the numerical value (z) is calculated for the (6) signature (Mitra samples):
SEQ ID NO: 2, SEQ ID NO: 3, SEQ ID NO: 7, SEQ ID NO: 17, SEQ ID NO: 18, and SEQ ID NO: 75 as follows:

Even more preferably, the numerical value (z) is calculated for the (6) signature (Mitra samples):
SEQ ID NO: 2, SEQ ID NO: 3, SEQ ID NO: 7, SEQ ID NO: 17, SEQ ID NO: 18, and SEQ ID NO: 75 as follows:

$$z = f ( 19.621 * x 2 + 0.638 * x 3 + 1.171 * x 7 + - 0.492 * x 17 + - 2.4 * x 18 + 1.049 * x 75 + 3.185 ) ,$$

$$f ( x ) = 1 \underline{/} ( 1 + exp ( - x ) ) ) ) ,$$

wherein x2, x3, x7, x17, x18, x75 is the log 2-RPM expression of SEQ ID NO: 2, SEQ ID NO: 3, SEQ ID NO: 7, SEQ ID NO: 17, SEQ ID NO: 18, SEQ ID NO: 75, respectively, RPM stands for reads per million, the cut-off score (y) is 0.5, and
wherein the numerical value (z) is >0.5 and is further increasing over time which indicates that lung cancer worsens in the patient, or wherein the numerical value (z) is >0.5 and is not further increasing over time which indicates that lung cancer is stable in the patient.

The patient may receive a therapeutic treatment during the complete monitoring process (e.g. the administration of a drug) or may receive a therapeutic treatment before, at, or after a first point in time (e.g. the administration of a drug) and may be retested at a later point in time. In particular, said first point in time may be before the initiation of a therapeutic treatment and said later point in time may be during the therapeutic treatment and/or after the therapeutic treatment. If the therapeutic treatment encompasses the administration of a drug and the patient responds to said treatment, the drug administration may be continued, the dose of the drug may be reduced, or the drug administration may be stopped. If the therapeutic treatment encompasses the administration of a drug and the patient does not respond to said treatment, the dose of the drug may be increased, the drug may be changed, or the therapy mode may be changed, e.g. from drug administration to surgery, laser therapy, or hyperthermia therapy.

Due to the therapeutic treatment, an increased level of the RNA molecule can be decreased and/or a decreased level of the RNA molecule can be increased. Alternatively, due to the therapeutic treatment, an increased numerical value of the RNA molecules can be decreased and/or a decreased numerical value of the RNA molecules can be increased. In this way, the (overall) condition of the patient having lung cancer can be improved.

It is preferred in the method of the first aspect that the lung cancer is lung cancer of stage I or II (i.e. early-stage lung cancer). A diagnostic test in this respect can significantly reduce mortality as the lung cancer is detectable very early. It is further preferred that the lung cancer is small-cell lung carcinoma (SCLC) or non-small-cell lung carcinoma (NSCLC), particularly lung adenocarcinoma (LUAD) or lung squamous cell carcinoma (LUSC), e.g. small-cell lung carcinoma (SCLC) or non-small-cell lung carcinoma (NSCLC), particularly lung adenocarcinoma (LUAD) or lung squamous cell carcinoma (LUSC), of stage I or II.

In the method of the second aspect, the lung cancer is preferably small-cell lung carcinoma (SCLC) or non-small-cell lung carcinoma (NSCLC), more preferably lung adenocarcinoma (LUAD) or lung squamous cell carcinoma (LUSC).

In the methods of the first or second aspect, the patient is preferably a mammal, more preferably a human or a rodent, even more preferably a human.

In the methods of the first or second aspect, the blood sample is preferably whole blood or a blood fraction. More preferably, the blood fraction is selected from the group consisting of a blood cell fraction, plasma, and serum. Even more preferably, the blood cell fraction is a fraction of/comprising erythrocytes, leukocytes, and/or thrombocytes.

In the methods of the first or second aspect, the level of the RNA molecule is preferably determined by sequencing, specifically next generation sequencing (e.g. ABI SOLID, Illumina Genome Analyzer, Roche 454 GS FL, BGISEQ), nucleic acid hybridization (e.g. microarray or beads), nucleic acid amplification (e.g. PCR, RT-PCR, qRT-PCR, or high-throughput RT-PCR), polymerase extension, mass spectrometry, flow cytometry (e.g. LUMINEX), or any combination thereof.

In the methods of the first or second aspect, the level of the RNA molecule is the expression level of said RNA molecule.

In a third aspect, the present invention relates to the (in vitro) use of
(i) at least one RNA molecule for diagnosing lung cancer in a patient, or for monitoring a patient suspected of having lung cancer or a patient suffering from lung cancer, wherein the at least one RNA molecule comprises/has a nucleotide sequence according to SEQ ID NO: 1, is a fragment thereof, or has a sequence having at least 80% sequence identity thereto,
(ii) at least two RNA molecules for diagnosing lung cancer in a patient, or for monitoring a patient suspected of having lung cancer or a patient suffering from lung cancer, wherein the at least two RNA molecules comprise a first RNA molecule and a second RNA molecule, wherein the first RNA molecule comprises/has a nucleotide sequence according to SEQ ID NO: 1, is a fragment thereof, or has a sequence having at least 80% sequence identity thereto and the second RNA molecule comprises/has a nucleotide sequence according to SEQ ID NO: 2, is a fragment thereof, or has a sequence having at least 80% sequence identity thereto, or
(iii) at least two RNA molecules for diagnosing lung cancer in a patient, or for monitoring a patient suspected of having lung cancer or a patient suffering from lung cancer, wherein the at least two RNA molecules comprise a first RNA molecule and a second RNA molecule,
   wherein the first RNA molecule comprises a nucleotide sequence according to SEQ ID NO: 2, is a fragment thereof, or has a sequence having at least 80% sequence identity thereto and the second RNA molecule comprises a nucleotide sequence according to SEQ ID NO: 3, is a fragment thereof, or has a sequence having at least 80% sequence identity thereto.

Preferably,
the first RNA molecule comprises a nucleotide sequence according to SEQ ID NO: 1 and the second RNA molecule comprises a nucleotide sequence according to SEQ ID NO: 2, or the first RNA molecule comprises a nucleotide sequence according to SEQ ID NO: 2 and the second RNA molecule comprises a nucleotide sequence according to SEQ ID NO: 3.

Especially, the at least two RNA molecules are comprised in a set. In other words, the at least two RNA molecules represent a signature. The signature can encompass, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 RNA molecules, specifically 2, 5, 6, 10, 18, 38, or 54 RNA molecules (RNA signatures).

In embodiment (i),
at least one further RNA molecule selected from the group consisting of an RNA molecule comprising/having a nucleotide sequence according to SEQ ID NO: 2 to SEQ ID NO: 71, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto is used for diagnosing or monitoring lung cancer in the patient, or
at least one further RNA molecule selected from the group consisting of an RNA molecule comprising/having a nucleotide sequence according to SEQ ID NO: 2 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto is used for diagnosing or monitoring lung cancer in the patient.

Thus, the RNA molecule comprising a nucleotide sequence according to SEQ ID NO: 1, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and at least one further RNA molecule selected from the group consisting of an RNA molecule comprising a nucleotide sequence according to SEQ ID NO: 2 to SEQ ID NO: 71, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are used for diagnosing or monitoring lung cancer in the patient, or
at least one further RNA molecule selected from the group consisting of an RNA molecule comprising a nucleotide sequence according to SEQ ID NO: 2 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are used for diagnosing or monitoring lung cancer in the patient.

Specifically, the RNA molecule is an RNA molecule comprising
(i) a nucleotide sequence according to SEQ ID NO: 1 to SEQ ID NO: 71 or SEQ ID NO: 1 to SEQ ID NO: 79,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iii) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

In embodiment (ii),
at least one further RNA molecule selected from the group consisting of an RNA molecule comprising/having a nucleotide sequence according to SEQ ID NO: 3 to SEQ ID NO: 71, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto is used for diagnosing or monitoring lung cancer in the patient, or
at least one further RNA molecule selected from the group consisting of an RNA molecule comprising/having a nucleotide sequence according to SEQ ID NO: 3 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto is used for diagnosing or monitoring lung cancer in the patient.

Thus, the RNA molecule comprising a nucleotide sequence according to SEQ ID NO: 1, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto as well as the RNA molecule comprising a nucleotide sequence according to SEQ ID NO: 2, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and
the level of at least one further RNA molecule selected from the group consisting of an RNA molecule comprising a nucleotide sequence according to SEQ ID NO: 3 to SEQ ID NO: 71, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are used for diagnosing or monitoring lung cancer in the patient, or
the level of at least one further RNA molecule selected from the group consisting of an RNA molecule comprising a nucleotide sequence according to SEQ ID NO: 3 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are used for diagnosing or monitoring lung cancer in the patient.

Specifically, the RNA molecule is an RNA molecule comprising
(i) a nucleotide sequence according to SEQ ID NO: 1 to SEQ ID NO: 71 or SEQ ID NO: 1 to SEQ ID NO: 79,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iii) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

Preferably,
(i) at least one further RNA molecule selected from the group consisting of an RNA molecule comprising a nucleotide sequence according to SEQ ID NO: 19, SEQ ID NO: 35, SEQ ID NO: 39, SEQ ID NO: 48, a fragment thereof, and a sequence having at least 80% sequence identity thereto is used (for diagnosing or monitoring lung cancer in the patient),
(ii) at least one further RNA molecule selected from the group consisting of an RNA molecule comprising a nucleotide sequence according to SEQ ID NO: 23, SEQ ID NO: 39, SEQ ID NO: 48, a fragment thereof, and a sequence having at least 80% sequence identity thereto is used (for diagnosing or monitoring lung cancer in the patient),
(iii) at least one further RNA molecule selected from the group consisting of an RNA molecule comprising a nucleotide sequence according to SEQ ID NO: 6, SEQ ID NO: 7, SEQ ID NO: 9, a fragment thereof, and a sequence having at least 80% sequence identity thereto is used (for diagnosing or monitoring lung cancer in the patient),
(iv) at least one further RNA molecule selected from the group consisting of an RNA molecule comprising a nucleotide sequence according to SEQ ID NO: 6, SEQ ID NO: 7, SEQ ID NO: 11, SEQ ID NO: 18, a fragment thereof, and a sequence having at least 80% sequence identity thereto is used (for diagnosing or monitoring lung cancer in the patient), (v) at least one further RNA molecule selected from the group consisting of an RNA molecule comprising a nucleotide sequence according to SEQ ID NO: 19, SEQ ID NO: 35, SEQ ID NO: 38, SEQ ID NO: 39, SEQ ID NO: 40, SEQ ID NO: 43, SEQ ID NO: 48, SEQ ID NO: 49, a fragment thereof, and a sequence having at least 80% sequence identity thereto is used (for diagnosing or monitoring lung cancer in the patient),
(vi) at least one further RNA molecule selected from the group consisting of an RNA molecule comprising a nucleotide sequence according to SEQ ID NO: 17, SEQ ID NO: 23, SEQ ID NO: 35, SEQ ID NO: 39, SEQ ID NO: 45, SEQ ID NO: 48, SEQ ID NO: 52, SEQ ID NO: 53, a fragment thereof, and a sequence having at least 80% sequence identity thereto is used (for diagnosing or monitoring lung cancer in the patient),
(vii) at least one further RNA molecule selected from the group consisting of an RNA molecule comprising a nucleotide sequence according to SEQ ID NO: 6, SEQ ID NO: 7, SEQ ID NO: 9, SEQ ID NO: 19, SEQ ID NO: 25, SEQ ID NO: 29, SEQ ID NO: 30, SEQ ID NO: 38, a fragment thereof, and a sequence having at least 80% sequence identity thereto is used (for diagnosing or monitoring lung cancer in the patient),
(viii) at least one further RNA molecule selected from the group consisting of an RNA molecule comprising a nucleotide sequence according to SEQ ID NO: 3, SEQ ID NO: 6, SEQ ID NO: 7, SEQ ID NO: 11, SEQ ID NO: 12, SEQ ID NO: 14, SEQ ID NO: 18, SEQ ID NO: 28, a fragment thereof, and a sequence having at least 80% sequence identity thereto is used (for diagnosing or monitoring lung cancer in the patient),
(ix) at least one further RNA molecule selected from the group consisting of an RNA molecule comprising a nucleotide sequence according to SEQ ID NO: 3 to SEQ ID NO: 54, a fragment thereof, and a sequence having at least 80% sequence identity thereto is used (for diagnosing or monitoring lung cancer in the patient), or
(x) at least one further RNA molecule selected from the group consisting of an RNA molecule comprising a nucleotide sequence according to SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, a fragment thereof, and a sequence having at least 80% sequence identity thereto is used (for diagnosing or monitoring lung cancer in the patient).

More preferably, the RNA molecules comprising a nucleotide sequence according to
(i) SEQ ID NO: 1, SEQ ID NO: 2, SEQ ID NO: 19, SEQ ID NO: 35, SEQ ID NO: 39, and SEQ ID NO: 48,
(ii) SEQ ID NO: 1, SEQ ID NO: 2, SEQ ID NO: 23, SEQ ID NO: 39, and SEQ ID NO: 48,
(iii) SEQ ID NO: 1, SEQ ID NO: 2, SEQ ID NO: 6, SEQ ID NO: 7, and SEQ ID NO: 9,
(iv) SEQ ID NO: 1, SEQ ID NO: 2, SEQ ID NO: 6, SEQ ID NO: 7, SEQ ID NO: 11, and SEQ ID NO: 18,
(v) SEQ ID NO: 1, SEQ ID NO: 2, SEQ ID NO: 19, SEQ ID NO: 35, SEQ ID NO: 38, SEQ ID NO: 39, SEQ ID NO: 40, SEQ ID NO: 43, SEQ ID NO: 48, and SEQ ID NO: 49,
(vi) SEQ ID NO: 1, SEQ ID NO: 2, SEQ ID NO: 17, SEQ ID NO: 23, SEQ ID NO: 35, SEQ ID NO: 39, SEQ ID NO: 45, SEQ ID NO: 48, SEQ ID NO: 52, and SEQ ID NO: 53,
(vii) SEQ ID NO: 1, SEQ ID NO: 2, SEQ ID NO: 6, SEQ ID NO: 7, SEQ ID NO: 9, SEQ ID NO: 19, SEQ ID NO: 25, SEQ ID NO: 29, SEQ ID NO: 30, and SEQ ID NO: 38,
(viii) SEQ ID NO: 1, SEQ ID NO: 2, SEQ ID NO: 3, SEQ ID NO: 6, SEQ ID NO: 7, SEQ ID NO: 11, SEQ ID NO: 12, SEQ ID NO: 14, SEQ ID NO: 18, and SEQ ID NO: 28,
(ix) SEQ ID NO: 1 to SEQ ID NO: 54, or
(x) SEQ ID NO: 1, SEQ ID NO: 2, SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, and SEQ ID NO: 71
are used for diagnosing or monitoring lung cancer in the patient.

In embodiment (iii),
at least one further RNA molecule selected from the group consisting of an RNA molecule comprising/having a nucleotide sequence according to SEQ ID NO: 1, SEQ ID NO: 4 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto is used for diagnosing or monitoring lung cancer in the patient.

Thus, the RNA molecule comprising a nucleotide sequence according to SEQ ID NO: 2, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto as well as the RNA molecule comprising a nucleotide sequence according to SEQ ID NO: 3, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and the level of at least one further RNA molecule selected from the group consisting of an RNA molecule comprising a nucleotide sequence according to SEQ ID NO: 1, SEQ ID NO: 4 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are used for diagnosing or monitoring lung cancer in the patient.

Specifically, the RNA molecule is an RNA molecule comprising
(i) a nucleotide sequence according to SEQ ID NO: 1 to SEQ ID NO: 79,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iii) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

Preferably,
(i) at least one further RNA molecule selected from the group consisting of an RNA molecule comprising a nucleotide sequence according to SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72 to SEQ ID NO: 79, a fragment thereof, and a sequence having at least 80% sequence identity thereto is used for diagnosing or monitoring lung cancer in the patient, or
(ii) at least one further RNA molecule selected from the group consisting of an RNA molecule comprising a nucleotide sequence according to SEQ ID NO: 7, SEQ ID NO: 17, SEQ ID NO: 18, SEQ ID NO: 75, a fragment thereof, and a sequence having at least 80% sequence identity thereto is used for diagnosing or monitoring lung cancer in the patient.

More preferably, the RNA molecules comprising a nucleotide sequence according to
(i) SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, and SEQ ID NO: 79, or
(ii) SEQ ID NO: 2, SEQ ID NO: 3, SEQ ID NO: 7, SEQ ID NO: 17, SEQ ID NO: 18, and SEQ ID NO: 75
are used for diagnosing or monitoring lung cancer in the patient.

The diagnosis or the monitoring is specifically carried out in a blood sample from the patient. For this purpose, the level the RNA molecule(s) is (are) specifically determined/analyzed in the blood sample from the patient. Preferably, the blood sample is whole blood or a blood fraction. More preferably, the blood fraction is selected from the group consisting of a blood cell fraction, plasma, and serum. Even more preferably, the blood cell fraction is a fraction of/comprising erythrocytes, leukocytes, and/or thrombocytes.

It is preferred that the lung cancer is lung cancer of stage I or II (i.e. early-stage lung cancer). It is further preferred that the lung cancer is small-cell lung carcinoma (SCLC) or non-small-cell lung carcinoma (NSCLC), particularly lung adenocarcinoma (LUAD) or lung squamous cell carcinoma (LUSC), e.g. small-cell lung carcinoma (SCLC) or non-small-cell lung carcinoma (NSCLC), particularly lung adenocarcinoma (LUAD) or lung squamous cell carcinoma (LUSC), of stage I or II.

As to other preferred embodiments, it is referred to the first or second aspect of the present invention.

In a fourth aspect, the present invention relates to (the (in vitro) use of) a kit for diagnosing lung cancer in a patient, or for monitoring a patient suspected of having lung cancer or a patient suffering from lung cancer, said kit comprises
(i) means for determining the level of at least one RNA molecule in a blood sample from a patient,
wherein the at least one RNA molecule comprises/has a nucleotide sequence according to SEQ ID NO: 1, is a fragment thereof, or has a sequence having at least 80% sequence identity thereto,
(ii) means for determining the levels of at least two RNA molecules in a blood sample from a patient, wherein the at least two RNA molecules comprise a first RNA molecule and a second RNA molecule,
wherein the first RNA molecule comprises/has a nucleotide sequence according to SEQ ID NO: 1, is a fragment thereof, or has a sequence having at least 80% sequence identity thereto and the second RNA molecule comprises/has a nucleotide sequence according to SEQ ID NO: 2, is a fragment thereof, or has a sequence having at least 80% sequence identity thereto, or
(iii) means for determining the levels of at least two RNA molecules in a blood sample from a patient, wherein the at least two RNA molecules comprise a first RNA molecule and a second RNA molecule,
wherein the first RNA molecule comprises a nucleotide sequence according to SEQ ID NO: 2, is a fragment thereof, or has a sequence having at least 80% sequence identity thereto and the second RNA molecule comprises a nucleotide sequence according to SEQ ID NO: 3, is a fragment thereof, or has a sequence having at least 80% sequence identity thereto.

Preferably,
the first RNA molecule comprises a nucleotide sequence according to SEQ ID NO: 1 and the second RNA molecule comprises a nucleotide sequence according to SEQ ID NO: 2, or
the first RNA molecule comprises a nucleotide sequence according to SEQ ID NO: 2 and the second RNA molecule comprises a nucleotide sequence according to SEQ ID NO: 3.

Especially, the at least two RNA molecules are comprised in a set. In other words, the at least two RNA molecules represent a signature. The signature can encompass, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79 RNA molecules, specifically 2, 5, 6, 10, 18, 38, or 54 RNA molecules (RNA signature).

In embodiment (i),
the kit comprises means for determining the level of at least one further RNA molecule selected from the group consisting of an RNA molecule comprising/having a nucleotide sequence according to SEQ ID NO: 2 to SEQ ID NO: 71, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto, or
the kit comprises means for determining the level of at least one further RNA molecule selected from the group consisting of an RNA molecule comprising/having a nucleotide sequence according to SEQ ID NO: 2 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Thus, the kit comprises means for determining the level of the RNA molecule comprising a nucleotide sequence according to SEQ ID NO: 1, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and
means for determining the level of at least one further RNA molecule selected from the group consisting of an RNA molecule comprising a nucleotide sequence according to SEQ ID NO: 2 to SEQ ID NO: 71, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto, or means for determining the level of at least one further RNA molecule selected from the group consisting of an RNA molecule comprising/having a nucleotide sequence according to SEQ ID NO: 2 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Specifically, the RNA molecule is an RNA molecule comprising
(i) a nucleotide sequence according to SEQ ID NO: 1 to SEQ ID NO: 71 or SEQ ID NO: 1 to SEQ ID NO: 79,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iii) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

In embodiment (ii),
the kit comprises means for determining the level of at least one further RNA molecule selected from the group consisting of an RNA molecule comprising/having a nucleotide sequence according to SEQ ID NO: 3 to SEQ ID NO: 71, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto, or
the kit comprises means for determining the level of at least one further RNA molecule selected from the group consisting of an RNA molecule comprising/having a nucleotide sequence according to SEQ ID NO: 3 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Thus, the kit comprises means for determining the level of the RNA molecule comprising a nucleotide sequence according to SEQ ID NO: 1, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto as well as means for determining the level of the RNA molecule comprising a nucleotide sequence according to SEQ ID NO: 2, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and means for determining the level of at least one further RNA molecule selected from the group consisting of an RNA molecule comprising a nucleotide sequence according to SEQ ID NO: 3 to SEQ ID NO: 71, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto, or means for determining the level of at least one further RNA molecule selected from the group consisting of an RNA molecule comprising a nucleotide sequence according to SEQ ID NO: 3 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Specifically, the RNA molecule is an RNA molecule comprising
(i) a nucleotide sequence according to SEQ ID NO: 1 to SEQ ID NO: 71, or SEQ ID NO: 1 to SEQ ID NO: 79,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iii) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

Preferably, the kit comprises means for determining the level of
(i) at least one further RNA molecule selected from the group consisting of an RNA molecule comprising a nucleotide sequence according to SEQ ID NO: 19, SEQ ID NO: 35, SEQ ID NO: 39, SEQ ID NO: 48, a fragment thereof, and a sequence having at least 80% sequence identity thereto,
(ii) at least one further RNA molecule selected from the group consisting of an RNA molecule comprising a nucleotide sequence according to SEQ ID NO: 23, SEQ ID NO: 39, SEQ ID NO: 48, a fragment thereof, and a sequence having at least 80% sequence identity thereto,
(iii) at least one further RNA molecule selected from the group consisting of an RNA molecule comprising a nucleotide sequence according to SEQ ID NO: 6, SEQ ID NO: 7, SEQ ID NO: 9, a fragment thereof, and a sequence having at least 80% sequence identity thereto,
(iv) at least one further RNA molecule selected from the group consisting of an RNA molecule comprising a nucleotide sequence according to SEQ ID NO: 6, SEQ ID NO: 7, SEQ ID NO: 11, SEQ ID NO: 18, a fragment thereof, and a sequence having at least 80% sequence identity thereto,
(v) at least one further RNA molecule selected from the group consisting of an RNA molecule comprising a nucleotide sequence according to SEQ ID NO: 19, SEQ ID NO: 35, SEQ ID NO: 38, SEQ ID NO: 39, SEQ ID NO: 40, SEQ ID NO: 43, SEQ ID NO: 48, SEQ ID NO: 49, a fragment thereof, and a sequence having at least 80% sequence identity thereto,
(vi) at least one further RNA molecule selected from the group consisting of an RNA molecule comprising a nucleotide sequence according to SEQ ID NO: 17, SEQ ID NO: 23, SEQ ID NO: 35, SEQ ID NO: 39, SEQ ID NO: 45, SEQ ID NO: 48, SEQ ID NO: 52, SEQ ID NO: 53, a fragment thereof, and a sequence having at least 80% sequence identity thereto,
(vii) at least one further RNA molecule selected from the group consisting of an RNA molecule comprising a nucleotide sequence according to SEQ ID NO: 6, SEQ ID NO: 7, SEQ ID NO: 9, SEQ ID NO: 19, SEQ ID NO: 25, SEQ ID NO: 29, SEQ ID NO: 30, SEQ ID NO: 38, a fragment thereof, and a sequence having at least 80% sequence identity thereto,
(viii) at least one further RNA molecule selected from the group consisting of an RNA molecule comprising a nucleotide sequence according to SEQ ID NO: 3, SEQ ID NO: 6, SEQ ID NO: 7, SEQ ID NO: 11, SEQ ID NO: 12, SEQ ID NO: 14, SEQ ID NO: 18, SEQ ID NO: 28, a fragment thereof, and a sequence having at least 80% sequence identity thereto,
(ix) at least one further RNA molecule selected from the group consisting of an RNA molecule comprising a nucleotide sequence according to SEQ ID NO: 3 to SEQ ID NO: 54, a fragment thereof, and a sequence having at least 80% sequence identity thereto is used, or
(x) at least one further RNA molecule selected from the group consisting of an RNA molecule comprising a nucleotide sequence according to SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, a fragment thereof, and a sequence having at least 80% sequence identity thereto.

More preferably, the kit comprises means for determining the levels of the RNA molecules comprising a nucleotide sequence according to
(i) SEQ ID NO: 1, SEQ ID NO: 2, SEQ ID NO: 19, SEQ ID NO: 35, SEQ ID NO: 39, and SEQ ID NO: 48,
(ii) SEQ ID NO: 1, SEQ ID NO: 2, SEQ ID NO: 23, SEQ ID NO: 39, and SEQ ID NO: 48,
(iii) SEQ ID NO: 1, SEQ ID NO: 2, SEQ ID NO: 6, SEQ ID NO: 7, and SEQ ID NO: 9,
(iv) SEQ ID NO: 1, SEQ ID NO: 2, SEQ ID NO: 6, SEQ ID NO: 7, SEQ ID NO: 11, and SEQ ID NO: 18,
(v) SEQ ID NO: 1, SEQ ID NO: 2, SEQ ID NO: 19, SEQ ID NO: 35, SEQ ID NO: 38, SEQ ID NO: 39, SEQ ID NO: 40, SEQ ID NO: 43, SEQ ID NO: 48, and SEQ ID NO: 49, SEQ ID NO: 1, SEQ ID NO: 2, SEQ ID NO: 17, SEQ ID NO: 23, SEQ ID NO: 35, SEQ (vi) ID NO: 39, SEQ ID NO: 45, SEQ ID NO: 48, SEQ ID NO: 52, and SEQ ID NO: 53,
(vii) SEQ ID NO: 1, SEQ ID NO: 2, SEQ ID NO: 6, SEQ ID NO: 7, SEQ ID NO: 9, SEQ ID NO: 19, SEQ ID NO: 25, SEQ ID NO: 29, SEQ ID NO: 30, and SEQ ID NO: 38,
(viii) SEQ ID NO: 1, SEQ ID NO: 2, SEQ ID NO: 3, SEQ ID NO: 6, SEQ ID NO: 7, SEQ ID NO: 11, SEQ ID NO: 12, SEQ ID NO: 14, SEQ ID NO: 18, and SEQ ID NO: 28,
(ix) SEQ ID NO: 1 to SEQ ID NO: 54, or
(x) SEQ ID NO: 1, SEQ ID NO: 2, SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, and SEQ ID NO: 71.

In embodiment (iii), the kit comprises means for determining the level of at least one further RNA molecule selected from the group consisting of an RNA molecule comprising/having a nucleotide sequence according to SEQ ID NO: 1, SEQ ID NO: 4 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Thus, the kit comprises means for determining the level of the RNA molecule comprising a nucleotide sequence according to SEQ ID NO: 2, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto as well as means for determining the level of the RNA molecule comprising a nucleotide sequence according to SEQ ID NO: 3, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and means for determining the level of at least one further RNA molecule selected from the group consisting of an RNA molecule comprising a nucleotide sequence according to SEQ ID NO: 1, SEQ ID NO: 4 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

Specifically, the RNA molecule is an RNA molecule comprising
(i) a nucleotide sequence according to SEQ ID NO: 1 to SEQ ID NO: 79,
(ii) a nucleotide sequence that is a fragment of the nucleotide sequence according to (i), preferably, a nucleotide sequence that is a fragment which is between 1 and 12, more preferably between 1 and 8, and most preferably between 1 and 5 or 1 and 3, e.g. 1, 2, 3, 4, 5, 6, 7, 8, 9, 10, 11, or 12, nucleotides shorter than the nucleotide sequence according to (i), or
(iii) a nucleotide sequence that has at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

Preferably, the kit comprises means for determining the level of
(i) at least one further RNA molecule selected from the group consisting of an RNA molecule comprising a nucleotide sequence according to SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72 to SEQ ID NO: 79, a fragment thereof, and a sequence having at least 80% sequence identity thereto, or
(ii) at least one further RNA molecule selected from the group consisting of an RNA molecule comprising a nucleotide sequence according to SEQ ID NO: 7, SEQ ID NO: 17, SEQ ID NO: 18, SEQ ID NO: 75, a fragment thereof, and a sequence having at least 80% sequence identity thereto.

More preferably, the kit comprises means for determining the levels of the RNA molecules comprising a nucleotide sequence according to
(i) SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, and SEQ ID NO: 79, or
(ii) SEQ ID NO: 2, SEQ ID NO: 3, SEQ ID NO: 7, SEQ ID NO: 17, SEQ ID NO: 18, and SEQ ID NO: 75.

Preferably, the blood sample is whole blood or a blood fraction. More preferably, the blood fraction is selected from the group consisting of a blood cell fraction, plasma, and serum. Even more preferably, the blood cell fraction is a fraction of/comprising erythrocytes, leukocytes, and/or thrombocytes.

It is preferred that the lung cancer is lung cancer of stage I or II (i.e. early-stage lung cancer). It is further preferred that the lung cancer is small-cell lung carcinoma (SCLC) or non-small-cell lung carcinoma (NSCLC), particularly lung adenocarcinoma (LUAD) or lung squamous cell carcinoma (LUSC) e.g. small-cell lung carcinoma (SCLC) or non-small-cell lung carcinoma (NSCLC), particularly lung adenocarcinoma (LUAD) or lung squamous cell carcinoma (LUSC), of stage I or II.

As to other preferred embodiments, it is referred to the first to third aspect of the present invention.

More specifically, the kit comprises means for carrying out next generation sequencing (NGS), one or more polynucleotides (probes) for detecting the RNA molecule(s), one or more primers (e.g. primer pairs) for binding the RNA molecule(s), and/or one or more antibodies capable of binding a hybrid of said polynucleotide(s) (probe(s)) and said RNA molecule(s).

The one or more polynucleotide(s) (probe(s)) may be part of a microarray/biochip or may be attached to beads of a beads-based multiplex system. The one or more polynucleotide(s) (primers, primer pairs) may be part of a RT-PCR system, a PCR-system, or a next generation sequencing system.

Said means may further comprise a microarray, a RT-PCT system, a PCR-system, a flow cytometer, a Luminex system, and/or a next generation sequencing system.

In one preferred embodiment, the kit is useful for conducting the methods of the first or second aspect of the present invention.

In one another preferred embodiment, the kit further comprises instructions on how to carry out the methods of the first or second aspect of the present invention.

The kit may further comprise
a container, and/or
a data carrier.

The data carrier may be a non-electronical data carrier, e.g. a graphical data carrier such as an information leaflet, an information sheet, a bar code or an access code, or an electronical data carrier such as a floppy disk, a compact disk (CD), a digital versatile disk (DVD), a microchip or another semiconductor-based electronical data carrier. The access code may allow the access to a database, e.g. an internet database, a centralized, or a decentralized database. The access code may also allow access to an application software that causes a computer to perform tasks for computer users or a mobile app which is a software designed to run on smartphones and other mobile devices.

Said data carrier may further comprise at least one reference, e.g. the reference level(s) of the level(s) of the RNA molecule(s) determined herein and/or the cut-off score(s). In case that the data carrier comprises an access-code which allows the access to a database, said at least one reference, e.g. reference level(s) of the level(s) of the RNA molecule(s) determined herein and/or the cut-off score(s), may be deposited in this database.

The data carrier may also comprise information or instructions on how to carry out the methods of the first or second aspect of the present invention.

The kit may also comprise materials desirable from a commercial and user standpoint including a buffer(s), a reagent(s) and/or a diluent(s) for determining the level mentioned above.

In a further aspect, the present relates invention to a method of determining/distinguishing/differentiating whether a patient is healthy or suffers from a subtype of lung cancer, in particularly suffers from small-cell lung carcinoma (SCLC), suffers from lung adenocarcinoma (LUAD), or suffers from lung squamous cell carcinoma (LUSC), comprising the step of:
determining the levels of at least two RNA molecules in a blood sample from a patient, wherein the at least two RNA molecules comprise a first RNA molecule and a second RNA molecule, wherein the first RNA molecule comprises a nucleotide sequence according to SEQ ID NO: 2, is a fragment thereof, or has a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and the second RNA molecule comprises a nucleotide sequence according to SEQ ID NO: 3, is a fragment thereof, or has a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

In one preferred embodiment, the level of the RNA molecule comprising a nucleotide sequence according to SEQ ID NO: 2, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto, the level of the RNA molecule comprising a nucleotide sequence according to SEQ ID NO: 3, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto, and the level of at least one further RNA molecule selected from the group consisting of an RNA molecule comprising a nucleotide sequence according to SEQ ID NO: 1, SEQ ID NO: 4 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are determined in the blood sample from the patient.

Preferably, the levels of the RNA molecules determined above are (subsequently) used/considered in a mathematical computation to obtain a numerical value (z).

More preferably, the numerical value is obtained by summarizing the weighted levels of the at least two RNA molecules.

Even more preferably, the numerical value (z) is (afterwards) compared to an empirically determined cut-off score for healthiness or (a) subtype(s) of lung cancer, particularly lung adenocarcinoma (LUAD), lung squamous cell carcinoma (LUSC), and/or small-cell lung carcinoma (SCLC). This comparison allows to determine whether the patient is healthy or suffers from a subtype of lung cancer, particularly LUAD, LUSC, or SCLC.

In one more preferred embodiment,
(i) the levels of the RNA molecules comprising a nucleotide sequence according to SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, and SEQ ID NO: 79 are determined in the blood sample from the patient,
(ii) the levels of the RNA molecules determined in step (i) are used/considered in a mathematical computation to obtain a numerical value (z), and
(iii) the numerical value (z) is compared to an empirically determined cut-off score for healthiness, Adenocarcinoma (LUAD), squamous cell carcinoma (LUSC), and/or small-cell lung carcinoma (SCLC), wherein a numerical value (z) which is below the cut-off score for LUAD indicates that the patient is healthy (not suffering from lung cancer), a numerical value (z) which is above the cut-off score for healthiness and below the cut-off score for LUSC indicates that the patient suffers from LUAD, a numerical value (z) which is below the cut-off score for SCLC and above the cut-off score for LUAD indicates that the patient suffers from LUSC, or a numerical value (z) which is above the cut-off score for LUSC indicates that the patient suffers from SCLC.

Preferably, the numerical value is obtained by summarizing the weighted levels of the at least two RNA molecules.

As mentioned above, the present invention relates, in a further aspect, to a method of determining/distinguishing/differentiating whether a patient is healthy or suffers from a subtype of lung cancer, particularly suffers from small-cell lung carcinoma (SCLC), suffers from lung adenocarcinoma (LUAD), or suffers from lung squamous cell carcinoma (LUSC), comprising the step of:
determining the levels of at least two RNA molecules in a blood sample from a patient, wherein the at least two RNA molecules comprise a first RNA molecule and a second RNA molecule, wherein the first RNA molecule comprises a nucleotide sequence according to SEQ ID NO: 2, is a fragment thereof, or has a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto and the second RNA molecule comprises a nucleotide sequence according to SEQ ID NO: 3, is a fragment thereof, or has a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto.

In one preferred embodiment,
the level of the RNA molecule comprising a nucleotide sequence according to SEQ ID NO: 2, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto, the level of the RNA molecule comprising a nucleotide sequence according to SEQ ID NO: 3, a fragment thereof, or a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto, and
the level of at least one further RNA molecule selected from the group consisting of an RNA molecule comprising a nucleotide sequence according to SEQ ID NO: 1, SEQ ID NO: 4 to SEQ ID NO: 79, a fragment thereof, and a sequence having at least 80%, preferably at least 85%, more preferably at least 90%, and most preferably at least 95% or 99%, e.g. at least 80, 81, 82, 83, 84, 85, 86, 87, 88, 89, 90, 91, 92, 93, 94, 95, 96, 97, 98, or 99%, sequence identity thereto are determined in the blood sample from the patient.

In one alternative more preferred embodiment,
(i) the levels of the RNA molecules comprising a nucleotide sequence according to SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, and SEQ ID NO: 79 are determined in the blood sample from the patient, and
(ii) the levels of the RNA molecules are used/considered in a mathematical computation to obtain numerical values z1 to z4 according to the following formulas:

$$z 1 = f ( - 0.639 * x 2 + - 0.479 * x 3 + - 0.243 * x 7 + 0.264 * x 11 + 0.094 * x 14 + 0.252 * x 17 + - 0.271 * x 18 + 0.391 * x 21 + 0.53 * x 28 + - 0.215 * x 56 + - 0.189 * x 72 + - 0.313 * x 73 + 0. * x 74 + 0.196 * x 75 + 0.181 * x 76 + - 0.075 * x 77 + - 0.071 * x 78 + 0.000 * x 79 + - 1.823 ) ,$$

$$f ( x ) = 1 \underline{/} ( 1 + exp ( - x ) ) ) ) ,$$

wherein
x2, x3, x7, x11, x14, x17, x18, x21, x28, x56, x72, x73, x74, x75, x76, x77, x78, x79 is the log 2-RPM expression of SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, respectively, and RPM stands for reads per million,

$$z 2 = f ( 0.142 * x 2 + 0.272 * x 3 + - 0.207 * x 7 + 0. * x 11 + - 0.143 * x 14 + - 0.134 * x 17 + 0.008 * x 18 + - 0.075 * x 21 + 0.124 * x 28 + - 0.023 * x 56 + 0.287 * x 72 + 0.085 * x 73 + - 0.448 * x 74 + 0. * x 75 + - 0. * x 76 + 0.076 * x 77 + - 0.126 * x 78 + - 0.017 * x 79 + - 0.712 ) ,$$

$$f ( x ) = 1 \underline{/} ( 1 + exp ( - x ) ) ) ) ,$$

wherein
x2, x3, x7, x11, x14, x17, x18, x21, x28, x56, x72, x73, x74, x75, x76, x77, x78, x79 is the log 2-RPM expression of SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, respectively, and RPM stands for reads per million.

$$z 3 = f ( - 0.306 * x 2 + - 0.088 * x 3 + 1.368 * x 7 + 0.095 * x 11 + - 0.294 * x 14 + 0.140 * x 17 + 0.182 * x 18 + 0.002 * x21 + - 0.099 * x 28 + 0.085 * x 56 + 0.229 * x72 + - 0.216 * x 73 + 0.064 * x 74 + - 0.527 * x 75 + 0.066 * x 76 + 0.038 * x 77 + 0.002 * x 78 + - 0.053 * x 79 + 1.186 ) ,$$

$$f ( x ) = 1 \underline{/} ( 1 + exp ( - x ) ) ) ) ,$$

wherein
x2, x3, x7, x11, x14, x17, x18, x21, x28, x56, x72, x73, x74, x75, x76, x77, x78, x79 is the log 2-RPM expression of SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, respectively, and RPM stands for reads per million, and

$$z 4 = f ( 0.803 * x 2 + 0.287 * x 3 + - 0.587 * x 7 + 0.029 * x 11 + 0.344 * x 14 + - 0.257 * x 17 + 0. * x 18 + - 0.287 * x 21 + - 0.553 * x 28 + 0.154 * x 56 + - 0.327 * x 72 + 0.442 * x 73 + 0.059 * x 74 + 0.009 * x 75 + - 0.257 * x 76 + - 0.045 * x 77 + 0.457 * x 78 + 0.131 * x 79 + 1.35 ) ,$$

$$f ( x ) = 1 \underline{/} ( 1 + exp ( - x ) ) ) ) ,$$

wherein
x2, x3, x7, x11, x14, x17, x18, x21, x28, x56, x72, x73, x74, x75, x76, x77, x78, x79 is the log 2-RPM expression of SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, respectively, and RPM stands for reads per million,
wherein
z1>z2 and z1>z3 and z1>z4 indicates that the patient is healthy (does not suffer from any type of lung cancer, particularly lung adenocarcinoma (LUAD), lung squamous cell carcinoma (LUSC), or lung squamous cell carcinoma or (SCLC),
z2>z1 and z2>z3 and z2>z4 indicates that the patient suffers from lung adenocarcinoma (LUAD),
z3>z1 and z3>z2 and z3>z4 indicates that the patient suffers from lung squamous cell carcinoma (LUSC), or
z4>z1 and z4>z2 and z4>z3 indicates that the patient suffers from small-cell lung carcinoma (SCLC).

If any two values of z1, z2, z3, z4 are exactly equal, the test is inconclusive.

As to other embodiments, it is referred to the first aspect of the present invention.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURES

The following Figures are merely illustrative of the present invention and should not be construed to limit the scope of the invention as indicated by the appended claims in any way.

FIG. 1: Shows the ROC curve quantiles over 100 model training replicates of the RNA molecule having a nucleotide sequence of SEQ ID NO: 1. AUC (median): 0.777.

FIG. 2: Shows the ROC curve quantiles over 100 replicates of the RNA molecules having nucleotide sequences of SEQ ID NO: 1 and SEQ ID NO: 2. AUC (median): 0.807.

FIG. 3: Shows the ROC curves of 100 model training replicates of the RNA molecules having nucleotide sequences of SEQ ID NO: 1, SEQ ID NO: 2, SEQ ID NO: 19, SEQ ID NO: 35, and SEQ ID NO: 39, and SEQ ID NO: 48. AUC (median): 0.841.

FIG. 4: Shows the ROC curves of 100 model training replicates of the RNA molecules having nucleotide sequences of SEQ ID NO: 1, SEQ ID NO: 2, SEQ ID NO: 23, SEQ ID NO: 39, and SEQ ID NO: 48. AUC (median): 0.848.

FIG. 5: Shows the ROC curves of 100 model training replicates of the RNA molecules having nucleotide sequences of SEQ ID NO: 1, SEQ ID NO: 2, SEQ ID NO: 6, SEQ ID NO: 7, and SEQ ID NO: 9. AUC (median): 0.821.

FIG. 6: Shows the ROC curves of 100 model training replicates of the RNA molecules having nucleotide sequences of SEQ ID NO: 1, SEQ ID NO: 2, SEQ ID NO: 6, SEQ ID NO: 7, and SEQ ID NO: 11, and SEQ ID NO: 18. AUC (median): 0.844.

FIG. 7: Shows the ROC curves of 100 model training replicates of the RNA molecules having nucleotide sequences of SEQ ID NO: 1, SEQ ID NO: 2, SEQ ID NO: 19, SEQ ID NO: 35, and SEQ ID NO: 38, SEQ ID NO: 39, SEQ ID NO: 40, SEQ ID NO: 43, SEQ ID NO: 48, and SEQ ID NO: 49. AUC (median): 0.841.

FIG. 8: Shows the ROC curve quantiles over 100 model training replicates of the RNA molecules having nucleotide sequences of SEQ ID NO: 1, SEQ ID NO: 2, SEQ ID NO: 17, SEQ ID NO: 23, and SEQ ID NO: 35, SEQ ID NO: 39, SEQ ID NO: 45, SEQ ID NO: 48, SEQ ID NO: 52, and SEQ ID NO: 53. AUC (median): 0.857.

FIG. 9: Shows the ROC curve quantiles over 100 model training replicates of the RNA molecules having nucleotide sequences of SEQ ID NO: 1, SEQ ID NO: 2, SEQ ID NO: 6, SEQ ID NO: 7, SEQ ID NO: 9, SEQ ID NO: 19, SEQ ID NO: 25, SEQ ID NO: 29, SEQ ID NO: 30, and SEQ ID NO: 38. AUC (median): 0.839.

FIG. 10: Shows the ROC curve quantiles over 100 model training replicates of the RNA molecules having nucleotide sequences of SEQ ID NO: 1, SEQ ID NO: 2, SEQ ID NO: 3, SEQ ID NO: 6, SEQ ID NO: 7, SEQ ID NO: 11, SEQ ID NO: 12, SEQ ID NO: 14, SEQ ID NO: 18, and SEQ ID NO: 28. AUC (median): 0.871.

FIG. 11: Shows the ROC curve quantiles over 100 replicates of the RNA molecules having nucleotide sequences of SEQ ID NO: 1 to SEQ ID NO: 54. AUC (median): 0.880.

FIG. 12: Shows the ROC curve quantiles over 100 replicates of the RNA molecules having nucleotide sequences of SEQ ID NO: 1, SEQ ID NO: 2, SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, and SEQ ID NO: 71. AUC (median): 0.866.

FIG. 13: Shows the result of the comparison of healthy (control) subjects and patients known to suffer from lung cancer of the signature of 54 RNA molecules. Mean model weight from 100 training replicates, log 2 fold change (DESeq2) and adjusted p-value (DESeq2) are indicated. “0” stands for an asymptotically small value.

FIG. 14: Shows the result of the comparison of healthy (control) subjects and patients known to suffer from lung cancer of the signature of 38 RNA molecules. Mean model weight from 100 training replicates, log 2 fold change (DESeq2) and adjusted p-value (DESeq2) are indicated. “0” stands for an asymptotically small value.

FIG. 15: Shows the result of the comparison of healthy (control) subjects and patients known to suffer from lung cancer of the signature of 18 RNA molecules. Two non-overlapping groups of patients were used, a discovery cohort (498 controls and 445 cancers) and a validation cohort (155 controls and 286 cancers). The logistic regression model was trained on the discovery cohort and ROC curves and AUCs were measured on the validation cohort.

FIG. 16: Shows the result of the comparison of healthy (control) subjects and patients known to suffer from lung cancer of the signature of 6 RNA molecules (Mitra samples). A group of 21 controls and 19 cancers was randomly split into 100 sets of 30 training samples and 10 test samples and separate logistic regression models were trained on all split. A model with median AUC on the test set was used here. Note that blood samples in this specific case were obtained from peripheral blood through finger pricks, not intravenous blood collected in PAXgene.

FIG. 17: Shows the ROC curve with the performance of a model using two RNAs-RNA molecule comprising the nucleotide sequence according to SEQ ID NO: 2 and RNA molecule comprising the nucleotide sequence according to SEQ ID NO: 3—that are part of the miLung18 signature (trained on a large discovery cohort of patients) predicting cases vs controls in a held-out validation cohort. The AUC is 0.774.

FIG. 18: Shows the ROC curve with the performance of the miLung18 model (trained on a large discovery cohort of patients) predicting cases vs controls in a held-out validation cohort. The AUC is 0.832.

FIG. 19: Shows the ROC curve with the performance of the miLung6 model over 100 cross-validation splits of 20 Control and 20 Cancer patients for which blood was obtain using the Mitra finger prick device. The median AUC was 0.735.

FIG. 20: Shows the ROC curve with the performance of a different miLung18 model trained to predict controls and three major sub-types of lung cancer (NSCLC Adenocarcinoma (LUAD), NSCLC Squamous carcinoma (LUSC), SCLC). Each curve corresponds to a different one-vs-rest comparison. The AUC for Control-vs-rest is 0.83, for NSCLC_Adeno-vs-rest is 0.74, for NSCLC_Squamous-vs-rest is 0.87, and for SCLC-vs-rest is 0.92. The model was trained on the discovery cohort, metrics are shown for the held-out validation cohort.

FIG. 21: Scores from the miLung18 model calculated on the validation cohort which was not used for training. Separate bars are shown for controls and different tumor stages, as well as different lung cancer sub-types. A score below 0.5 indicates that the patient was predicted to not have lung cancer, whereas a score above 0.5 indicates a lung cancer prediction.

FIG. 22: Model weights of the lung cancer sub-type prediction model based on the miLung18 RNS signature. Performance of this model shown in FIG. 20.

### EXAMPLES

The examples given below are for illustrative purposes only and do not limit the invention described above in any way.

### Example 1

### Materials

Patients with or without lung cancer (all possible lung cancer types) were included from three Hummingbird sponsored studies (Boston (NCT03452514), Lungenklinik Grosshansdorf (DRKS00018784), Ruhrlandklinik Essen (DRKS00020137). Briefly, blood was collected into PAXgene Blood RNA tubes as per manufacturer instructions and after 10× inversion stored at −20° C. before long storage at −80° C.

### Methods

The RNA from PAXgene samples was extracted using the QIAsymphony PAXgene Blood RNA Kit on the QIAsymphony SP liquid handling station. Eluted RNA was aliquoted and stored at −80° C. 100 ng of total RNA were used for small RNA library preparation. PCR libraries products following 18 cycles of amplification were assessed for size and uniformity and the concentration of the PCR products was determined. NGS libraries were measured on Illumina NextSeq 200 instrument. Small non-coding RNAs were measured from Next Generation Sequencing data, which measures the relative abundance (expression) of a large number of RNAs. Small subsets of RNAs that can be used for the detection of Lung Cancer were identified.

Illumina NextSeq 2000-provided FastQ files from repeated measurements of the same multiplexing pool (batch of samples sequenced simultaneously using dual indexing) were first aggregated to single FastQ files per pool. These were then UMI-corrected (to eliminate PCR-artefacts) and converted into raw read count matrices, summing the UMI-corrected read counts of small RNAs X. Finally, RNA expression was provided as

$${Y}_{ij} = {log}_{2} ( 1 + \frac{{cX}_{ij}}{{M}_{j}} ) ,$$

which are the log 2-transformed, 1-shifted, library size Mⱼ normalized, reads per million (RPM) cXᵢⱼ/Mⱼ, with c=1e6 for every RNA i and patient sample j.

ROC curves were used to quantify the ability of a logistic regression model (with optimized L1 regularization) to distinguish patients with cancer from those without cancer (controls) based on RNA expression.

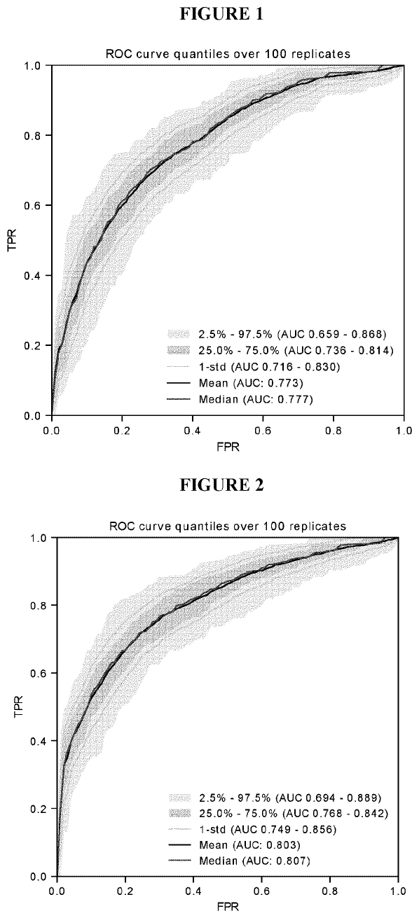

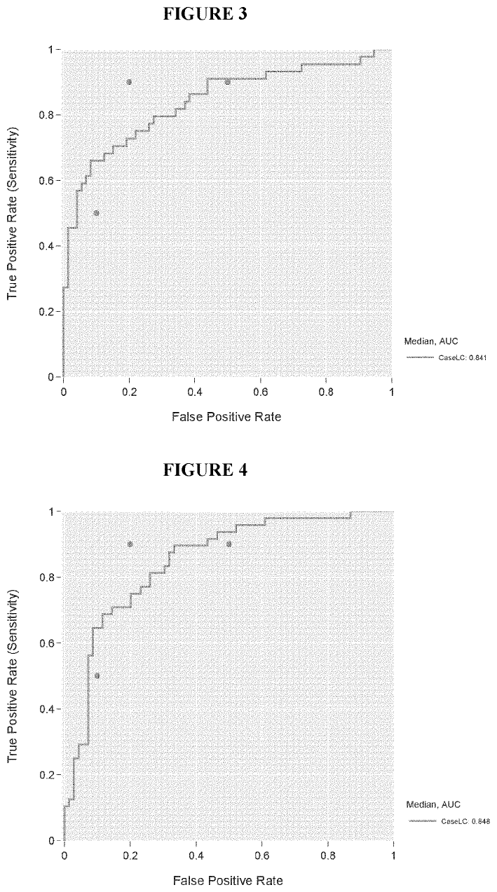

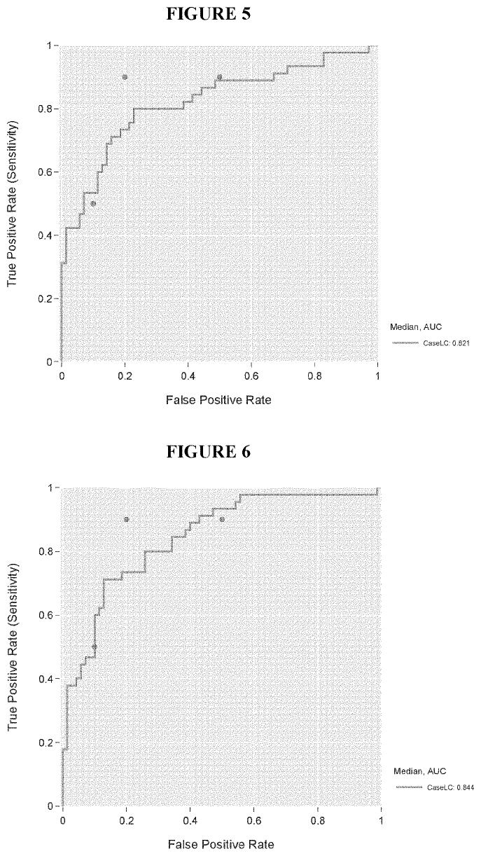

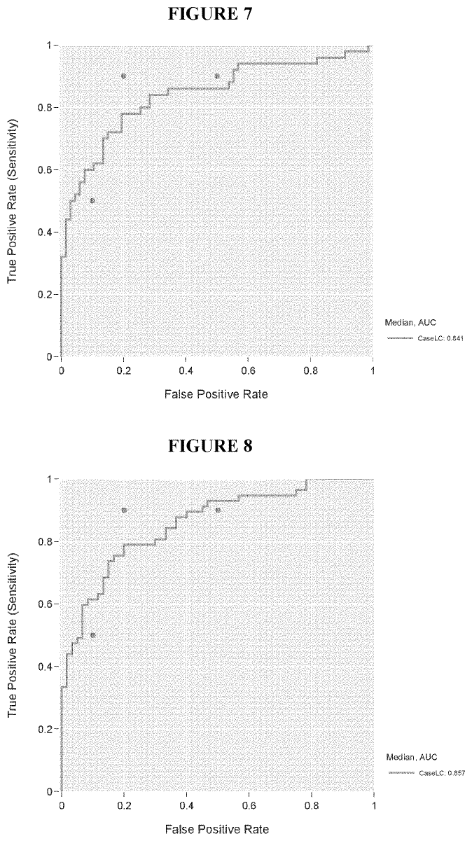

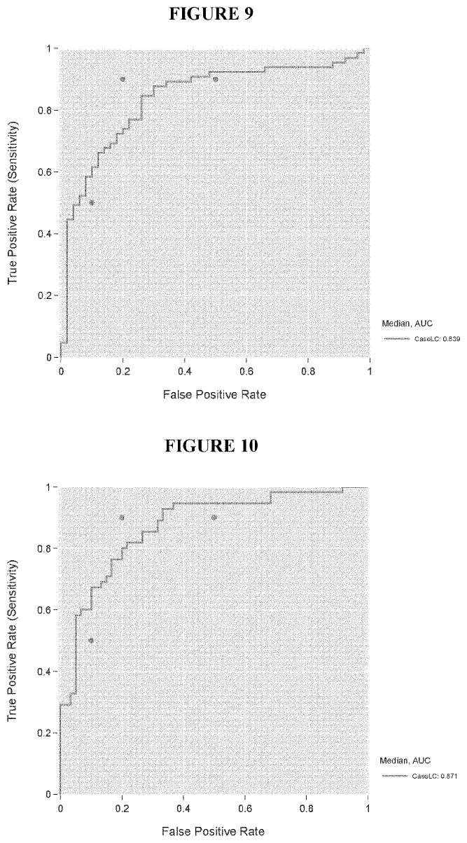

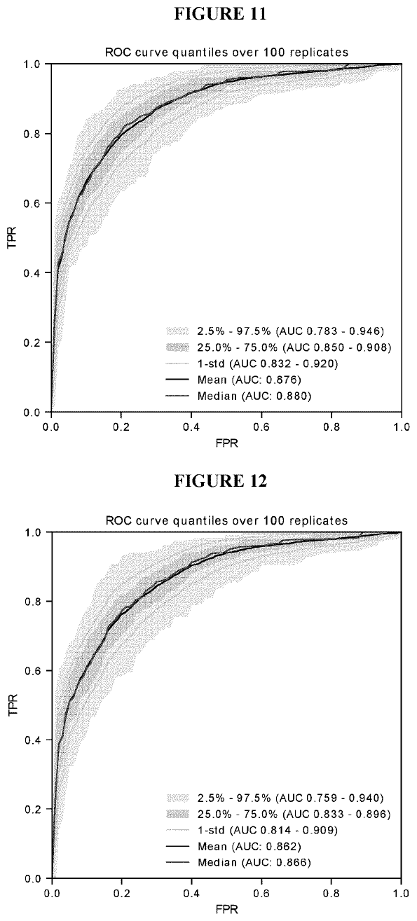

FIGS. 1 to 12 show summary plots of 100 ROC curves, each obtained from a separate split of the patient samples into training and test sets. A separate logistic regression model was trained on each training set and the ROC statistics were obtained on the test set. Summary AUC metrics are given in each plot to specify the expected AUC and its variability across the 100 replicates.

All features were normalized by subtracting the mean and dividing by the standard deviation of training samples.

Already the RNA molecule having a nucleotide sequence according to SEQ ID NO: 1 has a median AUC value of 0.77 and is, thus, of high diagnostic relevance for the detection of lung cancer in a patient. The ROC curve of this RNA molecule is shown in FIG. 1.

In addition, the following RNA molecule signatures with high diagnostic relevance were calculated:
(i) SEQ ID NO: 1 and SEQ ID NO: 2,
(ii) SEQ ID NO: 1, SEQ ID NO: 2, SEQ ID NO: 19, SEQ ID NO: 35, and SEQ ID NO: 39, and SEQ ID NO: 48,
(iii) SEQ ID NO: 1, SEQ ID NO: 2, SEQ ID NO: 23, SEQ ID NO: 39, and SEQ ID NO: 48,
(iv) SEQ ID NO: 1, SEQ ID NO: 2, SEQ ID NO: 6, SEQ ID NO: 7, and SEQ ID NO: 9,
(v) SEQ ID NO: 1, SEQ ID NO: 2, SEQ ID NO: 6, SEQ ID NO: 7, and SEQ ID NO: 11, and SEQ ID NO: 18,
(vi) SEQ ID NO: 1, SEQ ID NO: 2, SEQ ID NO: 19, SEQ ID NO: 35, and SEQ ID NO: 38, SEQ ID NO: 39, SEQ ID NO: 40, SEQ ID NO: 43, SEQ ID NO: 48, and SEQ ID NO: 49, SEQ ID NO: 1, SEQ ID NO: 2, SEQ ID NO: 17, SEQ ID NO: 23, and SEQ ID NO: 35, (vii) SEQ ID NO: 39, SEQ ID NO: 45, SEQ ID NO: 48, SEQ ID NO: 52, and SEQ ID NO: 53,
(viii) SEQ ID NO: 1, SEQ ID NO: 2, SEQ ID NO: 6, SEQ ID NO: 7, SEQ ID NO: 9, SEQ ID NO: 19, SEQ ID NO: 25, SEQ ID NO: 29, SEQ ID NO: 30, and SEQ ID NO: 38,
(ix) SEQ ID NO: 1, SEQ ID NO: 2, SEQ ID NO: 3, SEQ ID NO: 6, SEQ ID NO: 7, SEQ ID NO: 11, SEQ ID NO: 12, SEQ ID NO: 14, SEQ ID NO: 18, and SEQ ID NO: 28, SEQ ID NO: 1 to SEQ ID NO: 54, and (x)
(xi) SEQ ID NO: 1, SEQ ID NO: 2, SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, and SEQ ID NO: 71.

The ROC curves of the above RNA molecule signatures are shown in FIGS. 2 to 12. AUC values >0.8 could be achieved with a combination of RNA molecules (RNA signatures).

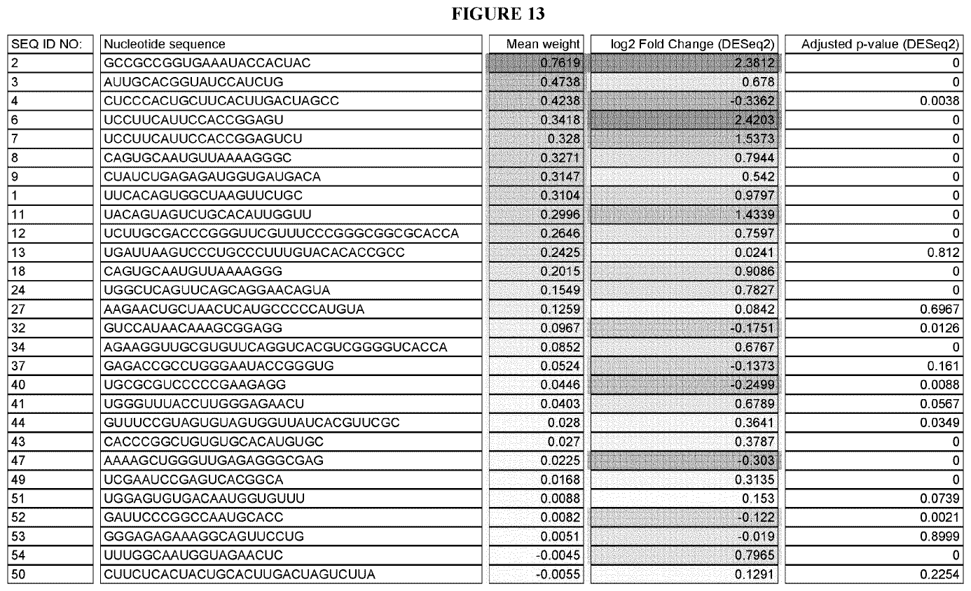

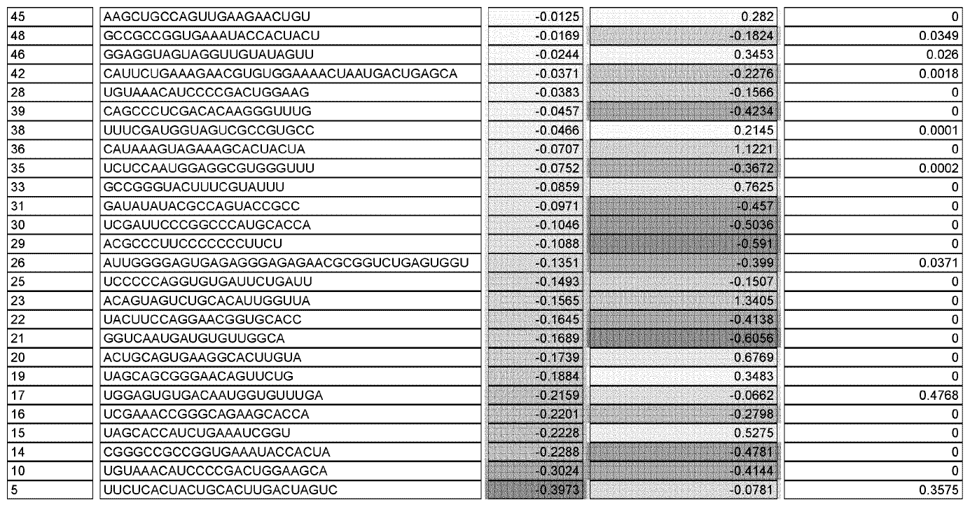

The result of the comparison of healthy (control) subjects and patients known to suffer from lung cancer of the signature of 54 RNA molecules, including Mean model weight from 100 training replicates, log 2 fold change (DESeq2) and adjusted p-value (DESeq2) of the single RNA molecules, are shown in FIG. 13.

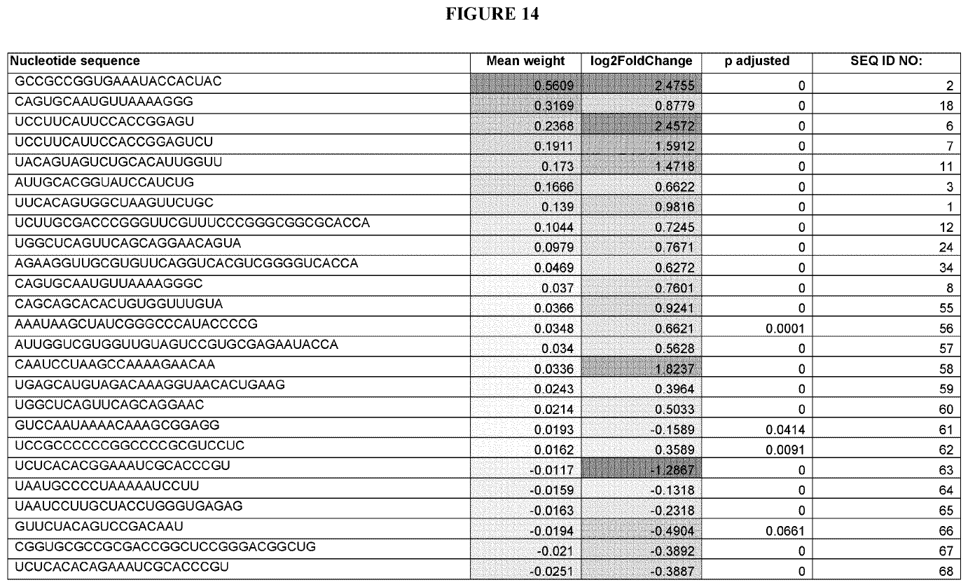

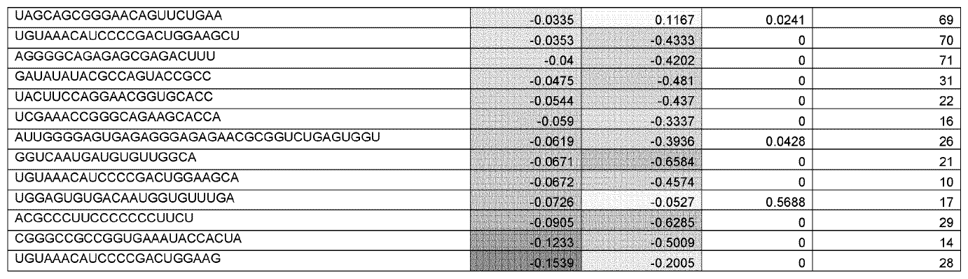

The result of the comparison of healthy (control) subjects and patients known to suffer from lung cancer of the signature of 38 RNA molecules, including Mean model weight from 100 training replicates, log 2 fold change (DESeq2) and adjusted p-value (DESeq2) of the single RNA molecules are shown in FIG. 14.

All subsets of the maximal signature that also contain the minimal signature are expected to have a performance between that of the minimal and the maximal signature, i.e. all of these subsets can also be used for the detection of lung cancer.

To allow the classification of patients as patients suffering from lung cancer or as patients not suffering from lung cancer, i.e. being healthy, the following formulas were calculated (with adjusted median weights) for the different signatures:
(i) SEQ ID NO: 1 and SEQ ID NO: 2:

$$z = f ( 0.95 * x 1 + 0.71 * x 2 + - 5.71 ) ,$$

$$f ( x ) = 1 / ( 1 + exp ( - x ) ) ) ) ,$$

wherein
x1 and x2 is the standardized log 2-RPM expression of SEQ ID NO: 1 and SEQ ID NO: 2, and
RPM stands for reads per million,
(ii) SEQ ID NO: 1, SEQ ID NO: 2, SEQ ID NO: 19, SEQ ID NO: 35, SEQ ID NO: 39, and SEQ ID NO: 48:

$$z = f ( 0.94 * x 1 + 0.86 * x 2 + - 0.07 * x 19 + - 0.33 * x 35 + - 0.15 * x 39 + - 0.33 * x 48 + - 3.86 ) ,$$

$$f ( x ) = 1 / ( 1 + exp ( - x ) ) ) ) ,$$

wherein
x1, x2, x19, x35, x39, and x48 is the standardized log 2-RPM expression of SEQ ID NO: 1, SEQ ID NO: 2, SEQ ID NO: 19, SEQ ID NO: 35, SEQ ID NO: 39, and SEQ ID NO: 48, respectively, and
RPM stands for reads per million,
(iii) SEQ ID NO: 1, SEQ ID NO: 2, SEQ ID NO: 23, SEQ ID NO: 39, and SEQ ID NO: 48:

$$z = f ( 0.44 * x 1 + 0.83 * x 2 + 0.45 * x 23 + - 0.07 * x 39 + - 0.25 * x 48 + - 4.34 ) ,$$

$$f ( x ) = 1 / ( 1 + exp ( - x ) ) ) ) ,$$

wherein
x1, x2, x23, x39, and x48 is the standardized log 2-RPM expression of SEQ ID NO: 1, SEQ ID NO: 2, SEQ ID NO: 23, SEQ ID NO: 39, and SEQ ID NO: 48, respectively, and RPM stands for reads per million,
(iv) SEQ ID NO: 1, SEQ ID NO: 2, SEQ ID NO: 6, SEQ ID NO: 7, and SEQ ID NO: 9:

$$z = f ( 0.84 * x 1 + 0.6 * x 2 + 0.85 * x 6 + 0.23 * x 7 + 0.3 * x 9 + - 6.44 ) ,$$

$$f ( x ) = 1 / ( 1 + exp ( - x ) ) ) ) ,$$

wherein
x1, x2, x6, x7, and x9 is the standardized log 2-RPM expression of SEQ ID NO: 1, SEQ ID NO: 2, SEQ ID NO: 6, SEQ ID NO: 7, SEQ ID NO: 9, respectively, and RPM stands for reads per million,
(v) SEQ ID NO: 1, SEQ ID NO: 2, SEQ ID NO: 6, SEQ ID NO: 7, and SEQ ID NO: 11, and SEQ ID NO: 18:

$$z = f ( 0.19 * x 1 + 0.62 * x 2 + 0.87 * x 6 + 0.32 * x 7 + 0.4 * x 11 + 0.65 * x 18 + - 6.12 ) ,$$

$$f ( x ) = 1 / ( 1 + exp ( - x ) ) ) ) ,$$

wherein
x1, x2, x6, x7, x11, and x18 is the standardized log 2-RPM expression of SEQ ID NO: 1, SEQ ID NO: 2, SEQ ID NO: 6, SEQ ID NO: 7, SEQ ID NO: 11, and SEQ ID NO: 18, respectively, and
RPM stands for reads per million,
(vi) SEQ ID NO: 1, SEQ ID NO: 2, SEQ ID NO: 19, SEQ ID NO: 35, SEQ ID NO: 38, SEQ ID NO: 39, SEQ ID NO: 40, SEQ ID NO: 43, SEQ ID NO: 48, and SEQ ID NO: 49:

$$z = f ( 0.93 * x 1 + 0.83 * x 2 + - 0.09 * x 19 + - 0.33 * x 35 + - 0.11 * x 38 + - 0.13 * x 39 + - 0 \text{.12} * x 40 + 0.17 * x 43 + - 0.29 * x 48 + 0.1 * x 49 + - 4.41 ) ,$$

$$f ( x ) = 1 / ( 1 + exp ( - x ) ) ) ) ,$$

wherein
x1, x2, x19, x35, x38, x39, x40, x43, x48, and x49 is the standardized log 2-RPM expression of SEQ ID NO: 1, SEQ ID NO: 2, SEQ ID NO: 19, SEQ ID NO: 35, SEQ ID NO: 38, SEQ ID NO: 39, SEQ ID NO: 40, SEQ ID NO: 43, SEQ ID NO: 48, and SEQ ID NO: 49, respectively, and
RPM stands for reads per million,
(vii) SEQ ID NO: 1, SEQ ID NO: 2, SEQ ID NO: 17, SEQ ID NO: 23, SEQ ID NO: 35, SEQ ID NO: 39, SEQ ID NO: 45, SEQ ID NO: 48, SEQ ID NO: 52, and SEQ ID NO: 53:

$$z = f ( 0.52 * x 1 + 0.93 * x 2 + - 0.22 * x 17 + 0.43 * x 23 + - 0.22 * x 35 + - 0.08 * x 39 + - 0 \text{.06} * x 45 + - 0.22 * x 48 + - 0.32 * x 52 + 0.02 * x 53 + - 1.32 ) ,$$

$$f ( x ) = 1 / ( 1 + exp ( - x ) ) ) ) ,$$

wherein
x1, x2, x17, x23, x35, x39, x45, x48, x52, and x53 is the standardized log 2-RPM expression of SEQ ID NO: 1, SEQ ID NO: 2, SEQ ID NO: 17, SEQ ID NO: 23, SEQ ID NO: 35, SEQ ID NO: 39, SEQ ID NO: 45, SEQ ID NO: 48, SEQ ID NO: 52, and SEQ ID NO: 53, respectively, and
RPM stands for reads per million,
(viii) SEQ ID NO: 1, SEQ ID NO: 2, SEQ ID NO: 6, SEQ ID NO: 7, SEQ ID NO: 9, SEQ ID NO: 19, SEQ ID NO: 25, SEQ ID NO: 29, SEQ ID NO: 30, and SEQ ID NO: 38:

$$z = f ( 0.83 * x 1 + 0.77 * x 2 + 0.69 * x 6 + 0.31 * x 7 + 0.34 * x 9 + - 0.03 * x 19 + - 0.58 * x 25 + - 0.33 * x 29 + - 0.28 * x 30 + - 0.19 * x 38 + 3.17 ) ,$$

$$f ( x ) = 1 / ( 1 + exp ( - x ) ) ) ) ,$$

wherein
x1, x2, x6, x7, x9, x19, x25, x29, x30, and x38 is the standardized log 2-RPM expression of SEQ ID NO: 1, SEQ ID NO: 2, SEQ ID NO: 6, SEQ ID NO: 7, SEQ ID NO: 9, SEQ ID NO: 19, SEQ ID NO: 25, SEQ ID NO: 29, SEQ ID NO: 30, and SEQ ID NO: 38, respectively, and
RPM stands for reads per million,
(ix) SEQ ID NO: 1, SEQ ID NO: 2, SEQ ID NO: 3, SEQ ID NO: 6, SEQ ID NO: 7, SEQ ID NO: 11, SEQ ID NO: 12, SEQ ID NO: 14, SEQ ID NO: 18, and SEQ ID NO: 28:

$$z = f ( 0.24 * x 1 + 0.74 * x 2 + 0.19 * x 3 + 0.68 * x 6 + 0.36 * x 7 + 0.26 * x 11 + 0.26 * x 12 + - 0.42 * x 14 + 0.66 * x 18 + - 0.94 * x 28 + 4.76 ) ,$$

$$f ( x ) = 1 / ( 1 + exp ( - x ) ) ) ) ,$$

wherein
x1, x2, x3, x6, x7, x11, x12, x14, x18, and x28 is the standardized log 2-RPM expression of SEQ ID NO: 1, SEQ ID NO: 2, SEQ ID NO: 3, SEQ ID NO: 6, SEQ ID NO: 7, SEQ ID NO: 11, SEQ ID NO: 12, SEQ ID NO: 14, SEQ ID NO: 18, and SEQ ID NO: 28, respectively, and
RPM stands for reads per million,
(x) SEQ ID NO: 1 to SEQ ID NO: 54:

$$z = f ( 0.45 * x 1 + 0.83 * x 2 + 0.74 * x 3 + 0.5 * x 4 + - 0.48 * x 5 + 0.74 * x 6 + 0.48 * x 7 + 0.55 * x 8 + 0.64 * x 9 + - 0.72 * x 10 + 0.39 * x 11 + 0.25 * x 12 + 0.35 * x 13 + - 0.34 * x 14 + - 0.43 * x 15 + - 0.4 * x 16 + - 0.34 * x 17 + 0.14 * x 18 + - 0.32 * x 19 + - 0.52 * x 20 + - 0.28 * x 21 + - 0.32 * x 22 + - 0.28 * x 23 + 0.2 * x 24 + - 0.33 * x 25 + - 0.24 * x 26 + 0.26 * x 27 + 0.44 * x 28 + - 0.11 * x 29 + - 0.12 * x 30 + - 0.1 * x 31 + 0.25 * x 32 + - 0.26 * x 33 + 0.16 * x 34 + - 0.15 * x 35 + - 0.24 * x 36 + 0.1 * x 37 + - 0.11 * x 38 + - 0.08 * x 39 + 0.08 * x 40 + 0.05 * x 41 + - 0.1 * x 42 + 0.06 * x 43 + 0.06 * x 44 + 0.12 * x 45 + - 0.06 * x 46 + 0.1 * x 47 + - 0.02 * x 48 + 0.01 * x 49 + - 0.04 * x 50 + 0.02 * x 51 + 0.01 * x 52 + 0.04 * x 53 + 0.05 * x 54 + 0.21 ) ,$$

$$f ( x ) = 1 / ( 1 + exp ( - x ) ) ) ) ,$$

wherein x1 to x54 is the standardized log 2-RPM expression of SEQ ID NO: 1 to SEQ ID NO: 54, respectively, and
RPM stands for reads per million, or
(xi) SEQ ID NO: 1, SEQ ID NO: 2, SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, and SEQ ID NO: 71:

$$z = f ( 0.05 * x 1 + 0.83 * x 2 + 0.27 * x 3 + 0.89 * x 6 + 0.35 * x 7 + 0.29 * x 8 + - 0.56 * x 10 + 0.14 * x 11 + 0.13 * x 12 + - 0.16 * x 14 + - 0.21 * x 16 + - 0.29 * x 17 + 0.37 * x 18 + - 0.21 * x 21 + - 0.26 * x 22 + - 0.15 * x 24 + - 0.26 * x 26 + - 0.74 * x 28 + - 0.17 * x 29 + - 0.17 * x 31 + 0.17 * x 34 + 0.16 * x 55 + 0.18 * x 56 + 0.19 * x 57 + 0.08 * x 58 + 0.27 * x 59 + 0.39 * x 60 + 0.5 * x 61 + 0.24 * x 62 + - 0.1 * x 63 + - 0.19 * x 64 + - 0.12 * x 65 + - 0.54 * x 66 + - 0.07 * x 67 + - 0.2 * x 68 + - 0.25 * x 69 + 0.81 * x 70 + - 0.19 * x 71 + 0.33 ) ,$$

$$f ( x ) = 1 / ( 1 + exp ( - x ) ) ) ) ,$$

wherein x1, x2, x3, x6, x7, x8, x10, x11, x12, x14, x16, x17, x18, x21, x22, x24, x26, x28, x29, x31, x34, x55 to x71 is the standardized log 2-RPM expression of SEQ ID NO: 1, SEQ ID NO: 2, SEQ ID NO: 3, SEQ ID NO: 6, SEQ ID NO: 7, SEQ ID NO: 8, SEQ ID NO: 10, SEQ ID NO: 11, SEQ ID NO: 12, SEQ ID NO: 14, SEQ ID NO: 16, SEQ ID NO: 17, SEQ ID NO: 18, SEQ ID NO: 21, SEQ ID NO: 22, SEQ ID NO: 24, SEQ ID NO: 26, SEQ ID NO: 28, SEQ ID NO: 29, SEQ ID NO: 31, SEQ ID NO: 34, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, and SEQ ID NO: 71, respectively, and
RPM stands for reads per million.

In all cases described above, the cut-off score (y) was/was calculated to be 0.5, whereby z>0.5 obtained for the patient indicates that the patient suffers from lung cancer, and z<0.5 obtained for the patient indicates that the patient is healthy (does not suffer from lung cancer).

### Example 2

### Materials

Patients with or without lung cancer (all possible lung cancer types) were included from three Hummingbird sponsored studies (Boston (NCT03452514), Lungenklinik Grosshansdorf (DRKS00018784), Ruhrlandklinik Essen (DRKS00020137). Additional samples were collected from Kempten, Konstanz, and Heidelberg. Briefly, blood was collected into PAXgene Blood RNA tubes as per manufacturer instructions and after 10× inversion stored at −20° C. before long storage at −80° C.

### Methods

The RNA from PAXgene samples was extracted and sequenced as described above.

A discovery cohort was composed of 498 control patients (no cancer) and 445 lung cancer patients from Boston, Essen, and Grosshansdorf sites. A validation cohort was composed of 155 controls and 286 lung cancers from Boston, Kempten, Konstanz, and Heidelberg sites. Logistic Regression models consisting of the listed RNA molecules were trained on the discovery cohort to predict lung cancer vs no lung cancer. Input data was the log 2-RPM expression of these small RNA molecules.

Effect sizes are given as Cohen's d as an alternative to DEseq2 fold change to indicate the difference and directionality of expression between controls and cancers, where a positive d indicates an increase in cancer.

The following RNA molecule signature (2) with high diagnostic relevance was calculated:
SEQ ID NO: 2 and SEQ ID NO: 3:

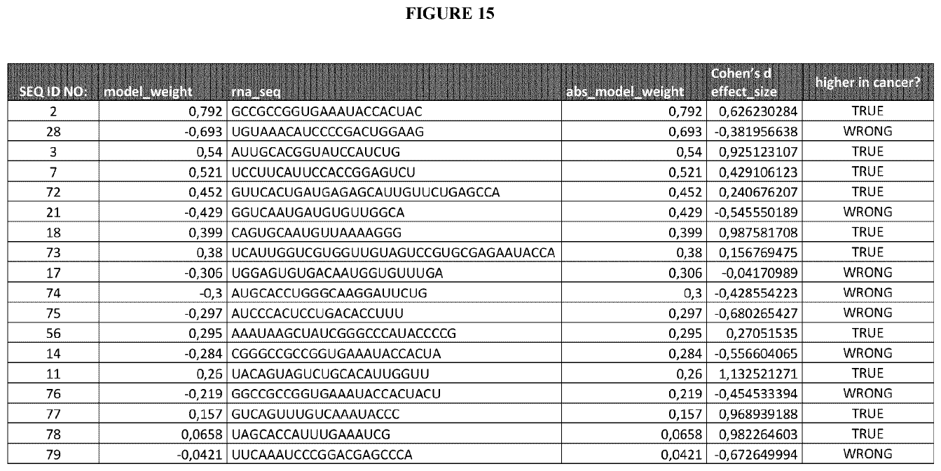

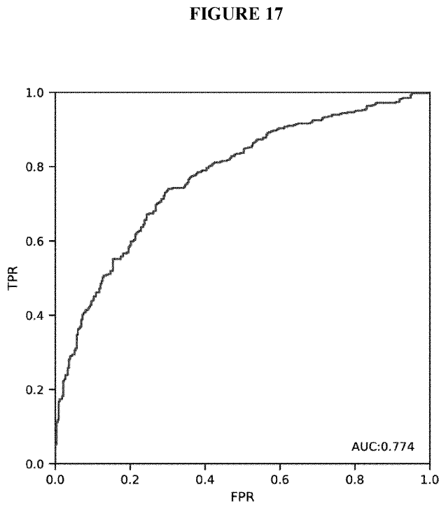

With respect to the data, it is referred to FIG. 15. The ROC curve of this signature is shown in FIG. 17. An AUC value >0.75, namely 0.774, could be achieved.

To allow the classification of patients as patients suffering from lung cancer or as patients not suffering from lung cancer, i.e. being healthy, the following formula was calculated (with adjusted median weights) for this (2) signature:

$$z = f ( 0.8 * x 2 + 1.342 * x 3 + - 8.733 ) ,$$

$$f ( x ) = 1 \underline{/} ( 1 + exp ( - x ) ) ) ) ,$$

wherein x2, x3 is the log 2-RPM expression of SEQ ID NO: 2, SEQ ID NO: 3, respectively, RPM stands for reads per million.

In addition, the following RNA molecule signature (18) with high diagnostic relevance was calculated: SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, and SEQ ID NO: 79.

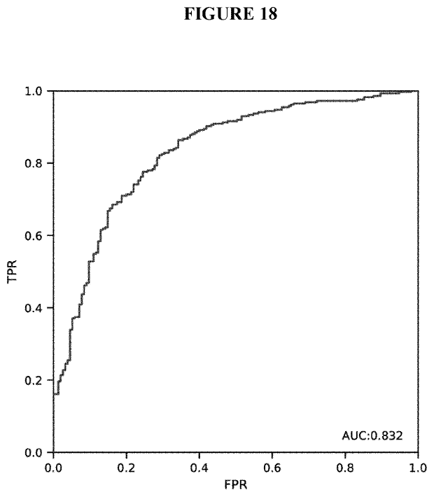

With respect to the data, it is referred to FIG. 15. The ROC curve of this signature is shown in FIG. 18. An AUC value >0.8, namely 0.832, could be achieved.

To allow the classification of patients as patients suffering from lung cancer or as patients not suffering from lung cancer, i.e. being healthy, the following formula was calculated (with adjusted median weights) for this (18) signature:
SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, and SEQ ID NO: 79:

$$z = f ( 0.792 * x 2 + 0.54 * x 3 + 0.521 * x 7 + 0.26 * x 11 + - 0.284 * x 14 + - 0.306 * x 17 + 0.399 * x 18 + - 0.429 * x 21 + - 0.693 * x 28 + 0.295 * x 56 + 0.452 * x 72 + 0.38 * x 73 + - 0.3 * x 74 + - 0.297 * x 75 + - 0.219 * x 76 + 0.157 * x 77 + 0 \text{.0658} * x 78 + - 0.0421 * x 79 + 5.203 ) ,$$

$$f ( x ) = 1 \underline{/} ( 1 + exp ( - x ) ) ) ) ,$$

wherein
x2, x3, x7, x11, x14, x17, x18, x21, x28, x56, x72, x73, x74, x75, x76, x77, x78, x79 is the log 2-RPM expression of SEQ ID NO: 2, SEQ ID NO: 3, SEQ ID NO: 7, SEQ ID NO: 11, SEQ ID NO: 14, SEQ ID NO: 17, SEQ ID NO: 18, SEQ ID NO: 21, SEQ ID NO: 28, SEQ ID NO: 56, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, respectively, and
RPM stands for reads per million.

### Example 3

### Materials & Methods

A clean, disinfected finger was pricked with a lancet. Blood drops were massaged out from the finger. The first drop was discarded. Without touching the skin, 2× Microsampler were applied directly to the blood drop until they soaked up 10 μl each. The cap was then closed, and the Mitra Microsamplers were stored at room temperature. For RNA extraction, Mitra cellulose was transferred into 2 ml Eppendorf tube and 1 ml of QIAzol (QIAgen, Venlo, The Netherlands) was added. The sample was incubated 15 min at room temperature while shaking at 1000 rpm. Next, samples were centrifuged for 5 min with 12,000×g at room temperature and the cellulose was removed. 200 μl Chloroform was added and samples were pulse vortexed for 15 s. After 2 min incubation at RT, samples were centrifuged for 15 min at 12,000×g at 4° C. The upper aqueous phase containing the RNA was mixed with 1.5 volumes of 100% ethanol and further using miRNeasy Serum and Plasma Kit (QIAgen) combined with MinElute spin columns (QIAgen) according to the manufacturer's instructions. The RNA was eluted using 28 μl of nuclease-free water and stored in −80° C. 5-50 ng of total RNA was used for small RNA library preparation as described in Rajakumar et al, 2022 and sequenced on the Illumina NextSeq2000 system.

### DBS Data Analysis

Based on log 2-RPM expression for NGS measurements of small RNAs in 21 controls and 19 cancer patients, 100 logistic regression models were trained, each on a different random 30 samples, while calculating ROC curves and AUC performance on the remaining 10 samples. A representative model with median AUC was picked.

The following RNA molecule signature (6) with high diagnostic relevance was calculated:
SEQ ID NO: 2, SEQ ID NO: 3, SEQ ID NO: 7, SEQ ID NO: 17, SEQ ID NO: 18, and SEQ ID NO: 75.

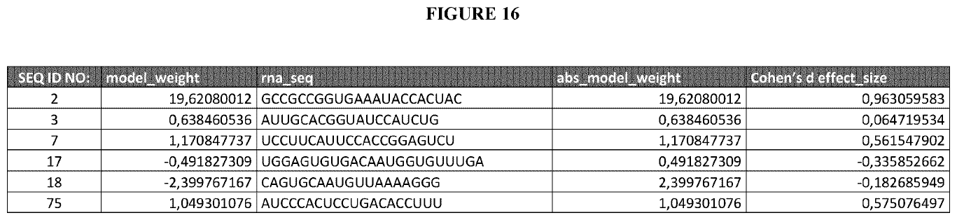

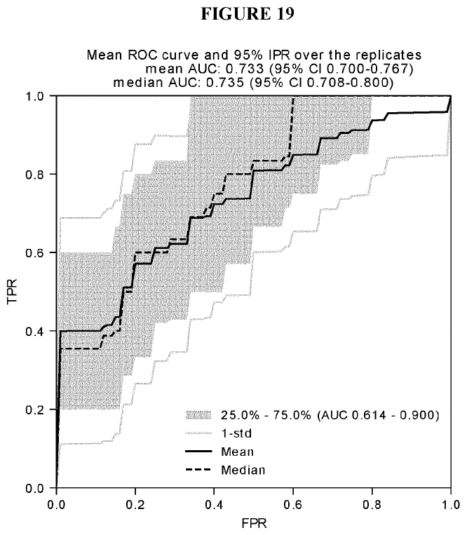

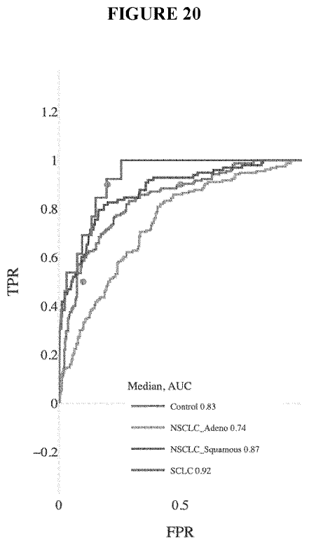

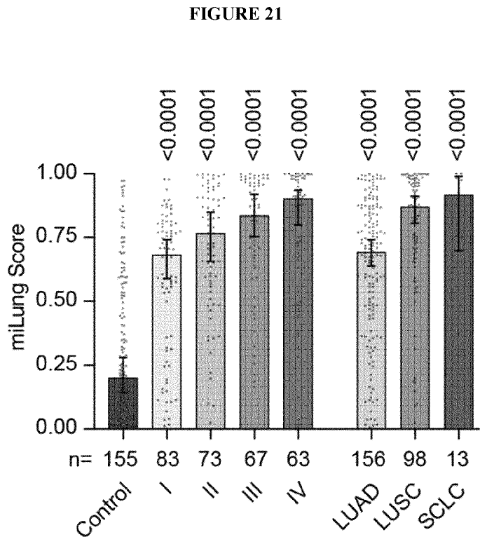

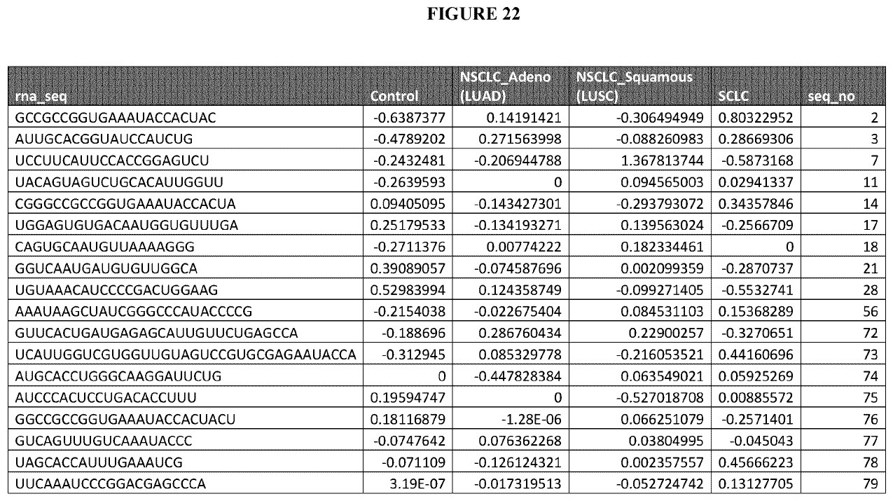

With respect to the data, it is referred to FIG. 16. The ROC curve of this signature is shown in FIG. 19. An AUC value >0.7, namely 0.735, could be achieved.

To allow the classification of patients as patients suffering from lung cancer or as patients not suffering from lung cancer, i.e. being healthy, the following formula was calculated (with adjusted median weights) for this (6) signature:
SEQ ID NO: 2, SEQ ID NO: 3, SEQ ID NO: 7, SEQ ID NO: 17, SEQ ID NO: 18, and SEQ ID NO: 75:

$$z = f ( 19.621 * x 2 + 0.638 * x 3 + 1.171 * x 7 + - 0.492 * x 17 + - 2.4 * x 18 + 1.049 * x 75 + 3.185 ) ,$$

$$f ( x ) = 1 \underline{/} ( 1 + exp ( - x ) ) ) ) ,$$

wherein
x2, x3, x7, x17, x18, x75 is the log 2-RPM expression of SEQ ID NO: 2, SEQ ID NO: 3, SEQ ID NO: 7, SEQ ID NO: 17, SEQ ID NO: 18, SEQ ID NO: 75, respectively, and RPM stands for reads per million.

### Informal Sequence Listing

The RNA molecules having the following sequences were identified as suitable biomarker for the diagnosis and monitoring of lung cancer in a patient:

1.
(SEQ ID NO: 1)
UUCACAGUGGCUAAGUUCUGC
2.
(SEQ ID NO: 2)
GCCGCCGGUGAAAUACCACUAC
3.
(SEQ ID NO: 3)
AUUGCACGGUAUCCAUCUG
4.
(SEQ ID NO: 4)
CUCCCACUGCUUCACUUGACUAGCC
5.
(SEQ ID NO: 5)
UUCUCACUACUGCACUUGACUAGUC
6.
(SEQ ID NO: 6)
UCCUUCAUUCCACCGGAGU
7.
(SEQ ID NO: 7)
UCCUUCAUUCCACCGGAGUCU
8.
(SEQ ID NO: 8)
CAGUGCAAUGUUAAAAGGGC
9.
(SEQ ID NO: 9)
CUAUCUGAGAGAUGGUGAUGACA
10.
(SEQ ID NO: 10)
UGUAAACAUCCCCGACUGGAAGCA
11.
(SEQ ID NO: 11)
UACAGUAGUCUGCACAUUGGUU
12.
(SEQ ID NO: 12)
UCUUGCGACCCGGGUUCGUUUCCCGGGCGGCGCACCA
13.
(SEQ ID NO: 13)
UGAUUAAGUCCCUGCCCUUUGUACACACCGCC
14.
(SEQ ID NO: 14)
CGGGCCGCCGGUGAAAUACCACUA
15.
(SEQ ID NO: 15)
UAGCACCAUCUGAAAUCGGU
16.
(SEQ ID NO: 16)
UCGAAACCGGGCAGAAGCACCA
17.
(SEQ ID NO: 17)
UGGAGUGUGACAAUGGUGUUUGA
18.
(SEQ ID NO: 18)
CAGUGCAAUGUUAAAAGGG
19.
(SEQ ID NO: 19)
UAGCAGCGGGAACAGUUCUG
20.
(SEQ ID NO: 20)
ACUGCAGUGAAGGCACUUGUA
21.
(SEQ ID NO: 21)
GGUCAAUGAUGUGUUGGCA
22.
(SEQ ID NO: 22)
UACUUCCAGGAACGGUGCACC
23.
(SEQ ID NO: 23)
ACAGUAGUCUGCACAUUGGUUA
24.
(SEQ ID NO: 24)
UGGCUCAGUUCAGCAGGAACAGUA
25.
(SEQ ID NO: 25)
UCCCCCAGGUGUGAUUCUGAUU
26.
(SEQ ID NO: 26)
AUUGGGGAGUGAGAGGGAGAGAACGCGGUCUGAGUGGU
27.
(SEQ ID NO: 27)
AAGAACUGCUAACUCAUGCCCCCAUGUA
28.
(SEQ ID NO: 28)
UGUAAACAUCCCCGACUGGAAG
29.
(SEQ ID NO: 29)
ACGCCCUUCCCCCCCUUCU
30.
(SEQ ID NO: 30)
UCGAUUCCCGGCCCAUGCACCA
31.
(SEQ ID NO: 31)
GAUAUAUACGCCAGUACCGCC
32.
(SEQ ID NO: 32)
GUCCAUAACAAAGCGGAGG
33.
(SEQ ID NO: 33)
GCCGGGUACUUUCGUAUUU
34.
(SEQ ID NO: 34)
AGAAGGUUGCGUGUUCAGGUCACGUCGGGGUCACCA
35.
(SEQ ID NO: 35)
UCUCCAAUGGAGGCGUGGGUUU
36.
(SEQ ID NO: 36)
CAUAAAGUAGAAAGCACUACUA
37.
(SEQ ID NO: 37)
GAGACCGCCUGGGAAUACCGGGUG
38.
(SEQ ID NO: 38)
UUUCGAUGGUAGUCGCCGUGCC
39.
(SEQ ID NO: 39)
CAGCCCUCGACACAAGGGUUUG
40.
(SEQ ID NO: 40)
UGCGCGUCCCCCGAAGAGG
41.
(SEQ ID NO: 41)
UGGGUUUACCUUGGGAGAACU
42.
(SEQ ID NO: 42)
CAUUCUGAAAGAACGUGUGGAAAACUAAUGACUGAGCA
43.
(SEQ ID NO: 43)
CACCCGGCUGUGUGCACAUGUGC
44.
(SEQ ID NO: 44)
GUUUCCGUAGUGUAGUGGUUAUCACGUUCGC
45.
(SEQ ID NO: 45)
AAGCUGCCAGUUGAAGAACUGU
46.
(SEQ ID NO: 46)
GGAGGUAGUAGGUUGUAUAGUU
47.
(SEQ ID NO: 47)
AAAAGCUGGGUUGAGAGGGCGAG
48.
(SEQ ID NO: 48)
GCCGCCGGUGAAAUACCACUACU
49.
(SEQ ID NO: 49)
UCGAAUCCGAGUCACGGCA
50.
(SEQ ID NO: 50)
CUUCUCACUACUGCACUUGACUAGUCUUA
51.
(SEQ ID NO: 51)
UGGAGUGUGACAAUGGUGUUU
52.
(SEQ ID NO: 52)
GAUUCCCGGCCAAUGCACC
53.
(SEQ ID NO: 53)
GGGAGAGAAAGGCAGUUCCUG
54.
(SEQ ID NO: 54)
UUUGGCAAUGGUAGAACUC
55.
(SEQ ID NO: 55)
CAGCAGCACACUGUGGUUUGUA
56.
(SEQ ID NO: 56)
AAAUAAGCUAUCGGGCCCAUACCCCG
57.
(SEQ ID NO: 57)
AUUGGUCGUGGUUGUAGUCCGUGCGAGAAUACCA
58.
(SEQ ID NO: 58)
CAAUCCUAAGCCAAAAGAACAA
59.
(SEQ ID NO: 59)
UGAGCAUGUAGACAAAGGUAACACUGAAG
60.
(SEQ ID NO: 60)
UGGCUCAGUUCAGCAGGAAC
61.
(SEQ ID NO: 61)
GUCCAAUAAAACAAAGCGGAGG
62.
(SEQ ID NO: 62)
UCCGCCCCCCGGCCCCGCGUCCUC
63.
(SEQ ID NO: 63)
UCUCACACGGAAAUCGCACCCGU
64.
(SEQ ID NO: 64)
UAAUGCCCCUAAAAAUCCUU
65.
(SEQ ID NO: 65)
UAAUCCUUGCUACCUGGGUGAGAG
66.
(SEQ ID NO: 66)
GUUCUACAGUCCGACAAU
67.
(SEQ ID NO: 67)
CGGUGCGCCGCGACCGGCUCCGGGACGGCUG
68.
(SEQ ID NO: 68)
UCUCACACAGAAAUCGCACCCGU
69.
(SEQ ID NO: 69)
UAGCAGCGGGAACAGUUCUGAA
70.
(SEQ ID NO: 70)
UGUAAACAUCCCCGACUGGAAGCU
71.
(SEQ ID NO: 71)
AGGGGCAGAGAGCGAGACUUU
72.
(SEQ ID NO: 72)
GUUCACUGAUGAGAGCAUUGUUCUGAGCCA
73.
(SEQ ID NO: 73)
UCAUUGGUCGUGGUUGUAGUCCGUGCGAGAAUACCA
74.
(SEQ ID NO: 74)
AUGCACCUGGGCAAGGAUUCUG
75.
(SEQ ID NO: 75)
AUCCCACUCCUGACACCUUU
76.
(SEQ ID NO: 76)
GGCCGCCGGUGAAAUACCACUACU
77.
(SEQ ID NO: 77)
GUCAGUUUGUCAAAUACCC
78.
(SEQ ID NO: 78)
UAGCACCAUUUGAAAUCG
79.
(SEQ ID NO: 79)
UUCAAAUCCCGGACGAGCCCA

1. A method of diagnosing lung cancer in a patient comprising the step of:
(i) determining the level of at least one RNA molecule in a blood sample from a patient, wherein the at least one RNA molecule comprises a nucleotide sequence according to SEQ ID NO: 1, is a fragment thereof, or has a sequence having at least 80% sequence identity thereto,
(ii) determining the levels of at least two RNA molecules in a blood sample from a patient, wherein the at least two RNA molecules comprise a first RNA molecule and a second RNA molecule,
wherein the first RNA molecule comprises a nucleotide sequence according to SEQ ID NO: 1, is a fragment thereof, or has a sequence having at least 80% sequence identity thereto and the second RNA molecule comprises a nucleotide sequence according to SEQ ID NO: 2, is a fragment thereof, or has a sequence having at least 80% sequence identity thereto, or
(iii) determining the levels of at least two RNA molecules in a blood sample from a patient, wherein the at least two RNA molecules comprise a first RNA molecule and a second RNA molecule,
wherein the first RNA molecule comprises a nucleotide sequence according to SEQ ID NO: 2, is a fragment thereof, or has a sequence having at least 80% sequence identity thereto and the second RNA molecule comprises a nucleotide sequence according to SEQ ID NO: 3, is a fragment thereof, or has a sequence having at least 80% sequence identity thereto.

2. The method of claim 1, wherein
with regard to (i), the level of at least one further RNA molecule selected from the group consisting of an RNA molecule comprising a nucleotide sequence according to SEQ ID NO: 2 to SEQ ID NO: 71, a fragment thereof, and a sequence having at least 80% sequence identity thereto is determined, or
wherein the level of at least one further RNA molecule selected from the group consisting of an RNA molecule comprising a nucleotide sequence according to SEQ ID NO: 2 to SEQ ID NO: 79, a fragment thereof, and a sequence having at least 80% sequence identity thereto is determined,
with regard to (ii), wherein the level of at least one further RNA molecule selected from the group consisting of an RNA molecule comprising a nucleotide sequence according to SEQ ID NO: 3 to SEQ ID NO: 71, a fragment thereof, and a sequence having at least 80% sequence identity thereto is determined, or
wherein the level of at least one further RNA molecule selected from the group consisting of an RNA molecule comprising a nucleotide sequence according to SEQ ID NO: 3 to SEQ ID NO: 79, a fragment thereof, and a sequence having at least 80% sequence identity thereto is determined, or
with regard to (iii), the level of at least one further RNA molecule selected from the group consisting of an RNA molecule comprising a nucleotide sequence according to SEQ ID NO: 1, SEQ ID NO: 4 to SEQ ID NO: 79, a fragment thereof, and a sequence having at least 80% sequence identity thereto is determined.

3-10. (canceled)

11. The method of claim 1,
wherein with regard to (i) the level of the at least one RNA molecule is compared to a reference level of said RNA molecule,
wherein the reference level is the level of the at least one RNA molecule determined empirically by measuring a number of reference blood samples from subjects known to be healthy (not suffering from lung cancer), and
wherein the level of the RNA molecule of SEQ ID NO: 1 above the reference level indicates that the patient suffers from lung cancer,
or
wherein with regard to (ii) or (iii), the levels of the at least two RNA molecules are compared to reference levels of said RNA molecules,
wherein the reference levels are the levels of the at least two RNA molecules determined empirically by measuring a number of reference blood samples from subjects known to be healthy (not suffering from lung cancer), and
wherein the level of the RNA molecules selected from the group consisting of SEQ ID NO: 1, SEQ ID NO: 2, SEQ ID NO: 3, above the reference level indicates that the patient suffers from lung cancer.

12. The method of claim 2,
wherein the levels of the at least two RNA molecules are compared to reference levels of said RNA molecules,
the reference levels are the levels of the at least two RNA molecules determined empirically by measuring a number of reference blood samples from subjects known to be healthy (not suffering from lung cancer), and
wherein the level of the RNA molecules selected from the group consisting of SEQ ID NO: 1, SEQ ID NO: 2, SEQ ID NO: 3, SEQ ID NO: 6, SEQ ID NO: 7, SEQ ID NO: 8, SEQ ID NO: 9, SEQ ID NO: 11, SEQ ID NO: 12, SEQ ID NO: 15, SEQ ID NO: 18, SEQ ID NO: 19, SEQ ID NO: 20, SEQ ID NO: 23, SEQ ID NO: 24, SEQ ID NO: 33, SEQ ID NO: 34, SEQ ID NO: 36, SEQ ID NO: 38, SEQ ID NO: 41, SEQ ID NO: 43, SEQ ID NO: 44, SEQ ID NO: 45, SEQ ID NO: 46, SEQ ID NO: 49, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 62, SEQ ID NO: 69, SEQ ID NO: 72, SEQ ID NO: 73, SEQ ID NO: 77, and/or SEQ ID NO: 78 above the reference level indicates that the patient suffers from lung cancer, and/or
the level of the RNA molecule selected from the group consisting of SEQ ID NO: 4, SEQ ID NO: 10, SEQ ID NO: 14, SEQ ID NO: 16, SEQ ID NO: 17, SEQ ID NO: 21, SEQ ID NO: 22, SEQ ID NO: 25, SEQ ID NO: 26, SEQ ID NO: 28, SEQ ID NO: 29, SEQ ID NO: 30, SEQ ID NO: 31, SEQ ID NO: 32, SEQ ID NO: 35, SEQ ID NO: 39, SEQ ID NO: 40, SEQ ID NO: 42, SEQ ID NO: 47, SEQ ID NO: 48, SEQ ID NO: 52, SEQ ID NO: 61, SEQ ID NO: 63, SEQ ID NO: 64, SEQ ID NO: 65, SEQ ID NO: 66, SEQ ID NO: 67, SEQ ID NO: 68, SEQ ID NO: 70, SEQ ID NO: 71, SEQ ID NO: 74, SEQ ID NO: 75, SEQ ID NO: 76, and/or SEQ ID NO: 79 below the reference level indicates that the patient suffers from lung cancer.

13-15. (canceled)

16. The method of claim 1,
wherein the level of the at least one RNA molecule is used in a mathematical computation to obtain a numerical value, or
the levels of the at least two RNA molecules are used in a mathematical computation to obtain a numerical value,
wherein the numerical value is obtained by summarizing the weighted levels of the at least two RNA molecules, and
wherein the numerical value is compared to an empirically determined cut-off score, and, wherein the cut-off score allows to classify a patient as suffering from lung cancer or as being healthy (not suffering from lung cancer).

17-19. (canceled)

20. A method of monitoring a patient suspected of having lung cancer or a patient suffering from lung cancer comprising the step of:
(i) determining the level of at least one RNA molecule in a blood sample from a patient, wherein the at least one RNA molecule comprises a nucleotide sequence according to SEQ ID NO: 1, is a fragment thereof, or has a sequence having at least 80% sequence identity thereto,
(ii) determining the levels of at least two RNA molecules in a blood sample from a patient, wherein the at least two RNA molecules comprise a first RNA molecule and a second RNA molecule,
wherein the first RNA molecule comprises a nucleotide sequence according to SEQ ID NO: 1, is a fragment thereof, or has a sequence having at least 80% sequence identity thereto and the second RNA molecule comprises a nucleotide sequence according to SEQ ID NO: 2, is a fragment thereof, or has a sequence having at least 80% sequence identity thereto, or
(iii) determining the levels of at least two RNA molecules in a blood sample from a patient, wherein the at least two RNA molecules comprise a first RNA molecule and a second RNA molecule,
wherein the first RNA molecule comprises a nucleotide sequence according to SEQ ID NO: 2, is a fragment thereof, or has a sequence having at least 80% sequence identity thereto and the second RNA molecule comprises a nucleotide sequence according to SEQ ID NO: 3, is a fragment thereof, or has a sequence having at least 80% sequence identity thereto.

21. The method of claim 20, wherein
with regard to (i), the level of at least one further RNA molecule selected from the group consisting of an RNA molecule comprising a nucleotide sequence according to SEQ ID NO: 2 to SEQ ID NO: 71, a fragment thereof, and a sequence having at least 80% sequence identity thereto is determined, or
wherein the level of at least one further RNA molecule selected from the group consisting of an RNA molecule comprising a nucleotide sequence according to SEQ ID NO: 2 to SEQ ID NO: 79, a fragment thereof, and a sequence having at least 80% sequence identity thereto is determined,
with regard to (ii), wherein the level of at least one further RNA molecule selected from the group consisting of an RNA molecule comprising a nucleotide sequence according to SEQ ID NO: 3 to SEQ ID NO: 71, a fragment thereof, and a sequence having at least 80% sequence identity thereto is determined, or
wherein the level of at least one further RNA molecule selected from the group consisting of an RNA molecule comprising a nucleotide sequence according to SEQ ID NO: 3 to SEQ ID NO: 79, a fragment thereof, and a sequence having at least 80% sequence identity thereto is determined, or
with regard to (iii), the level of at least one further RNA molecule selected from the group consisting of an RNA molecule comprising a nucleotide sequence according to SEQ ID NO: 1, SEQ ID NO: 4 to SEQ ID NO: 79, a fragment thereof, and a sequence having at least 80% sequence identity thereto is determined.

22-29. (canceled)

30. The method of claim 20,
wherein with regard to (i) the level of the at least one RNA molecule is compared to a reference level of said RNA molecule,
wherein the reference level is the level of the RNA molecule determined empirically by measuring a number of reference blood samples from subjects known to be healthy or subjects known to suffer from lung cancer, and
wherein said monitoring comprises determining the level of the at least one RNA molecule in a blood sample obtained from a patient at a first point in time and in at least one further blood sample obtained from the (same) patient at a later point in time and comparing said levels determined at the different time points
or,
wherein with regard to (ii) or (iii), the levels of the at least two RNA molecules are compared to reference levels of said RNA molecules,
wherein the reference levels are the levels of the at least two RNA molecules determined empirically by measuring a number of reference blood samples from subjects known to be healthy or known to suffer from lung cancer, and
wherein said monitoring comprises determining the levels of the at least two RNA molecules in a blood sample obtained from a patient at a first point in time and in at least one further blood sample obtained from the (same) patient at a later point in time and comparing said levels determined at the different time points.

31. The method of claim 21,
wherein the levels of the at least two RNA molecules are compared to reference levels of said RNA molecules,
wherein the reference levels are the levels of the at least two RNA molecules determined empirically by measuring a number of reference blood samples from subjects known to be healthy or known to suffer from lung cancer, and
wherein said monitoring comprises determining the levels of the at least two RNA molecules in a blood sample obtained from a patient at a first point in time and in at least one further blood sample obtained from the (same) patient at a later point in time and comparing said levels determined at the different time points.

32. (canceled)

33. The method of claim 1, wherein the lung cancer is small-cell lung carcinoma (SCLC) or non-small-cell lung carcinoma (NSCLC), preferably lung adenocarcinoma (LUAD) or lung squamous cell carcinoma (LUSC).

34. The method of claim 1, wherein the blood sample is whole blood or a blood fraction.

35. The method of claim 34, wherein the blood fraction is selected from the group consisting of a blood cell fraction, plasma, and serum.

36. The method of claim 35, wherein the blood cell fraction is a fraction of erythrocytes, leukocytes, and thrombocytes.

37. The method of claim 1, wherein the level is determined by sequencing, preferably next generation sequencing, nucleic acid hybridization, nucleic acid amplification, polymerase extension, mass spectroscopy or any combination thereof.

38-48. (canceled)

49. A kit for diagnosing lung cancer in a patient, or for monitoring a patient suspected of having lung cancer or a patient suffering from lung cancer, said kit comprises
(i) means for determining the level of at least one RNA molecule in a blood sample from a patient,
wherein the at least one RNA molecule comprises a nucleotide sequence according to SEQ ID NO: 1, is a fragment thereof, or has a sequence having at least 80% sequence identity thereto,
(ii) means for determining the levels of at least two RNA molecules in a blood sample from a patient, wherein the at least two RNA molecules comprise a first RNA molecule and a second RNA molecule,
wherein the first RNA molecule comprises a nucleotide sequence according to SEQ ID NO: 1, is a fragment thereof, or has a sequence having at least 80% sequence identity thereto and the second RNA molecule comprises a nucleotide sequence according to SEQ ID NO: 2, is a fragment thereof, or has a sequence having at least 80% sequence identity thereto, or
(iii) means for determining the levels of at least two RNA molecules in a blood sample from a patient, wherein the at least two RNA molecules comprise a first RNA molecule and a second RNA molecule,
wherein the first RNA molecule comprises a nucleotide sequence according to SEQ ID NO: 2, is a fragment thereof, or has a sequence having at least 80% sequence identity thereto and the second RNA molecule comprises a nucleotide sequence according to SEQ ID NO: 3, is a fragment thereof, or has a sequence having at least 80% sequence identity thereto.

50. The kit of claim 49,
wherein with regard to (i), the kit comprises means for determining the level of at least one further RNA molecule selected from the group consisting of an RNA molecule comprising a nucleotide sequence according to SEQ ID NO: 2 to SEQ ID NO: 71, a fragment thereof, and a sequence having at least 80% sequence identity thereto, or the kit comprises means for determining the level of at least one further RNA molecule selected from the group consisting of an RNA molecule comprising a nucleotide sequence according to SEQ ID NO: 2 to SEQ ID NO: 79, a fragment thereof, and a sequence having at least 80% sequence identity thereto,
wherein with regard to (ii), the kit comprises means for determining the level of at least one further RNA molecule selected from the group consisting of an RNA molecule comprising a nucleotide sequence according to SEQ ID NO: 3 to SEQ ID NO: 71, a fragment thereof, and a sequence having at least 80% sequence identity thereto, or the kit comprises means for determining the level of at least one further RNA molecule selected from the group consisting of an RNA molecule comprising a nucleotide sequence according to SEQ ID NO: 3 to SEQ ID NO: 79, a fragment thereof, and a sequence having at least 80% sequence identity thereto is used for diagnosing or monitoring lung cancer in the patient, or
wherein with regard to (iii), the kit comprises means for determining the level of at least one further RNA molecule selected from the group consisting of an RNA molecule comprising a nucleotide sequence according to SEQ ID NO: 1, SEQ ID NO: 4 to SEQ ID NO: 79, a fragment thereof, and a sequence having at least 80% sequence identity thereto.

51-60. (canceled)

61. The method of claim 20, wherein the blood sample is whole blood or a blood fraction.

62. The method of claim 61, wherein the blood fraction is selected from the group consisting of a blood cell fraction, plasma, and serum.

63. The method of claim 62, wherein the blood cell fraction is a fraction of erythrocytes, leukocytes, and thrombocytes.

64. The method of claim 20, wherein the level is determined by sequencing, preferably next generation sequencing, nucleic acid hybridization, nucleic acid amplification, polymerase extension, mass spectroscopy or any combination thereof.


SEQUENCE LISTING (79 sequences)

SEQ ID NO: 1 - RNA, 21 nt, Homo sapiens
  source 1..21: mol_type = other RNA
ttcacagtggctaagttctgc

SEQ ID NO: 2 - RNA, 22 nt, Homo sapiens
  source 1..22: mol_type = other RNA
gccgccggtgaaataccactac

SEQ ID NO: 3 - RNA, 19 nt, Homo sapiens
  source 1..19: mol_type = other RNA
attgcacggtatccatctg

SEQ ID NO: 4 - RNA, 25 nt, Homo sapiens
  source 1..25: mol_type = other RNA
ctcccactgcttcacttgactagcc

SEQ ID NO: 5 - RNA, 25 nt, Homo sapiens
  source 1..25: mol_type = other RNA
ttctcactactgcacttgactagtc

SEQ ID NO: 6 - RNA, 19 nt, Homo sapiens
  source 1..19: mol_type = other RNA
tccttcattccaccggagt

SEQ ID NO: 7 - RNA, 21 nt, Homo sapiens
  source 1..21: mol_type = other RNA
tccttcattccaccggagtct

SEQ ID NO: 8 - RNA, 20 nt, Homo sapiens
  source 1..20: mol_type = other RNA
cagtgcaatgttaaaagggc

SEQ ID NO: 9 - RNA, 23 nt, Homo sapiens
  source 1..23: mol_type = other RNA
ctatctgagagatggtgatgaca

SEQ ID NO: 10 - RNA, 24 nt, Homo sapiens
  source 1..24: mol_type = other RNA
tgtaaacatccccgactggaagca

SEQ ID NO: 11 - RNA, 22 nt, Homo sapiens
  source 1..22: mol_type = other RNA
tacagtagtctgcacattggtt

SEQ ID NO: 12 - RNA, 37 nt, Homo sapiens
  source 1..37: mol_type = other RNA
tcttgcgacccgggttcgtttcccgggcggcgcacca

SEQ ID NO: 13 - RNA, 32 nt, Homo sapiens
  source 1..32: mol_type = other RNA
tgattaagtccctgccctttgtacacaccgcc

SEQ ID NO: 14 - RNA, 24 nt, Homo sapiens
  source 1..24: mol_type = other RNA
cgggccgccggtgaaataccacta

SEQ ID NO: 15 - RNA, 20 nt, Homo sapiens
  source 1..20: mol_type = other RNA
tagcaccatctgaaatcggt

SEQ ID NO: 16 - RNA, 22 nt, Homo sapiens
  source 1..22: mol_type = other RNA
tcgaaaccgggcagaagcacca

SEQ ID NO: 17 - RNA, 23 nt, Homo sapiens
  source 1..23: mol_type = other RNA
tggagtgtgacaatggtgtttga

SEQ ID NO: 18 - RNA, 19 nt, Homo sapiens
  source 1..19: mol_type = other RNA
cagtgcaatgttaaaaggg

SEQ ID NO: 19 - RNA, 20 nt, Homo sapiens
  source 1..20: mol_type = other RNA
tagcagcgggaacagttctg

SEQ ID NO: 20 - RNA, 21 nt, Homo sapiens
  source 1..21: mol_type = other RNA
actgcagtgaaggcacttgta

SEQ ID NO: 21 - RNA, 19 nt, Homo sapiens
  source 1..19: mol_type = other RNA
ggtcaatgatgtgttggca

SEQ ID NO: 22 - RNA, 21 nt, Homo sapiens
  source 1..21: mol_type = other RNA
tacttccaggaacggtgcacc

SEQ ID NO: 23 - RNA, 22 nt, Homo sapiens
  source 1..22: mol_type = other RNA
acagtagtctgcacattggtta

SEQ ID NO: 24 - RNA, 24 nt, Homo sapiens
  source 1..24: mol_type = other RNA
tggctcagttcagcaggaacagta

SEQ ID NO: 25 - RNA, 22 nt, Homo sapiens
  source 1..22: mol_type = other RNA
tcccccaggtgtgattctgatt

SEQ ID NO: 26 - RNA, 38 nt, Homo sapiens
  source 1..38: mol_type = other RNA
attggggagtgagagggagagaacgcggtctgagtggt

SEQ ID NO: 27 - RNA, 28 nt, Homo sapiens
  source 1..28: mol_type = other RNA
aagaactgctaactcatgcccccatgta

SEQ ID NO: 28 - RNA, 22 nt, Homo sapiens
  source 1..22: mol_type = other RNA
tgtaaacatccccgactggaag

SEQ ID NO: 29 - RNA, 19 nt, Homo sapiens
  source 1..19: mol_type = other RNA
acgcccttcccccccttct

SEQ ID NO: 30 - RNA, 22 nt, Homo sapiens
  source 1..22: mol_type = other RNA
tcgattcccggcccatgcacca

SEQ ID NO: 31 - RNA, 21 nt, Homo sapiens
  source 1..21: mol_type = other RNA
gatatatacgccagtaccgcc

SEQ ID NO: 32 - RNA, 19 nt, Homo sapiens
  source 1..19: mol_type = other RNA
gtccataacaaagcggagg

SEQ ID NO: 33 - RNA, 19 nt, Homo sapiens
  source 1..19: mol_type = other RNA
gccgggtactttcgtattt

SEQ ID NO: 34 - RNA, 36 nt, Homo sapiens
  source 1..36: mol_type = other RNA
agaaggttgcgtgttcaggtcacgtcggggtcacca

SEQ ID NO: 35 - RNA, 22 nt, Homo sapiens
  source 1..22: mol_type = other RNA
tctccaatggaggcgtgggttt

SEQ ID NO: 36 - RNA, 22 nt, Homo sapiens
  source 1..22: mol_type = other RNA
cataaagtagaaagcactacta

SEQ ID NO: 37 - RNA, 24 nt, Homo sapiens
  source 1..24: mol_type = other RNA
gagaccgcctgggaataccgggtg

SEQ ID NO: 38 - RNA, 22 nt, Homo sapiens
  source 1..22: mol_type = other RNA
tttcgatggtagtcgccgtgcc

SEQ ID NO: 39 - RNA, 22 nt, Homo sapiens
  source 1..22: mol_type = other RNA
cagccctcgacacaagggtttg

SEQ ID NO: 40 - RNA, 19 nt, Homo sapiens
  source 1..19: mol_type = other RNA
tgcgcgtcccccgaagagg

SEQ ID NO: 41 - RNA, 21 nt, Homo sapiens
  source 1..21: mol_type = other RNA
tgggtttaccttgggagaact

SEQ ID NO: 42 - RNA, 38 nt, Homo sapiens
  source 1..38: mol_type = other RNA
cattctgaaagaacgtgtggaaaactaatgactgagca

SEQ ID NO: 43 - RNA, 23 nt, Homo sapiens
  source 1..23: mol_type = other RNA
cacccggctgtgtgcacatgtgc

SEQ ID NO: 44 - RNA, 31 nt, Homo sapiens
  source 1..31: mol_type = other RNA
gtttccgtagtgtagtggttatcacgttcgc

SEQ ID NO: 45 - RNA, 22 nt, Homo sapiens
  source 1..22: mol_type = other RNA
aagctgccagttgaagaactgt

SEQ ID NO: 46 - RNA, 22 nt, Homo sapiens
  source 1..22: mol_type = other RNA
ggaggtagtaggttgtatagtt

SEQ ID NO: 47 - RNA, 23 nt, Homo sapiens
  source 1..23: mol_type = other RNA
aaaagctgggttgagagggcgag

SEQ ID NO: 48 - RNA, 23 nt, Homo sapiens
  source 1..23: mol_type = other RNA
gccgccggtgaaataccactact

SEQ ID NO: 49 - RNA, 19 nt, Homo sapiens
  source 1..19: mol_type = other RNA
tcgaatccgagtcacggca

SEQ ID NO: 50 - RNA, 29 nt, Homo sapiens
  source 1..29: mol_type = other RNA
cttctcactactgcacttgactagtctta

SEQ ID NO: 51 - RNA, 21 nt, Homo sapiens
  source 1..21: mol_type = other RNA
tggagtgtgacaatggtgttt

SEQ ID NO: 52 - RNA, 19 nt, Homo sapiens
  source 1..19: mol_type = other RNA
gattcccggccaatgcacc

SEQ ID NO: 53 - RNA, 21 nt, Homo sapiens
  source 1..21: mol_type = other RNA
gggagagaaaggcagttcctg

SEQ ID NO: 54 - RNA, 19 nt, Homo sapiens
  source 1..19: mol_type = other RNA
tttggcaatggtagaactc

SEQ ID NO: 55 - RNA, 22 nt, Homo sapiens
  source 1..22: mol_type = other RNA
cagcagcacactgtggtttgta

SEQ ID NO: 56 - RNA, 26 nt, Homo sapiens
  source 1..26: mol_type = other RNA
aaataagctatcgggcccataccccg

SEQ ID NO: 57 - RNA, 34 nt, Homo sapiens
  source 1..34: mol_type = other RNA
attggtcgtggttgtagtccgtgcgagaatacca

SEQ ID NO: 58 - RNA, 22 nt, Homo sapiens
  source 1..22: mol_type = other RNA
caatcctaagccaaaagaacaa

SEQ ID NO: 59 - RNA, 29 nt, Homo sapiens
  source 1..29: mol_type = other RNA
tgagcatgtagacaaaggtaacactgaag

SEQ ID NO: 60 - RNA, 20 nt, Homo sapiens
  source 1..20: mol_type = other RNA
tggctcagttcagcaggaac

SEQ ID NO: 61 - RNA, 22 nt, Homo sapiens
  source 1..22: mol_type = other RNA
gtccaataaaacaaagcggagg

SEQ ID NO: 62 - RNA, 24 nt, Homo sapiens
  source 1..24: mol_type = other RNA
tccgccccccggccccgcgtcctc

SEQ ID NO: 63 - RNA, 23 nt, Homo sapiens
  source 1..23: mol_type = other RNA
tctcacacggaaatcgcacccgt

SEQ ID NO: 64 - RNA, 20 nt, Homo sapiens
  source 1..20: mol_type = other RNA
taatgcccctaaaaatcctt

SEQ ID NO: 65 - RNA, 24 nt, Homo sapiens
  source 1..24: mol_type = other RNA
taatccttgctacctgggtgagag

SEQ ID NO: 66 - RNA, 18 nt, Homo sapiens
  source 1..18: mol_type = other RNA
gttctacagtccgacaat

SEQ ID NO: 67 - RNA, 31 nt, Homo sapiens
  source 1..31: mol_type = other RNA
cggtgcgccgcgaccggctccgggacggctg

SEQ ID NO: 68 - RNA, 23 nt, Homo sapiens
  source 1..23: mol_type = other RNA
tctcacacagaaatcgcacccgt

SEQ ID NO: 69 - RNA, 22 nt, Homo sapiens
  source 1..22: mol_type = other RNA
tagcagcgggaacagttctgaa

SEQ ID NO: 70 - RNA, 24 nt, Homo sapiens
  source 1..24: mol_type = other RNA
tgtaaacatccccgactggaagct

SEQ ID NO: 71 - RNA, 21 nt, Homo sapiens
  source 1..21: mol_type = other RNA
aggggcagagagcgagacttt

SEQ ID NO: 72 - RNA, 30 nt, Homo sapiens
  source 1..30: mol_type = other RNA
gttcactgatgagagcattgttctgagcca

SEQ ID NO: 73 - RNA, 36 nt, Homo sapiens
  source 1..36: mol_type = other RNA
tcattggtcgtggttgtagtccgtgcgagaatacca

SEQ ID NO: 74 - RNA, 22 nt, Homo sapiens
  source 1..22: mol_type = other RNA
atgcacctgggcaaggattctg

SEQ ID NO: 75 - RNA, 20 nt, Homo sapiens
  source 1..20: mol_type = other RNA
atcccactcctgacaccttt

SEQ ID NO: 76 - RNA, 24 nt, Homo sapiens
  source 1..24: mol_type = other RNA
ggccgccggtgaaataccactact

SEQ ID NO: 77 - RNA, 19 nt, Homo sapiens
  source 1..19: mol_type = other RNA
gtcagtttgtcaaataccc

SEQ ID NO: 78 - RNA, 18 nt, Homo sapiens
  source 1..18: mol_type = other RNA
tagcaccatttgaaatcg

SEQ ID NO: 79 - RNA, 21 nt, Homo sapiens
  source 1..21: mol_type = other RNA
ttcaaatcccggacgagccca